SEQUENCE LISTING (31 sequences)

SEQ ID NO: 1 - protein, 112 aa, Artificial Sequence
  Synthetic polypeptide
ARVDQTPRSVTKETGESLTINCVLRDSSYALGSTYWYRKKSGSTNEESISKGGRYVETVNSGSKSFSLRINDLTVEDSGTYRCKYTSRLRREGPLSWDGNTVYGGGTVVTVN

SEQ ID NO: 2 - protein, 112 aa, Artificial Sequence
  Synthetic polypeptide
ARVDQTPRSVTKETGESLTINCVLRDTNYALGSTYWYRKKLGSTNEESISKGGRYVETVNSGSKSFSLRINDLTVEDSGTYRCKNLGHPMYIAGAICRPLPIYGGGTVVTVN

SEQ ID NO: 3 - protein, 112 aa, Artificial Sequence
  Synthetic polypeptide
ARVDQTPRSVTKETGESLTINCVLRDTSYALGSTYWYRKKSGSTNEESISKGGRYVETVNSGSKSFSLRINDLTVEDSGTYRCKFKSNTEFLNPLTVSMSTIYGGGTVVTVN

SEQ ID NO: 4 - protein, 112 aa, Artificial Sequence
  Synthetic polypeptide
ARVDQTPQTITKETGESLTINCVLRDSNYALGSTYWYRKKSGSTNEESISKGGRYVETVNSGSKSFSLRINDLTVEDSGTYRCKTQKAIPCNMNGYVCGVTIYGGGTVVTVN

SEQ ID NO: 5 - protein, 112 aa, Artificial Sequence
  Synthetic polypeptide
ARVDQTPQTITKETGESLTINCVLRDTNYALGSTYWYRKKLGSTNEESISKGGRYVETVNSGSKSFSLRINDLTVEDSGTYRCKNTSPWLTYSPWTVSGQTSYGGGTVVTVN

SEQ ID NO: 6 - protein, 112 aa, Artificial Sequence
  Synthetic polypeptide
ARVDQTPRSVTKETGESLTINCVLRDTSYALGSTYWYRKKLGSTNEESISKGGRYVETVNSGSKTFSLRINDLTVEDSGTYRCKVQTTWSYLSPYKIEFATVYGGGTVVTVN

SEQ ID NO: 7 - protein, 112 aa, Artificial Sequence
  Synthetic polypeptide
ARVDQTPRSVTKETGESLTINCVLRDTNYALGSTYWYRKKLGSTNEESISKGGRYVETVNSGSKSFSLRINDLTVEDSGTYRCKSTTPWNNLTSFTSERVTIYGGGTVVTVN

SEQ ID NO: 8 - protein, 115 aa, Artificial Sequence
  Synthetic polypeptide
ARVDQTPRSVTKETGESLTINCVLRDTSYALGSTYWYRKKLGSTNEESISKGGRYVETVNSGSKSFSLRINDLTVEDSGTYRCKMNQRYPCDNNSWWSLYCGTTVYGGGTVVTVN

SEQ ID NO: 9 - protein, 112 aa, Artificial Sequence
  Synthetic polypeptide
ARVDQTPQTITKETGESLTINCVLRDTSYALGSTYWYRKKSGSTNEESISKGGRYVETVNSGSKSFSLRINDLRVEDSGTYRCKYSSSWQKATEGVWEAMTEYGGGTVVTVN

SEQ ID NO: 10 - protein, 112 aa, Artificial Sequence
  Synthetic polypeptide
ARVDQTPRSVTKETGESLTINCVLRDTNYALGSTYWYRKKSGSTNEESISKGGRYVETVNSGSKSFSLRINDLTVEDSGTYRCKQSASLWIPNLLRWVPVISYGGGTVVTVN

SEQ ID NO: 11 - protein, 112 aa, Artificial Sequence
  Synthetic polypeptide
ARVDQTPRSVTKETGESLTINCVLRDTNYALGSTYWYRKKSGSTNEESISKGGRYVETVNSGSKSFSLRINDLTVEDSGTYRCKTSSKPLLVSHNVWSAWTEYGGGTVVTVN

SEQ ID NO: 12 - protein, 112 aa, Artificial Sequence
  Synthetic polypeptide
ARVDQTPRSVTKETGESLTINCVLRDTNYALGSTYWYRKKSGSTNEESISKGGRYVETVNSGSKSFSLRINDLTVEDSGTYRCKHQSSWRRQAPRVMEMQTLYGGGTVVTVN

SEQ ID NO: 13 - protein, 18 aa, Artificial Sequence
  Synthetic peptide
TTNSKREEKLFNVTSTLR

SEQ ID NO: 14 - protein, 9 aa, Artificial Sequence
  Synthetic peptide
  MISC_FEATURE (2)..(2): Xaa = Ser or Thr
  MISC_FEATURE (3)..(3): Xaa = Asn or Ser
DXXYALGST

SEQ ID NO: 15 - protein, 8 aa, Artificial Sequence
  Synthetic peptide
NEESISKG

SEQ ID NO: 16 - protein, 5 aa, Artificial Sequence
  Synthetic peptide
NSGSK

SEQ ID NO: 17 - DNA, 336 bp, Artificial Sequence
  Synthetic polynucleotide
gctcgagtggaccaaacaccgagatcagtaacaaaggagacgggcgaatcactgaccatcaactgtgtcctacgagatagtagctatgcattgggcagcacgtactggtatcgaaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaagtacacgtcgcgcctccggcgggaggggcccttgtcgtgggacgggaacacggtgtacggaggtggcactgtcgtgactgtgaat

SEQ ID NO: 18 - DNA, 336 bp, Artificial Sequence
  Synthetic polynucleotide
gctcgagtggaccaaacaccgagatcagtaacaaaggagacgggcgaatcactgaccatcaactgtgtcctacgagatactaactatgcattgggcagcacgtactggtatcgaaaaaaattgggctcaacaaacgaggagagcatatcgaaaggtggacgatatgttgaaacagttaacagcggatcaaagtccttttctttgagaattaatgatctaacagttgaagacagtggcacgtatcgatgcaagaacctcgggcacccgatgtacatcgcgggcgccatctgccggcccttgccgatctacggaggtggcactgtcgtgactgtgaat

SEQ ID NO: 19 - DNA, 336 bp, Artificial Sequence
  Synthetic polynucleotide
gctcgagtggaccaaacaccgagatcagtaacaaaggagacgggcgaatcactgaccatcaactgtgtcctacgagatactagctatgcattgggcagcacgtactggtatcgaaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaagttcaagagcaacaccgagttcctcaaccccttgacggtctcgatgagcacgatctacggaggtggcactgtcgtgactgtgaat

SEQ ID NO: 20 - DNA, 336 bp, Artificial Sequence
  Synthetic polynucleotide
gctcgagtggaccaaacaccgcaaacaataacaaaggagacgggcgaatcactgaccatcaactgtgtcctacgagatagtaactatgcattgggcagcacgtactggtatcgaaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaagacgcagaaggccatcccctgcaacatgaacgggtacgtgtgcggggtgacgatctacggaggtggcactgtcgtgactgtgaat

SEQ ID NO: 21 - DNA, 336 bp, Artificial Sequence
  Synthetic polynucleotide
gctcgagtggaccaaacaccgcaaacaataacaaaggagacgggcgaatcactgaccatcaactgtgtcctacgagatactaactatgcattgggcagcacgtactggtatcgaaaaaaattgggctcaacaaacgaggagagcatatcgaaaggtggacgatatgttgaaacagttaacagcggatcaaagtccttttctttgagaattaatgatctaacagttgaagacagtggcacgtatcgatgcaagaacacgtccccgtggctgacgtacagcccctggaccgtcagcgggcagacgtcgtacggaggtggcactgtcgtgactgtgaat

SEQ ID NO: 22 - DNA, 336 bp, Artificial Sequence
  Synthetic polynucleotide
gctcgagtggaccaaacaccgagatcagtaacaaaggagacgggcgaatcactgaccatcaactgtgtcctacgagatactagctatgcattgggcagcacgtactggtatcgaaaaaaattgggctcaacaaacgaggagagcatatcgaaaggtggacgatatgttgaaacagttaacagcggctcaaagaccttttctttgagaattaatgatctaacagttgaagacagtggcacgtatcgatgcaaggtgcagaccacctggtcctacttgagcccgtacaagatcgagttcgcgaccgtctacggaggtggcactgtcgtgactgtgaat

SEQ ID NO: 23 - DNA, 336 bp, Artificial Sequence
  Synthetic polynucleotide
gctcgagtggaccaaacaccgagatcagtaacaaaggagacgggcgaatcactgaccatcaactgtgtcctacgagatactaactatgcattgggcagcacgtactggtatcgaaaaaaattgggctcaacaaacgaggagagcatatcgaaaggtggacgatatgttgaaacagttaacagcggatcaaagtccttttctttgagaattaatgatctaacagttgaagacagtggcacgtatcgatgcaagtcgaccacgccctggaacaacctcacgtcgttcacgtccgagcgggtgaccatctacggaggtggcactgtcgtgactgtgaat

SEQ ID NO: 24 - DNA, 345 bp, Artificial Sequence
  Synthetic polynucleotide
gctcgagtggaccaaacaccgagatcagtaacaaaggagacgggcgaatcactgaccatcaactgtgtcctacgagatactagctatgcattgggcagcacgtactggtatcgaaaaaaattgggctcaacaaacgaggagagcatatcgaaaggtggacgatatgttgaaacagttaacagcggatcaaagtccttttctttgagaattaatgatctaacagttgaagacagtggcacgtatcgatgcaagatgaaccagcgctacccctgcgacaacaactcctggtggtccctgtactgcgggaccacggtctacggaggtggcactgtcgtgactgtgaat

SEQ ID NO: 25 - DNA, 336 bp, Artificial Sequence
  Synthetic polynucleotide
gctcgagtggaccaaacaccgcaaacaataacaaaggagacgggcgaatcactgaccatcaactgtgtcctacgagatactagctatgcattgggcagcacgtactggtatcgaaaaaaatcgggctcaacaaacgaggagagcatatcgaaaggtggacgatatgttgaaacagttaacagcggatcaaagtccttttctttgagaattaatgatctacgagttgaagacagtggcacgtatcgatgcaagtactcgagctcctggcagaaggccaccgagggcgtctgggaggcgatgacggagtacggaggtggcactgtcgtgactgtgaat

SEQ ID NO: 26 - DNA, 336 bp, Artificial Sequence
  Synthetic polynucleotide
gctcgagtggaccaaacaccgagatcagtaacaaaggagacgggcgaatcactgaccatcaactgtgtcctacgagatactaactatgcattgggcagcacgtactggtatcgaaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaagcagtcggcctcgctctggatcccgaacctcctccgctgggtgcccgtgatctcgtacggaggtggcactgtcgtgactgtgaat

SEQ ID NO: 27 - DNA, 336 bp, Artificial Sequence
  Synthetic polynucleotide
gctcgagtggaccaaacaccgagatcagtaacaaaggagacgggcgaatcactgaccatcaactgtgttctacgagatactaactatgcattgggcagcacgtactggtatcgaaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaagacgtcgtcgaagcccctcctcgtctcccacaacgtgtggtcggcctggacggagtacggaggtggcactgtcgtgactgtgaat

SEQ ID NO: 28 - DNA, 336 bp, Artificial Sequence
  Synthetic polynucleotide
gctcgagtggaccaaacaccgagatcagtaacaaaggagacgggcgaatcactgaccatcaactgtgtcctacgagatactaactatgcattgggcagcacgtactggtatcgaaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaagcaccagtcgtcctggaggcgccaggccccgcgcgtgatggagatgcagacgttgtacggaggtggcactgtcgtgactgtgaat

SEQ ID NO: 29 - protein, 238 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 30 - protein, 237 aa, Mus musculus
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNR

SEQ ID NO: 31 - protein, 236 aa, Canis familiaris
MRMFSVFTFMAYCHLLKAFTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASER

(19) United States
(12) Patent Application Publication
(10) Pub. No.: US 2024/0270851 A1
(43) Pub. Date: Aug. 15, 2024

(54) CROSS SPECIES SINGLE DOMAIN ANTIBODIES TARGETING PD-L1 FOR TREATING SOLID TUMORS

(71) Applicant: THE U.S.A., as represented by the Secretary, Department of Health and Human Services, Bethesda, MD (US)

(72) Inventors: Mitchell Ho, Urbana, MD (US); Glenn Merlino, Vienna, VA (US); Dan Li, Rockville, MD (US); Hejiao English, Columbia, MD (US); Chi-Ping Day, Rockville, MD (US)

(73) Assignee: The U.S.A., as represented by the Secretary, Department of Health and Human Services, Bethesda, MD (US)

(21) Appl. No.: 18/567,990

(22) PCT Filed: Jun. 6, 2022
(86) PCT No.: PCT/US2022/032378

§ 371 Date: Dec. 7, 2023

Related U.S. Application Data
(60) Provisional application No. 63/208,755, filed on Jun. 9, 2021.

(51) Int. Cl. C07K 16/28; A61K 39/00; A61P 35/00; C12N 15/63

(52) U.S. Cl. CPC C07K 16/2827; A61K 39/4631; A61K 39/464402; A61P 35/00; C12N 15/63; C07K 2317/31; C07K 2317/52; C07K 2317/565; C07K 2317/569

(57) ABSTRACT

Single-domain shark variable new antigen receptor (V_{NAR}) monoclonal antibodies that specifically bind programmed death-ligand 1 (PD-L1) are described. The PD-L1-specific V_{NAR} antibodies are capable of binding PD-L1-expressing tumor cells from human, mouse and canine origin. Immune cells expressing chimeric antigen receptors (CARs) developed using the V_{NAR} antibodies can be used to kill PD-L1-positive tumor cells, for example in animal models of liver cancer and breast cancer.

### CROSS REFERENCE TO RELATED APPLICATIONS

This application claims the benefit of U.S. Provisional Application No. 63/208,755, filed Jun. 9, 2021, which is herein incorporated by reference in its entirety.

### ACKNOWLEDGMENT OF GOVERNMENT SUPPORT

This invention was made with government support under project numbers Z01 BC010891 and ZIA BC008756 awarded by the National Cancer Institute, National Institutes of Health. The government has certain rights in the invention.

### FIELD

This disclosure concerns shark variable new antigen receptor (V_{NAR}) single-domain antibodies that specifically bind both mouse and human programmed death-ligand 1 (PD-L1) and their use in cancer immunotherapy and detection of PD-L1 positive tumors.

### BACKGROUND

Adoptive cell therapy (ACT), particularly using T cells genetically engineered with chimeric antigen receptors (CAR T cells), has shown great potency as one of the most effective cancer immunotherapies (Rosenberg et al., Nat Rev Cancer 2008; 8(4):299-308; Rosenberg and Restifo, Science 2015; 348(6230):62-68; Kochenderfer et al., Blood 2010; 116(20):4099-4102). CARs are synthetic receptors consisting of an extracellular domain, a hinge region, a transmembrane domain, and intracellular signaling domains (such as CD3ζ, CD28, 41BB) that initiate T cell activation (Maher et al., Nat Biotechnol 2002; 20(1):70-75; Imai et al., Leukemia 2004; 18(4):676-684; Song et al., Cancer Res 2011; 71(13):4617-4627). CARs can promote non-major histocompatibility complex (MHC)-restricted recognition of cell surface components, bind tumor antigens directly, and trigger a dramatic T cell anti-tumor response (Gross et al., Proc Natl Acad Sci USA 1989; 86(24):10024-10028). CAR T cells targeting B cell antigen CD19 have shown breakthrough clinical success in patients with advanced B cell lymphoma, which led to their approval by the U.S. Food and Drug Administration (FDA) (Kochenderfer et al., Blood 2010; 116(20):4099-4102; Kochenderfer and Rosenberg, Nat Rev Clin Oncol 2013; 10(5):267-276). However, the translation of CAR T cells to solid tumors is more challenging because of a lack of appropriate antigenic targets and the complex immunosuppressive tumor microenvironment (TME) (European Association For The Study Of The Liver et al., J Hepatol 2012; 56(4):908-943).

Glypican-2 (GPC2) (Li et al., Proc Natl Acad Sci USA 2017; 114(32):E6623-E6631), GPC3 (Li et al., Gastroenterology 2020; 158(8):2250-2265), and mesothelin (Lv et al., J Hematol Oncol 2019; 12:18; Zhang et al., Cell Death Dis 2019; 10(7):476; Hassan et al., Mol Cancer Ther 2022, doi:10.1158/1535-7163.MCT-22-0073; Liu et al., Proc Natl Acad Sci USA 119(19):e2202439119, 2022) are potential antigens for CAR T therapy in the treatment of solid tumors. However, not all tumors express highly specific surface antigens that are suitable for CAR recognition. Programmed death-ligand 1 (PD-L1 or CD274) is aberrantly expressed on multiple tumor types through oncogenic signaling (Sun et al., Immunity 2018; 48(3):434-452) and induction by pro-inflammatory factors such as IFN-γ in the immune-reactive TME (Dong et al., Nat Med 2002; 8(8):793-800). PD-L1 expressed on tumors can induce T-cell tolerance and avoid immune destruction through binding with its ligand PD-1 on T cells, which may inhibit the effect of CAR T cells in solid tumors (Weinstock and McDermott, Ther Adv Urol 2015; 7(6):365-377). Clinically, antibody-based PD-1/PD-L1 antagonists induce durable tumor inhibition, especially in melanoma, non-small cell lung cancer, and renal cancer. However, the response rate is poor in other types of advanced solid tumor (Sznol, Cancer J 2014; 20(4):290-295). PD-L1-targeted camelid V_{H}H-nanobody-based CAR T cells were shown to delay tumor growth in a syngeneic mouse melanoma model (Xie et al., Proc Natl Acad Sci USA 2019; 116(16):7624-7631). Moreover, PD-L1-targeted CAR natural killer (NK) cells inhibited the growth of triple negative breast cancer (TNBC), lung cancer, and bladder tumors engrafted in NOD scid gamma (NSG) mice (Fabian et al., J Immunother Cancer 2020; 8(1):e000450). Furthermore, bi-specific Trop2/PD-L1 CAR-T cells targeting both Trop2 and PD-L1 demonstrated improved killing effect of CAR-T cells in gastric cancer (Zhao et al., Am J Cancer Res 2019; 9(8):1846-1856).

### SUMMARY

Described herein are single-domain shark variable new antigen receptor (V_{NAR}) monoclonal antibodies that specifically bind PD-L1. The disclosed V_{NAR} antibodies are capable of binding PD-L1-expressing tumor cells from human, mouse and in some instances, canine origin. Immune cells expressing CARs based on the disclosed V_{NAR} antibodies can be used to kill PD-L1-positive tumor cells, for example in a subject with a PD-L1-positive cancer, such as in a subject with liver cancer or breast cancer. The present disclosure provides the first report of human and mouse cross-reactive PD-L1 antibodies and the first disclosure of single-domain PD-L1 antibodies.

Provided herein are polypeptides (for example, single-domain monoclonal antibodies) that bind, such as specifically bind, PD-L1. In some examples, the polypeptides (for example, single-domain monoclonal antibodies) bind to more than one species of PD-L1, such as human and mouse PD-L1, or human, mouse, and canine PD-L1. In some embodiments, the polypeptide includes one or more complementarity determining region (CDR) sequences, and/or one or both hypervariable (HV) regions, of antibody B2, F5, A11, A3, A9, A2, A10, A7, A6, C4, A1 or D12 provided herein. Also provided herein are conjugates that include a disclosed polypeptide. In some examples, provided are fusion proteins (such as Fc fusion proteins), chimeric antigen receptors (CARs), CAR-expressing immune cells (such as T cells, natural killer cells and macrophages), immunoconjugates (such as immunotoxins), multi-specific antibodies (such as bispecific antibodies), antibody-drug conjugates (ADCs), antibody-nanoparticle conjugates, and antibody-radioisotope conjugates (such as for immunoPET imaging) that include a polypeptide (for example, a single-domain monoclonal antibody) disclosed herein.

Also provided herein are nucleic acid molecules and vectors encoding the PD-L1-specific polypeptides (for example, antibodies), fusion proteins, CARs, immunoconjugates (such as immunotoxins), and multi-specific antibodies disclosed herein. Isolated cells that include a nucleic acid or vector encoding a PD-L1-specific polypeptide or CAR are further provided.

Compositions that include a pharmaceutically acceptable carrier and a PD-L1-specific polypeptide, fusion protein, CAR, immunoconjugate, ADC, multi-specific antibody, antibody-nanoparticle conjugate, isolated nucleic acid molecule or vector disclosed herein are also provided by the present disclosure. Also provided are solid supports, such as beads (e.g., glass, magnetic, or plastic beads), multiwell plates, paper, or nitrocellulose that include one or more PD-L1-specific polypeptides (such as single-domain monoclonal antibodies) provided herein.

Methods of detecting PD-L1 in a sample, and methods of diagnosing a subject as having a PD-L1-positive cancer, are further provided. In some embodiments, the methods include contacting a sample obtained from the subject with a polypeptide (for example, a single-domain monoclonal antibody) disclosed herein, and detecting binding of the polypeptide to the sample.

Also provided is a method of treating a PD-L1-positive cancer in a subject. In some embodiments, the method includes administering to the subject a therapeutically effective amount of a polypeptide (for example, a single-domain monoclonal antibody) disclosed herein, or administering to the subject a therapeutically effective amount of a fusion protein, CAR (or CAR-expressing immune cells), immunoconjugate (such as an immunotoxin), ADC, multi-specific antibody, or antibody-nanoparticle conjugate comprising a polypeptide disclosed herein, or a nucleic acid molecule or vector encoding a disclosed polypeptide. In some examples, such a method is used in combination with one or more other anti-cancer therapies, such as administration of a therapeutically effective amount of one or more anti-PD-1 monoclonal antibodies (mAbs).

The foregoing and other objects and features of the disclosure will become more apparent from the following detailed description, which proceeds with reference to the accompanying figures.

### BRIEF DESCRIPTION OF THE DRAWINGS

FIGS. 1A-1H: Isolation of anti-PD-L1 single domain antibodies by phage display from an engineered shark V_{NAR} phage library. (FIG. 1A) Schematic of library construction. The variable regions are shown as CDR1, HV2, HV4, and CDR3. The two canonical cysteines (21C and 82C) are in white circles, while the non-canonical cysteines are in black circles. The C29Y mutation is labeled. Disulfide bridges are represented by solid black lines, whereas the dotted line represents elimination of a disulfide bridge. A pair of primers was used to amplify the randomized CDR3 region. V_{NAR} fragments were assembled with vector backbone and the assembled vectors were electroporated into TG1 cells to generate the library. (FIG. 1B) Table comparing the semi-synthetic 18AA CDR3 shark V_{NAR} library with pre-synthetic shark V_{NAR} library. (FIG. 1C) Pie charts showing the percentage of average nucleotide (ACTG) ratio at each randomization NNS. (FIG. 1D) Phage-displayed single-domain antibody clones were identified against recombinant mouse PD-L1-His after four rounds of panning. A gradual increase in phage titers was observed during each round of panning. (FIG. 1E) Polyclonal phage ELISA from the output phage of each round of panning. (FIGS. 1F-1H) Monoclonal phage ELISA analysis of cross-reactivity of PD-L1 binders B2 (FIG. 1F), A11 (FIG. 1G), and F5 (FIG. 1H) to mouse PD-L1 and human PD-L1 protein within His-tag or hFc-tag formats.

FIGS. 2A-2E: Verification of specific binding and blocking ability of anti-PD-L1 shark V_{NAR}s. (FIG. 2A) Schematic design for constructing PD-L1 KO MDA-MB-231 cell line using CRISPR-Cas9 method. Two sgRNAs were designed to target the promoter of the endogenous PD-L1 gene. Single PD-L1 KO clones were validated by Western blot and flow cytometry. (FIG. 2B) The cross-reactive binding of anti-PD-L1 V_{NAR}s to native PD-L1 as determined by flow cytometry. Three different tumor cell lines (human breast cancer cell line MDA-MB-231, murine melanoma cell line B8979HC, and canine tumor cell line Jones) and PD-L1 knockout (KO) MDA-MB-231 were stained with V_{NAR}s. (FIG. 2C) Epitope mapping of individual B2, F5, and A11. Sequence alignment of PD-L1 extracellular domain (ECD) region of human, murine, and canine (SEQ ID NOs: 29, 30 and 31 respectively). The conserved residues are marked with asterisks (*), the residues with similar properties between variants are marked with colons (:) and the residues with marginally similar properties are marked with periods (.). The main binding residues of the hPD-L1 identified previously that interact with PD-1 are shaded (residues 54, 56, 115, 121, 123 and 134 of human PD-L1 of SEQ ID NO: 29). The binding peptides of B2 to hPD-L1 are highlighted (residues 181-198 of SEQ ID NO: 29). (FIG. 2D) Binding kinetics of B2-hFc to hPD-L1 protein. (FIG. 2E) Blocking activity of V_{NAR}-hFc to the interaction of hPD-L1 and hPD-1 as determined by the Octet platform.

FIGS. 3A-3G: PD-L1 specific V_{NAR}-based CAR T cells exhibit antigen specific cytotoxicity on MDA-MB-231. (FIG. 3A) Surface PD-L1 expression on multiple tumor types as determined by flow cytometry. (FIG. 3B) Lentiviral construct of PD-L1 specific V_{NAR}-based CAR T cell where CAR and hEGFRt are expressed separately by the self-cleaving T2A ribosomal skipping sequence. (FIG. 3C) The expression of hEGFRt on T cells indicates the transduction efficiency of PD-L1-targeted CAR T cells detected by flow cytometry. Mock control cells are untransduced T cells. (FIG. 3D) Cytolytic activity of PD-L1-targeted CAR T cells after 24 hours of incubation with MDA-MB-231 in a 2-fold dose dependent manner at high effector:target (E:T) ratio (maximum 100:1) or low E:T ratio (minimum 0.3125:1) for 24 hours and 96 hours. (FIG. 3E) Supernatants were collected from the low E:T ratio panels (5:1 and 2.5:1) in FIG. 3C, and TNF-α, IL-2, and IFN-γ production were measured by ELISA. (FIG. 3F) Specific killing of CAR (B2) T cells on WT MDA-MB-231 and PD-L1 KO MDA-MB-231 cells after 24 hours of co-culture. (FIG. 3G) Varying concentrations of soluble B2 nanobody were included in the B2 CAR-tumor cell incubation setup at E:T ratio of 1:1. The killing by CAR (B2) T cells was observed for 24 hours and 48 hours after incubation using a luciferase cytolytic assay. Tumor cells alone and with mock T cells in the presence of B2 nanobody were used as controls. Statistical analyses are shown from three independent experiments. Values represent mean±SEM. **P<0.01, ***P<0.001, ns, not significant.

FIGS. 4A-4F: CAR (B2) T cells lysed Hep3B tumors by targeting inducible PD-L1. (FIG. 4A) Inducible PD-L1 expression in Hep3B cells upon 50 μg/ml IFN-γ stimulation followed by depletion of IFN-γ at 24 hours. (FIG. 4B) Inducible PD-L1 expression in Hep3B cells after 24 hours incubation with CAR (B2) T at an E/T ratio of 1:2. IFN-γ level in the cell supernatants of CAR (CD19) T or CAR (B2) T cells co-cultured with Hep3B cell. (FIG. 4C) Cytolytic activity of CAR (B2) T cells on Hep3B tumor cells after 24 hours and 96 hours of incubation at various E:T ratios. (FIG. 4D) Schematic of the Hep3B xenograft NSG model i.p. infused with 5 million CAR (B2) T cells and CAR (CD19) T cells 12 days after tumor inoculation. (FIG. 4E) Representative bioluminescence image of Hep3B tumor growth in the xenograft model shown in FIG. 4D. (FIG. 4F) Tumor bioluminescence growth curve of mice treated in FIG. 4E.

FIGS. 5A-5G: Application of bispecific anti-GPC3 and anti-PD-L1 CAR T cells (Bi-hYP7−B2) in HCC therapy in vitro. (FIG. 5A) Incubation of Hep3B tumor cells with GPC3-targeted CAR (hYP7) and untransduced T cells (mock) for 24 hours at various E:T ratios. The cytolytic activity of CAR (hYP7) T cells was measured by tumor cells expressing luciferase. (FIG. 5B) IFN-γ secretion in the supernatants was measured by ELISA. (FIG. 5C) Surface PD-L1 expression on the Hep3B tumor cells was detected after 24 hours incubation with CAR (CD19) T and CAR (hYP7) T cells at various E:T ratios using flow cytometry. (FIG. 5D) Schematic design of bispecific hYP7−B2 CAR T cells. The activated T cells were co-transduced with CAR (hYP7) and CAR (B2) lentivirus to co-express both hYP7 scFv and B2 V_{NAR} on the CAR T cells as the recognition domain. (FIG. 5E) Table of experimental groups of bispecific CAR (hYP7−B2) T cells and combination CAR (hYP7) T cells with CAR (B2) T cells. (FIG. 5F) Cytolytic activity of bispecific CAR (hYP7−B2) T cells on Hep3B cells after 24 hours and 96 hours of incubation in vitro. (FIG. 5G) TNF-α, IL-2, and IFN-γ production in the co-culture supernatant from FIG. 5C were measured by ELISA.

FIGS. 6A-6E: Combined CAR (B2) with CAR (hYP7) T cells achieve a synergistic anti-tumor effect in vivo. (FIG. 6A) Experimental schematic of the in vivo study. A peritoneal Hep3B mouse model was established via i.p. injection of Hep3B GL (Day −12) followed by i.v. infusion of 5 million CAR (hYP7) T cells, CAR (CD19) T cells, CAR (B2) T cells, Bi-hYP7−B2 CAR T cells, or a combination of 2.5 million CAR (hYP7) T cells and 2.5 million CAR (B2) T cells (referred to as “hYP7 CAR+B2 CAR”) at Day 0. (FIG. 6B) In comparison with CAR (CD19) T cells, both CAR (hYP7) T and CAR (B2) T cells individually inhibited tumor growth in xenografts. Bi-hYP7−B2 CAR T cells failed to regress tumor burden and treatment with the bispecific CAR was less effective than mono-specific CAR-T cells, whereas the combination group hYP7 CAR+B2 CAR showed a significant synergistic anti-tumor effect in xenografts. (FIG. 6C) Mice receiving CAR (B2) T, hYP7 CAR+B2 CAR T, or Bi-hYP7−B2 CAR T cells had much higher absolute CD3+CAR+ T cell counts in blood compared with those receiving CAR (CD19) T or CAR (hYP7) T cells on week 2 after infusion (left to right: hYP7 CAR, B2 CAR, hYP7 CAR+B2 CAR, Bi-hYP7−B2 CAR and CD19 CAR). (FIG. 6D) In both CD4+ and CD8+ T subpopulations, CAR (hYP7) T showed higher proportion of memory stem cell-like (Tscm) T cells in mice than other CAR T cells, whereas B2-related CAR T cells had a higher proportion of effector memory (Tem) T cells than CAR (hYP7) T. (FIG. 6E) In vivo, CAR (hYP7) T cells expressed lower levels of PD-1 and LAG-3 than B2-related CAR T cells on week 2 after infusion (left to right: hYP7 CAR, B2 CAR, hYP7 CAR+B2 CAR, Bi-hYP7−B2 CAR and CD19 CAR).

FIGS. 7A-7H: Tumor regression in the orthotopic MDA-MB-231 xenograft mouse model by CAR (B2) T cell infusion. (FIG. 7A) Schematic of the MDA-MB-231 orthotopic xenograft NSG model i.v. infused with 5 million CAR (B2) T cells and CAR (CD19) CAR T cells after 17 days of tumor inoculation. (FIG. 7B) Representative bioluminescence images of MDA-MB-231 tumor growth in the orthotopic model shown in FIG. 7A. (FIG. 7C) Tumor size of MDA-MB-231 in the orthotopic model treated in FIG. 7A measured by a digital caliper. Values represent each single mouse. ***P<0.001. (FIG. 7D) Body weight of mice shown in FIG. 7A. Values shown represent mean±SEM. (FIG. 7E) Representative pictures showing the restriction of tumor metastasis in CAR (B2) T cell infusion mouse. (FIG. 6F) CAR (B2) T cell persistence and (FIG. 7G) ex vivo killing on MDA-MB-231 tumor cells after 3 weeks of CAR T cell infusion. (FIG. 7H) Detection of PD-L1 expression in MDA-MB-231 tumor xenograft by Western blotting.

FIG. 8: Flow cytometry analysis of PD-L1 expression and T cell exhaustion markers (PD-1, LAG-3, and TIM-3). Shown is expression by mock T cells and anti-PD-L1 shark V_{NAR}-based CAR T cells (B2, F5, and A11).

FIG. 9: Clustal Omega sequence alignment of V_{NAR} antibodies B2 (SEQ ID NO: 1), F5 (SEQ ID NO: 2), A11 (SEQ ID NO: 3), A3 (SEQ ID NO: 4), A9 (SEQ ID NO: 5), A2 (SEQ ID NO: 6), A10 (SEQ ID NO: 7), A7 (SEQ ID NO: 8), A6 (SEQ ID NO: 9), C4 (SEQ ID NO: 10), A1 (SEQ ID NO: 11) and D12 (SEQ ID NO: 12). The CDR1, CDR3, HV2 and HV4 of each antibody, according to the V_{NAR} annotation described in Stanfield et al. (Science 305:1770-1773, 2004) and Fennell et al. (J Mol Biol 400:155-170, 2010), are underlined.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on May 23, 2022, 26.8 KB, which is incorporated by reference herein. In the accompanying sequence listing:
SEQ ID NO: 1 is the amino acid sequence of V_{NAR} B2.
SEQ ID NO: 2 is the amino acid sequence of V_{NAR} F5.
SEQ ID NO: 3 is the amino acid sequence of V_{NAR} A11.
SEQ ID NO: 4 is the amino acid sequence of V_{NAR} A3.
SEQ ID NO: 5 is the amino acid sequence of V_{NAR} A9.
SEQ ID NO: 6 is the amino acid sequence of V_{NAR} A2.
SEQ ID NO: 7 is the amino acid sequence of V_{NAR} A10.
SEQ ID NO: 8 is the amino acid sequence of V_{NAR} A7.
SEQ ID NO: 9 is the amino acid sequence of V_{NAR} A6.
SEQ ID NO: 10 is the amino acid sequence of V_{NAR} C4.
SEQ ID NO: 11 is the amino acid sequence of V_{NAR} A1.
SEQ ID NO: 12 is the amino acid sequence of V_{NAR} D12.
SEQ ID NO: 13 is the amino acid sequence of a peptide from human PD-L1.
SEQ ID NO: 14 is a consensus V_{NAR} CDR1 amino acid sequence.
SEQ ID NO: 15 is a consensus V_{NAR} HV2 amino acid sequence.
SEQ ID NO: 16 is a consensus V_{NAR} HV4 amino acid sequence.
SEQ ID NO: 17 is a nucleotide sequence encoding V_{NAR} B2.
SEQ ID NO: 18 is a nucleotide sequence encoding V_{NAR} F5.
SEQ ID NO: 19 is a nucleotide sequence encoding V_{NAR} A11.
SEQ ID NO: 20 is a nucleotide sequence encoding V_{NAR} A3.
SEQ ID NO: 21 is a nucleotide sequence encoding V_{NAR} A9.
SEQ ID NO: 22 is a nucleotide sequence encoding V_{NAR} A2.
SEQ ID NO: 23 is a nucleotide sequence encoding V_{NAR} A10.
SEQ ID NO: 24 is a nucleotide sequence encoding V_{NAR} A7.
SEQ ID NO: 25 is a nucleotide sequence encoding V_{NAR} A6.
SEQ ID NO: 26 is a nucleotide sequence encoding V_{NAR} C4.
SEQ ID NO: 27 is a nucleotide sequence encoding V_{NAR} A1.
SEQ ID NO: 28 is a nucleotide sequence encoding V_{NAR} D12.

```
SEQ ID NO: 29 is the amino acid sequence of human PD-L1 ECD.
    1 MRIFAVFIFM TYWHLLNAFT VTVPKDLYVV EYGSNMTIEC KFPVEKQLDL AALIVYWEME
  61 DKNIIQFVHG EEDLKVQHSS YRQRARLLKD QLSLGNAALQ ITDVKLQDAG VYRCMISYGG
121 ADYKRITVKV NAPYNKINQR ILVVDPVTSE HELTCQAEGY PKAEVIWTSS DHQVLSGKTT
181 TINSKREEKL FNVTSTLRIN TTTNEIFYCT FRRLDPEENH TAELVIPELP LAHPPNER
SEQ ID NO: 30 is the amino acid sequence of mouse PD-L1 ECD.
    1 MRIFAGIIFT ACCHLLRAFT ITAPKDLYVV EYGSNVTMEC RFPVERELDL LALVVYWEKE
  61 DEQVIQFVAG EEDLKPQHSN FRGRASLPKD QLLKGNAALQ ITDVKLQDAG VYCCIISYGG
121 ADYKRITLKV NAPYRKINQR ISVDPATSEH ELICQAEGYP EAEVIWINSD HQPVSGKRSV
181 TTSRTEGMLL NVTSSLRVNA TANDVFYCTF WRSQPGQNHT AELIIPELPA THPPQNR
SEQ ID NO: 31 is the amino acid sequence of canine PD-L1 ECD.
    1 MRMFSVFTFM AYCHLLKAFT ITVSKDLYVV EYGGNVTMEC KFPVEKQLNL FALIVYWEME
  61 DKKIIQFVNG KEDLKVQHSS YSQRAQLLKD QLFLGKAALQ ITDVRLQDAG VYCCLIGYGG
121 ADYKRITLKV HAPYRNISQR ISVDPVTSEH ELMCQAEGYP EAEVIWTSSD HRVLSGKTTI
181 TNSNREEKLF NVTSTLNINA TANEIFYCTF QRSGPEENNT AELVIPERLP VPASER
```

### DETAILED DESCRIPTION

A major challenge in the development of CAR T cells for solid tumors is the lack of targetable antigens. Checkpoint molecule PD-L1 is highly expressed on many tumors in a constitutive or interferon-gamma (IFNγ)-inducible manner. IFN-γ is the key functional cytokine released from effector T cells; however, the increased expression of PD-L1 on tumor cells binding to PD-1 on effector T cells results in T cell exhaustion, and inhibition of T cell functions (Chen and Han, J Clin Invest 2015; 125(9):3384-3391). In the studies disclosed herein, it was hypothesized that the development of CAR T cells targeting PD-L1 would kill solid tumors via recognizing constitutive or inducible expression of PD-L1 in the tumor immunosuppressive microenvironment. To test this hypothesis, a panel of anti-PD-L1 nanobodies was isolated from a newly established semi-synthetic nurse shark V_{NAR} library. The B2 clone showed specific binding ability to naïve PD-L1 and cross-reacted with both human and mouse antigens. B2 also functionally blocked the interaction of PD-L1 to PD-1. Moreover, nanobody-based CAR T cells showed much higher transduction efficiency than scFv-based CAR T cells.

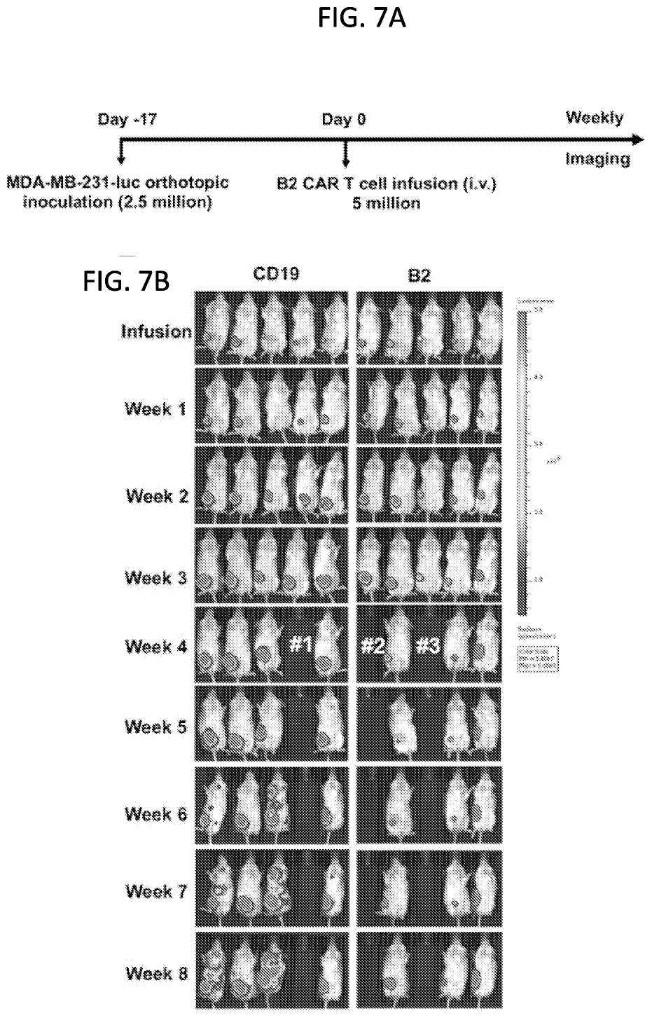

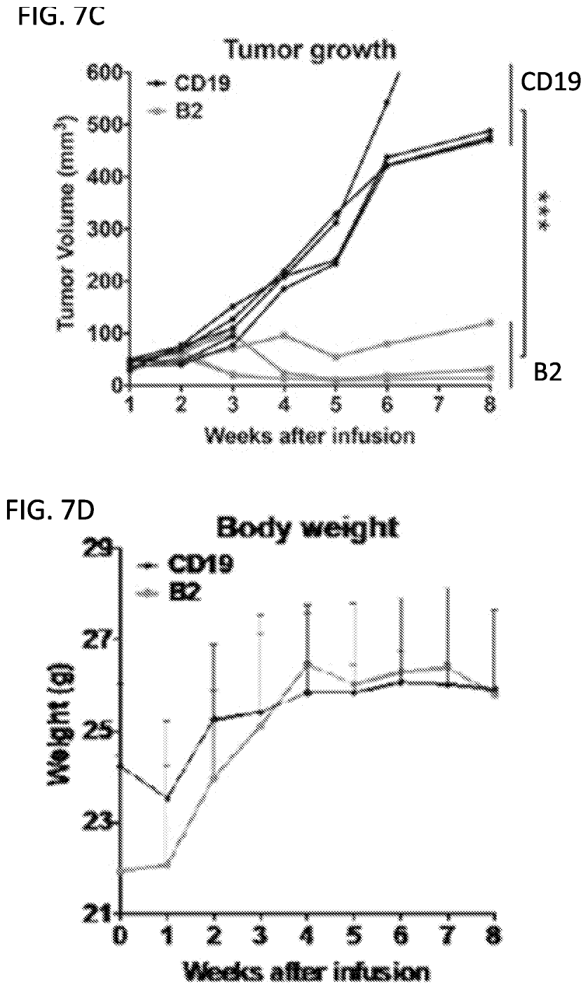

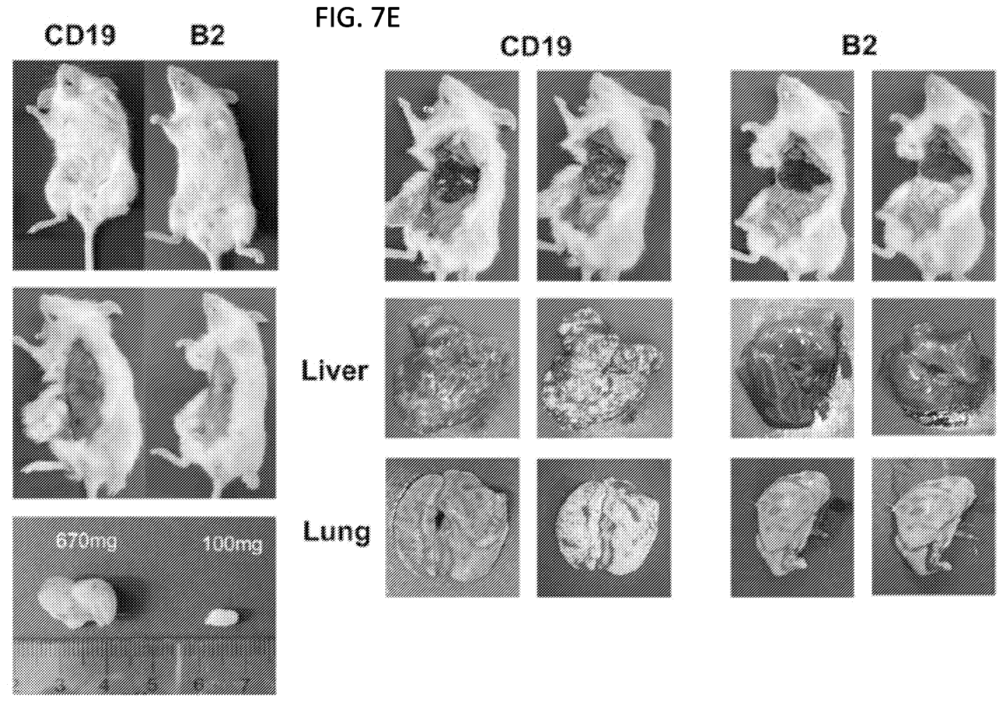

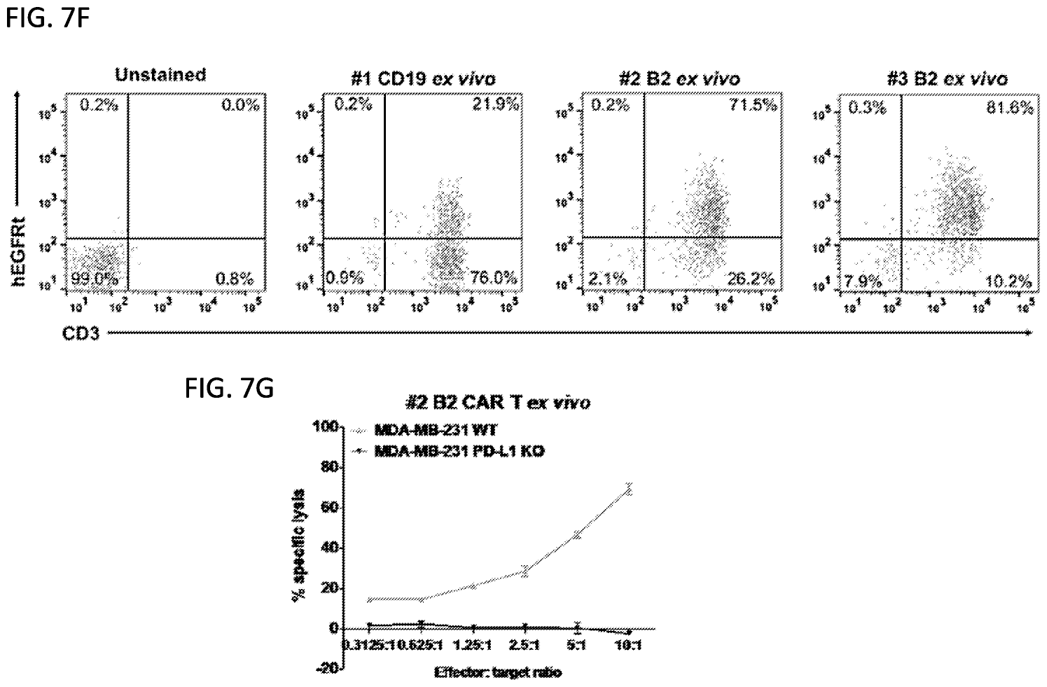

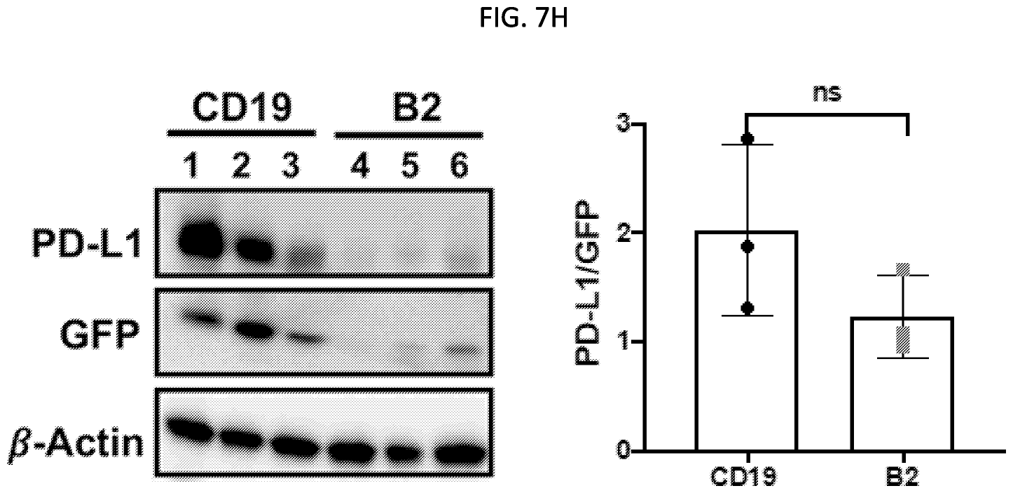

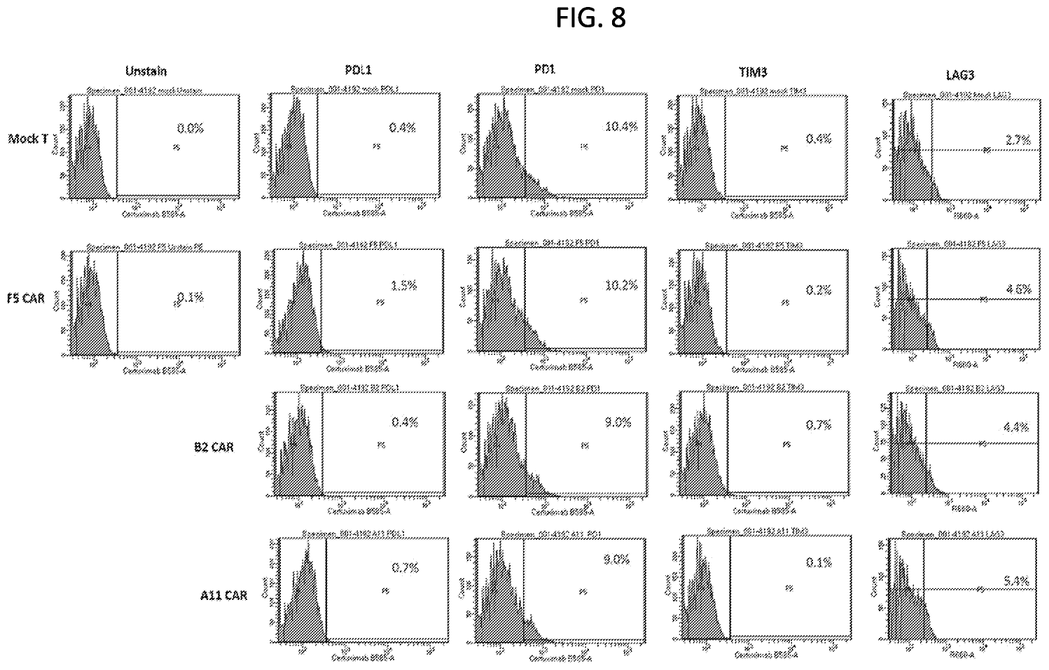

PD-L1 is not only overexpressed on a larger number of malignancies, but also on immune cells in the tumor microenvironment (Sun et al., Immunity 2018; 48(3):434-452). T cells express low levels of endogenous PD-L1, which makes the development of CAR T cells that target PD-L1 complex (Xie et al., Proc Natl Acad Sci USA 2019; 116(16):7624-7631; Qin et al., Biomark Res 2020; 8:19). Antigen exposure of CAR T cells may lead to T cell fratricide and exhaustion, impairing the proliferation and persistence of CAR T cells in vitro and in vivo. For example, Xie et al. reported that camelid V_{H}H-based anti-mouse PD-L1 CAR T cells were found to be self-activated in vitro and PD-L1 proficient CAR T cells could live longer than WT CAR T cells (Xie et al., Proc Natl Acad Sci USA 2019; 116(16):7624-7631). However, the present study did not find an upregulated expression of exhaustion markers such as PD-1, TIM-3, and LAG-3 on the surface of in vitro activated CAR (B2) T cells compared with mock T cells (FIG. 8). CAR (B2) T cells isolated from mouse spleens 3 weeks after infusion still efficiently lysed MDA-MB-231 cells in vitro (FIG. 7G), which may be due to two reasons. First, shark nanobodies have a unique structure and binding curve, which is different from scFv and camelid V_{H}H. B2 may functionally block interaction of PD-L1 to PD-1, inhibiting CAR T exhaustion. Second, in comparison with other public anti-PD-L1 antibodies, shark V_{NAR}B2 does not have a comparably high binding affinity to the antigen. Ghorashian et al. reported a novel CD19 CAR with a lower affinity binder and found that increased immunoreceptor affinity may adversely affect T cell responses (Ghorashian et al., Nat Med 2019; 25(9):1408-1414).

To overcome tumor escape mechanisms and enhance anti-tumor efficacy of CAR T cells, a combination strategy could be used in solid tumor therapy, such as CAR T cells with monoclonal antibodies, small-molecules, or bi-specific CAR T cells targeting different tumor antigens (Pan et al., Cancer Immunol Immunother 2018; 67(10):1621-1634; Hegde et al., J Clin Invest 2016; 126(8):3036-3052). In the in vitro experiments disclosed herein, bi-specific CAR (hYP7−B2) T cells targeting both GPC3 and the tumor microenvironment marker PD-L1 significantly potentiated killing of HCC cells (Hep3B) by CAR (hYP7) T cells, indicating that anti-PD-L1 B2 nanobody is suitable for engineering of bi-specific CAR T cells. It is believed that engineered CAR T cells targeting PD-L1 exhibit dual function. These CAR T cells not only exerted direct killing by recognizing PD-L1, but also blocked interaction of PD-1 to PD-L1 to inhibit T cell exhaustion.

### I. Abbreviations

ACT adoptive cell therapy
ADC antibody-drug conjugate
CAR chimeric antigen receptor
CDR complementarity determining region
CRISPR clustered regularly interspaced short palindromic repeats
E:T effector to target ratio
ECD extracellular domain
FR framework region
GPC3 glypican-3
HCC hepatocellular carcinoma
hEGFRt human epidermal growth factor receptor truncated
HRP horseradish peroxidase
HV hypervariable
IFN interferon
IL interleukin
KO knockout
MHC major histocompatibility complex
MOI multiplicity of infection
NK natural killer
NSG NOD scid gamma
OC ovarian cancer
PBMC peripheral blood mononuclear cells
PD-L1 programmed death ligand 1
PD-1 programmed death 1
PE Pseudomonas exotoxin or phycoerythrin
TIL tumor-infiltrating lymphocytes
TNBC triple negative breast cancer
TNF tumor necrosis factor
VH variable heavy
VL variable light
V_{NAR} variable domain of the immunoglobulin new antigen receptor
WT wild type

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various embodiments, the following explanations of terms are provided:

Administration: To provide or give a subject an agent, such as a polypeptide (for example, a single-domain monoclonal antibody) provided herein, by any effective route. Exemplary routes of administration include, but are not limited to, oral, injection (such as subcutaneous, intramuscular, intradermal, intraperitoneal, intravenous, intra-arterial (including hepatic intra-arterial), intraprostatic, and intratumoral), sublingual, rectal, transdermal, intranasal, vaginal and inhalation routes. In some examples administration is local. In some examples administration is systemic.

Antibody: A polypeptide ligand comprising at least one variable region that recognizes and binds (such as specifically recognizes and specifically binds) an epitope of an antigen, such as a PD-L1 antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and has some functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{NAR} antibodies, camelid V_{H}H antibodies, V_{H} domain antibodies and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by known methods. Monoclonal antibodies include humanized monoclonal antibodies.

A “chimeric antibody” has framework residues from one species, such as human, and CDRs (which generally confer antigen binding) from another species, such as a V_{NAR} that specifically binds a viral antigen.

A “humanized” antibody is an immunoglobulin including a human framework region and one or more CDRs from a non-human (for example a shark, mouse, rabbit, rat, or synthetic) immunoglobulin. The non-human immunoglobulin providing the CDRs is termed a “donor,” and the human immunoglobulin providing the framework is termed an “acceptor.” In one embodiment, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions. Methods of humanizing shark V_{NAR} antibodies has been previously described (Kovalenko et al., J Biol Chem 288(24):17408-17419, 2013).

Antibody-drug conjugate (ADC): A molecule that includes an antibody (or antigen-binding fragment of an antibody, such an anti-PD-L1 antibody provided herein) conjugated to a drug, such as a cytotoxic agent. ADCs can be used to specifically target a drug to particular cells through specific binding of the antibody to a target antigen expressed on the cell surface. Exemplary drugs for use with ADCs include anti-microtubule agents (such as maytansinoids, auristatin E and auristatin F) and interstrand crosslinking agents (for example, pyrrolobenzodiazepines; PBDs). In some cases, the ADC is a bi-specific ADC, which is comprised of two monoclonal antibodies or antigen-fragments thereof, each directed to a different antigen or epitope, conjugated to a drug. In one example, the agent attached to the antibody is IRDye® 700 DX (IR700, Li-cor, Lincoln, NE), which can then be used with near infrared (NIR) light to kill target cells to which the antibody binds (photoimmunotherapy; see for example U.S. Pat. Nos. 8,524,239 and 10,538,590). For example, amino-reactive IR700 can be covalently conjugated to an antibody using the NHS ester of IR700.

Binding affinity: Affinity of an antibody for an antigen (such as such an anti-PD-L1 single-domain antibody provided herein and PD-L1, such as PD-L1 from human, mouse or dog). In one embodiment, affinity is calculated by a modification of the Scatchard method described by Frankel et al., Mol. Immunol., 16:101-106, 1979. In another embodiment, binding affinity is measured by an antigen/antibody dissociation rate. In another embodiment, a high binding affinity is measured by a competition radioimmunoassay. In another embodiment, binding affinity is measured by ELISA. In some embodiments, binding affinity is measured using the Octet system (Creative Biolabs), which is based on bio-layer interferometry (BLI) technology. In other embodiments, Kd is measured using surface plasmon resonance assays using a BIACORES-2000 or a BIACORES-3000 (BIAcore, Inc., Piscataway, N.J.). In other embodiments, antibody affinity is measured by flow cytometry or by surface plasmon reference. An antibody that “specifically binds” an antigen (such as PD-L1, such as human, mouse or canine PD-L1) is an antibody that binds the antigen with high affinity and does not significantly bind other unrelated antigens. In some examples, a monoclonal antibody (such as an anti-PD-L1 single-domain antibody provided herein) specifically binds to a target (for example, human, mouse or canine PD-L1) with an equilibrium constant (Kd) of 50 nM or less, such as 45 nM or less, 40 nM or less, 35 nM or less, 30 nM or less, 25 nM or less, 20 nM or less, 15 nM or less, 10 nM or less, or 5 nM or less.

Bispecific antibody: A recombinant protein that includes antigen-binding fragments of two different monoclonal antibodies, and is thereby capable of binding two different antigens or two different epitopes of the same antigen. Similarly, a multi-specific antibody is a recombinant protein that includes antigen-binding fragments of at least two different monoclonal antibodies, such as two, three or four different monoclonal antibodies.

Breast cancer: A type of cancer that forms in tissues of the breast, usually the ducts and lobules. Types of breast cancer include, for example, ductal carcinoma in situ, invasive ductal carcinoma, triple negative breast cancer (TNBC), inflammatory breast cancer, metastatic breast cancer, medullary carcinoma, tubular carcinoma and mucinous carcinoma. TNBC refers to a type of breast cancer in which the cancer cells do not express estrogen receptors, progesterone receptors or significant levels of HER2/neu protein. TNBC is also called ER-negative PR-negative HER2/neu-negative breast cancer.

Chemotherapeutic agent: Any chemical agent with therapeutic usefulness in the treatment of diseases characterized by abnormal cell growth. Such diseases include tumors, neoplasms, and cancer. In one embodiment, a chemotherapeutic agent is an agent of use in treating a PD-L1-positive tumor. In one embodiment, a chemotherapeutic agent is a radioactive compound. Exemplary chemotherapeutic agents that can be used with the methods provided herein are disclosed in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds.): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer. One example is the administration of an antibody that binds PD-L1 (such as one provided herein) used in combination with a radioactive or chemical compound. In one example, a chemotherapeutic agent is a biologic, such as a therapeutic antibody (e.g., therapeutic monoclonal antibody), such as an anti-PD-L1 antibody provided herein, as well as other anti-cancer antibodies, such as anti-PD-1 or anti-GPC3, anti-CTLA4 (e.g., ipilimumab), anti-EGFR (e.g., cetuximab), anti-VEGF (e.g., bevacizumab), or combinations thereof (e.g., anti-PD-1 and anti-CTLA-4).

Chimeric antigen receptor (CAR): A chimeric molecule that includes an antigen-binding portion (such as single-domain antibody) and a signaling domain, such as a signaling domain from a T cell receptor (for example, CD3ζ). Typically, CARs are comprised of an antigen-binding moiety, a transmembrane domain and an endodomain. The endodomain typically includes a signaling chain having an immunoreceptor tyrosine-based activation motif (ITAM), such as CD3ζ or FcεRIγ. In some instances, the endodomain further includes the intracellular portion of at least one additional co-stimulatory domain, such as CD28, 4-1BB (CD137), ICOS, OX40 (CD134), CD27 and/or DAP10. In some examples, the CAR is multispecific (such as bispecific) or bicistronic. A multispecific CAR is a single CAR molecule comprised of at least two antigen-binding domains (such as scFvs and/or single-domain antibodies) that each bind a different antigen or a different epitope on the same antigen (see, for example, US 2018/0230225). For example, a bispecific CAR refers to a single CAR molecule having two antigen-binding domains that each bind a different antigen. A bicistronic CAR refers to two complete CAR molecules, each containing an antigen-binding moiety that binds a different antigen. In some cases, a bicistronic CAR construct expresses two complete CAR molecules that are linked by a cleavage linker. T cells or NK cells (or other immune cells, such as macrophages) expressing a bispecific or bicistronic CAR can bind cells that express both of the antigens to which the binding moieties are directed (see, for example, Qin et al., Blood 130:810, 2017; and WO/2018/213337).

Complementarity determining region (CDR): A region of hypervariable amino acid sequence that defines the binding affinity and specificity of an antibody. The shark V_{NAR} single-domain antibodies disclosed herein include two CDRs (CDR1 and CDR3). Shark V_{NAR} antibodies further include two hypervariable regions, referred to as HV2 and HV4.

Conjugate: In the context of the present disclosure, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, carbohydrate or recombinant virus. An antibody conjugate is often referred to as an “immunoconjugate.” When the conjugate includes an antibody linked to a drug (e.g., a cytotoxic agent), the conjugate is often referred to as an “antibody-drug conjugate” or “ADC.” Other antibody conjugates include, for example, multi-specific (such as bispecific or trispecific) antibodies and chimeric antigen receptors (CARs).

Conservative variant: “Conservative” amino acid substitutions are those substitutions that do not substantially affect or decrease the affinity of a protein. For example, a monoclonal antibody that specifically binds a target antigen (such as PD-L1) can include at most about 1, at most about 2, at most about 5, at most about 10, or at most about 15 conservative substitutions and specifically bind the target antigen. The term “conservative variant” also includes the use of a substituted amino acid in place of an unsubstituted parent amino acid, provided that the antibody specifically binds the target antigen. Non-conservative substitutions are those that reduce an activity or binding to the target antigen.

Conservative amino acid substitution tables providing functionally similar amino acids are well known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Contacting: Placement in direct physical association; includes both in solid and liquid form.

Cytotoxic agent: Any drug or compound that kills cells.

Cytotoxicity: The toxicity of a molecule, such as an immunotoxin, to the cells intended to be targeted, as opposed to the cells of the rest of an organism. In one embodiment, in contrast, the term “toxicity” refers to toxicity of an immunotoxin to cells other than those that are the cells intended to be targeted by the targeting moiety of the immunotoxin, and the term “animal toxicity” refers to toxicity of the immunotoxin to an animal by toxicity of the immunotoxin to cells other than those intended to be targeted by the immunotoxin.

Diagnostic Imaging: Coupling antibodies and their derivatives with positron emitting radionuclides for positron emission tomography (PET) is a process often referred to as immunoPET. While full length antibodies can make good immunoPET agents, their biological half-life necessitates waiting several days prior to imaging, resulting in an increase in non-target radiation doses. Smaller, single domain antibodies (such as shark V_{NAR}) have biological half-lives amenable to same day imaging.

Drug: Any compound used to treat, ameliorate or prevent a disease or condition in a subject. In some embodiments herein, the drug is an anti-tumor agent.

Effector molecule: The portion of an antibody conjugate (or immunoconjugate) that is intended to have a desired effect on a cell to which the conjugate is targeted. Effector molecules are also known as effector moieties, therapeutic agents, diagnostic agents, or similar terms. Therapeutic agents (or drugs) include such compounds as small molecules, nucleic acids, proteins, peptides, amino acids or derivatives, glycoproteins, radioisotopes, lipids, nanoparticles, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the effector molecule can be contained within an encapsulation system, such as a nanoparticle, liposome or micelle, which is conjugated to the antibody. Encapsulation shields the effector molecule from direct exposure to the circulatory system. Means of preparing liposomes attached to antibodies are well known (see, for example, U.S. Pat. No. 4,957,735; and Connor et al., Pharm Ther 28:341-365, 1985). Diagnostic agents or moieties include radioisotopes and other detectable labels (e.g., fluorophores, chemiluminescent agents, and enzymes). Radioactive isotopes include ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I.

Epitope: An antigenic determinant. These are particular chemical groups or peptide sequences on a molecule that are antigenic, meaning that they elicit a specific immune response. An antibody specifically binds a particular antigenic epitope on a polypeptide.

Framework region: Amino acid sequences interposed between CDRs. The framework regions serve to hold the CDRs in an appropriate orientation for antigen binding.

Fusion protein: A protein comprising at least a portion of two different (heterologous) proteins. In some embodiments, the fusion protein includes a polypeptide (such as a single-domain monoclonal antibody) disclosed herein and a heterologous protein, such as an Fc protein.

Hepatocellular carcinoma (HCC): A primary malignancy of the liver typically occurring in patients with inflammatory livers resulting from viral hepatitis, liver toxins or hepatic cirrhosis (often caused by alcoholism). HCC is also called malignant hepatoma.

Heterologous: Originating from a separate genetic source or species. For example, a shark antibody is heterologous to a human Fc protein.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4⁺ response or a CD8⁺ response. In another embodiment, the response is a B cell response, and results in the production of antigen-specific antibodies.

Immunoconjugate: A covalent linkage of an effector molecule to an antibody or functional fragment thereof. The effector molecule can be, for example, a detectable label, a photon absorber (such as IR700), or a toxin (to form an immunotoxin, such as an immunotoxin comprising Pseudomonas exotoxin or a variant thereof). Specific, non-limiting examples of toxins include, but are not limited to, abrin, ricin, Pseudomonas exotoxin (PE, such as PE35, PE37, PE38, and PE40), diphtheria toxin (DT), botulinum toxin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain Ia of PE and the B chain of DT) and replacing it with a different targeting moiety, such as an antibody. In one embodiment, an antibody is joined to an effector molecule. In another embodiment, an antibody joined to an effector molecule is further joined to a lipid or other molecule, such as to increase its half-life in the body. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because immunoconjugates were originally prepared from two molecules with separate functionalities, such as an antibody and an effector molecule, they are also sometimes referred to as “chimeric molecules.” The term “chimeric molecule,” as used herein, therefore refers to a targeting moiety, such as a ligand or an antibody, conjugated (coupled) to an effector molecule. The term “conjugated” or “linked” refers to making two polypeptides into one contiguous polypeptide molecule.

Immunoglobulin new antigen receptor (IgNAR) antibody: One of the three isotypes of immunoglobulin molecules produced by cartilaginous fish. IgNAR antibodies are homodimers of one variable new antigen receptor (V_{NAR}) domain and five constant new antigen receptor (C_{NAR}) domains (Roux et al., Proc Natl Acad Sci USA 95:11804-11809, 1998). IgNAR antibodies are a major component of the immune system of cartilaginous fish.

Immunoliposome: A liposome with antibodies or antibody fragments conjugated to its surface. Immunoliposomes can carry cytotoxic agents or other drugs to antibody-targeted cells, such as tumor cells.

Isolated: An “isolated” biological component, such as a nucleic acid, protein (including antibodies) or organelle, has been substantially separated or purified away from other biological components in the environment (such as a cell) in which the component occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

Linker: In some cases, a linker is a peptide within an antibody binding fragment (such as an Fv fragment) which serves to indirectly bond the variable heavy chain to the variable light chain. “Linker” can also refer to a peptide serving to link a targeting moiety, such as an antibody, to an effector molecule, such as a cytotoxin or a detectable label. The terms “conjugating,” “joining,” “bonding” or “linking” refer to making two polypeptides into one contiguous polypeptide molecule, or to covalently attaching a radionuclide, drug or other molecule to a polypeptide, such as an antibody or antibody fragment. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

Liver cancer: Any type of cancer occurring in liver tissue. The most common type of liver cancer is hepatocellular carcinoma (HCC), which develops in hepatocytes. Other types of liver cancer include cholangiocarcinoma, which develops in the bile ducts; liver angiosarcoma, which is a rare form of liver cancer that begins in the blood vessels of the liver; and hepatoblastoma, which is a very rare type of liver cancer found most often in children.

Neoplasia, malignancy, cancer or tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.”

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013,), describes compositions and formulations suitable for pharmaceutical delivery of the antibodies and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Photoimmunotherapy: A targeted therapy that utilizes an antigen-specific antibody-photoabsorber conjugate that can be activated by near-infrared light to kill targeted cells. The photon absorber is typically based on phthalocyanine dye, such as a near infrared (NIR) phthalocyanine dye (for example, IRDye® 700DX, also know known as IR700). The antibody (for example, a PD-L1-specific antibody) binds to the appropriate cell surface antigen (e.g., PD-L1) and the photo-activatable dye induces lethal damage to cell membranes after NIR-light exposure. NIR-light exposure (e.g., 690 nm) induces highly selective, necrotic cell death within minutes without damage to adjoining cells (see, for example, U.S. Application No. 2018/0236076). Thus, such methods can be used to kill tumor cells expressing PD-L1.

Polypeptide: A polymer in which the monomers are amino acid residues joined together through amide bonds. When the amino acids are alpha-amino acids, either the L-optical isomer or the D-optical isomer can be used. The terms “polypeptide” and “protein” are used herein interchangeably and include standard amino acid sequences as well as modified sequences, such as glycoproteins. The term “polypeptide” is specifically intended to cover naturally occurring proteins, as well as proteins that are recombinantly or synthetically produced. In the context of the present disclosure, a “polypeptide” is any protein or polypeptide (natural, recombinant or synthetic) that is capable of specific binding to a target antigen, such as PD-L1 or portion thereof. Thus, the polypeptides disclosed herein include at least one, such as one, two or three, CDR sequences that mediate specific binding to the target antigen. In some embodiments, the polypeptide is a single-domain monoclonal antibody, such as a shark V_{NAR} single-domain monoclonal antibody, isolated from a phage display library, or a modified form thereof (such as a humanized or chimeric single-domain monoclonal antibody). In other embodiments, the polypeptide comprises fibronectin (adectin), albumin, protein A (affibody), a peptide aptamer, an affimer, an affitin, an anticalin, or another antibody mimetic (see, e.g., Yu et al., Annu Rev Anal Chem 10(1): 293-320, 2017; Ta and McNaughton, Future Med Chem 9(12): 1301-1304, 2017; Koutsoumpeli et al., Anal Chem 89(5): 3051-3058, 2017), or a similar protein in which one or more CDR sequences have been incorporated to confer specific binding to the target antigen.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in viral load. “Ameliorating” refers to the reduction in the number or severity of signs or symptoms of a disease.

Programmed death ligand 1 (PD-L1): An immune inhibitory receptor ligand expressed by hematopoietic and non-hematopoietic cells, such as T cells, B cells and several different tumor types. PD-L1 is a type I transmembrane protein with immunoglobulin V-like and C-like domains. Interaction of PD-L1 with its receptor inhibits T-cell activation and cytokine production. During infection or inflammation of normal tissue, this interaction is important for preventing autoimmunity by maintaining homeostasis of the immune response. In tumor microenvironments, this interaction provides an immune escape for tumor cells through cytotoxic T-cell inactivation. PD-L1 is also known as CD274, B7-H and B7H1. Nucleic acid and protein sequences of PD-L1 are publicly available, such as under NCBI Gene ID 29126. An exemplary mouse PD-L1 is available under GenBank® Accession No. ADK70950.1. An exemplary canine PD-L1 is available under GenBank® Accession No. BAO74172.1. An exemplary human PD-L1 is available under GenBank® Accession No. Q9NZQ7.1. Exemplary human, mouse and canine PD-L1 extracellular domains (ECDs) are set forth herein as SEQ ID NOs: 29, 30, and 31, respectively.

PD-L1-positive cancer: A cancer that expresses PD-L1 or can be induced to express PD-L1, such as by IFNγ. Examples of PDL-1-positive cancers include, but are not limited to liver cancer (such as hepatocellular carcinoma), breast cancer (such as triple negative breast cancer), pancreatic cancer, melanoma, non-small cell lung cancer (NSCLC), renal cell carcinoma, bladder cancer, head and neck squamous cell carcinoma (HNSCC), gastric cancer, urothelial carcinoma and Merkel cell carcinoma. Thus, such cancers can be detected and treated with the disclosed compositions and methods.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Sample (or biological sample): A biological specimen containing genomic DNA, RNA (including mRNA), protein, or combinations thereof, which can be obtained from a subject. Examples include, but are not limited to, blood, serum, urine, semen, sputum, saliva, mucus, nasal wash, tissue, cells, tissue biopsy, fine needle aspirate, surgical specimen, feces, cerebral spinal fluid (CSF), bronchoalveolar lavage (BAL) fluid, nasopharyngeal samples, oropharyngeal samples, and autopsy material. In one example, a sample is a tumor biopsy or fine needle aspirate.

Sequence Identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are well known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of an antibody that specifically binds a target antigen or a fragment thereof are typically characterized by possession of at least about 75%, for example at least about 80%, 90%, 95%, 96%, 97%, 98% or 99% sequence identity counted over the full length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill will appreciate that these sequence identity ranges are provided for guidance only; it is entirely possible that strongly significant homologs could be obtained that fall outside of the ranges provided.

Small molecule: A molecule, typically with a molecular weight less than about 1000 Daltons, or in some embodiments, less than about 500 Daltons, wherein the molecule is capable of modulating, to some measurable extent, an activity of a target molecule.

Subject: Living multi-cellular vertebrate organisms, a category that includes both human and veterinary subjects, including human and non-human mammals such as pigs, mice, rats, rabbits, sheep, horses, cows, dogs, cats and non-human primates.

Synthetic: Produced by artificial means in a laboratory, for example a synthetic nucleic acid or protein (for example, an antibody) can be chemically synthesized in a laboratory.

Therapeutically effective amount: The amount of agent, such as a polypeptide (e.g., a single-domain monoclonal antibody specific for PD-L1 provided herein), that is sufficient to prevent, treat (including prophylaxis), reduce and/or ameliorate one or more symptoms and/or underlying causes of a disease or disorder, for example to prevent, inhibit, and/or treat a PD-L1-positive cancer. In one embodiment, a therapeutically effective amount is the amount necessary to eliminate, reduce the size, or prevent metastasis of a tumor, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment. When administered to a subject, a dosage will generally be used that will achieve target tissue concentrations (for example, in tumors) that has been shown to achieve a desired in vitro effect.

A therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Toxin: An agent that directly or indirectly inhibits the growth of and/or kills cells. Toxins include, for example, Pseudomonas exotoxin (PE, such as PE35, PE37, PE38 and PE40), diphtheria toxin (DT), botulinum toxin, abrin, ricin, saporin, restrictocin or gelonin, or modified toxins thereof. For example, PE and DT are highly toxic compounds that typically bring about death through liver toxicity. PE and DT, however, can be modified into a form for use as an immunotoxin by removing the native targeting component of the toxin (such as domain Ia of PE or the B chain of DT) and replacing it with a different targeting moiety, such as an antibody.

Variable new antigen receptor (V_{NAR}): The single variable domain of the immunoglobulin new antigen receptor (IgNAR) antibody found in cartilaginous fish. V_{NAR} antibodies are comprised of only two CDRs (CDR1 and CDR3), but also contain two other hypervariable (HV) regions, referred to as the HV2 and HV4 regions. The CDRs and HV regions are surrounded by framework regions (FR) in the following N-terminal to C-terminal order: FR1-CDR1-FR2-HV2-FR3a-HV4-FR3b-CDR3-FR4.

The V_{NAR} domain, like other variable domains, has an immunoglobulin fold that contains β sheets held together by two canonical cysteine residues. In addition to the cysteines found in framework region (FR) 1 and 3b, the CDR3 can have one or two additional cysteines that form disulfide bonds with CDR1 or other framework regions. IgNAR are classified into four types based on the number and positioning of non-canonical cysteines in the V_{NAR} domain. Type I V_{NAR} domains contain two cysteine residues in CDR3 that form two extra disulfide bonds with FR2 and FR4. Type II V_{NAR} domains have one non-canonical cysteine in CDR3 that forms a disulfide bond with a non-canonical cysteine in CDR1. Type III V_{NAR} domains form a disulfide bond in CDR3 and FR2, and type IV domains have no additional disulfide bonds. While type I V_{NAR} usually have flatter antigen binding regions and CDR3 regions that average 21 amino acids long, type II are usually shorter with an average of 15 amino acids and have a protruding CDR3 that enables binding to pockets and grooves (Barelle et al., Adv Exp Med Biol 655:49-62, 2009). The canonical CDR2 loop in classical IgG is missing in V_{NAR} and is replaced with a short stretch of highly diverse amino acids, termed hypervariable region 2 (HV2) (Stanfield et al., Science 305:1770-1773, 2004). Additionally, there is a second hypervariable region, named HV4, which is inserted in the middle of FR3, therefore breaking FR3 into FR3a and FR3b.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements. In some embodiments, the vector is a virus vector, such as an AAV vector or lentivirus vector.

### III. Single-Domain Antibodies Specific for PD-L1

A single-chain antibody variable fragment (scFv), which consists of variable heavy (V_{H}) and variable light (V_{L}) (Oishi et al., Hum Mol Genet 2002; 11(23):2951-2960) chains connected by a flexible linker (such as (Gly₄Ser)₃), usually serves as the antigen recognition region of a CAR construct. However, proper folding of artificially engineered scFv can alter the specificity and affinity of the CAR for its target antigen (Chailyan et al., FEBS J 2011; 278(16):2858-2866). In contrast, the antigen binding domain of natural single-domain antibodies (heavy chain only) from camelid (V_{H}H) (Hamers-Casterman et al., Nature 1993; 363(6428):446-448) and shark (V_{NAR}) (Flajnik et al., Nat Rev Genet 2010; 11(1):47-59) have beneficial properties for the engineering of CARs. They are small in size (12-15 kDa), easily expressed, and capable of binding concave and hidden epitopes that are not accessible to conventional antibodies (Muyldermans et al., Annu Rev Biochem 2013; 82:775-797). Shark V_{NAR} antibodies have unique features that are quite different from camel V_{H}H antibodies, such as a large diversity in the number and positions of cysteines, and are evolutionally derived from an ancient single domain antibody that functions as a variable domain in both B cell and T cell receptors (Criscitiello et al., Proc Natl Acad Sci USA 2006; 103(13):5036-5041; English et al., Antib Ther 2020; 3(1):1-9). A shark V_{NAR} phage-displayed library was previously constructed (Feng et al., Antib Ther 2019; 2(1):1-11). The present disclosure describes the reconstruction of a semi-synthetic shark V_{NAR} phage library in which the V_{NAR} antibodies have a randomized complementarity determining region 3 (CDR3) of 18 amino acids in length (Brahmer et al., N Engl J Med 2015; 373(2):123-135). Panning of the reconstructed library led to the identification of 12 PD-L1 binders that are cross-reactive with human and mouse PD-L1, and in some instances, also bind canine PD-L1. This is the first report of human and mouse cross-reactive PD-L1 monoclonal antibodies, as well as the first disclosure of PD-L1-specific single-domain monoclonal antibodies.

The amino acid sequences of 12 PD-L1-specific single-domain V_{NAR} antibodies selected from the re-engineered shark V_{NAR} phage library are provided below (and set forth herein as SEQ ID NOs: 1-12). Shark V_{NAR} are comprised of the following regions (N-terminal to C-terminal): FR1-CDR1-FR2-HV2-FR3a-HV4-FR3b-CDR3-FR4. CDR and HV regions were determined using shark V_{NAR} annotation (italics) as described by Stanfield et al. (Science 305:1770-1773, 2004) and Fennell et al. (J Mol Biol 400:155-170, 2010). CDRs were also determined using IMGT (bold) and Kabat (underline). The positions of each CDR and HV region are shown in Tables 1 and 2.

Shark V_{NAR} B2
(SEQ ID NO: 1)
ARVDQTPRSVTKETGESLTINCVLRDSSYALGSTY
WYRKKSGSTNEESISKGGRYVETVNSGSKSFSLRI
NDLTVEDSGTYRCKYTSRLRREGPLSWDGNTV
YGGGTVVTVN
Shark V_{NAR} F5
(SEQ ID NO: 2)
ARVDQTPRSVTKETGESLTINCVLRDTNYALGSTY
WYRKKLGSTNEESISKGGRYVETVNSGSKSFSLRI
NDLTVEDSGTYRCKNLGHPMYIAGAICRPLPI
YGGGTVVTVN
Shark V_{NAR} A11
(SEQ ID NO: 3)
ARVDQTPRSVTKETGESLTINCVLRDTSYALGSTY
WYRKKSGSTNEESISKGGRYVETVNSGSKSFSLRI
NDLTVEDSGTYRCKFKSNTEFLNPLTVSMSTI
YGGGTVVTVN
Shark V_{NAR} A3
(SEQ ID NO: 4)
ARVDQTPQTITKETGESLTINCVLRDSNYALGSTY
WYRKKSGSTNEESISKGGRYVETVNSGSKSFSLRI
NDLTVEDSGTYRCKTQKAIPCNMNGYVCGVTI
YGGGTVVTVN
Shark V_{NAR} A9
(SEQ ID NO: 5)
ARVDQTPQTITKETGESLTINCVLRDTNYALGSTY
WYRKKLGSTNEESISKGGRYVETVNSGSKSFSLRI
NDLTVEDSGTYRCKNTSPWLTYSPWTVSGQTS
YGGGTVVTVN
Shark V_{NAR} A2
(SEQ ID NO: 6)
ARVDQTPRSVTKETGESLTINCVLRDTSYALGSTY
WYRKKLGSTNEESISKGGRYVETVNSGSKTFSLRI
NDLTVEDSGTYRCKVQTTWSYLSPYKIEFATV
YGGGTVVTVN
Shark V_{NAR} A10
(SEQ ID NO: 7)
ARVDQTPRSVTKETGESLTINCVLRDTNYALGSTY
WYRKKLGSTNEESISKGGRYVETVNSGSKSFSLRI
NDLTVEDSGTYRCKSTTPWNNLTSFTSERVTI
YGGGTVVTVN
Shark V_{NAR} A7
(SEQ ID NO: 8)
ARVDQTPRSVTKETGESLTINCVLRDTSYALGSTY
WYRKKLGSTNEESISKGGRYVETVNSGSKSFSLRI
NDLTVEDSGTYRCKM
NQRYPCDNNSWWSLYCGTTVYGGGTVVTVN
Shark V_{NAR} A6
(SEQ ID NO: 9)
ARVDQTPQTITKETGESLTINCVLRDTSYALGSTY
WYRKKSGSTNEESISKGGRYVETVNSGSKSFSLRI
NDLRVEDSGTYRCKY
SSSWQKATEGVWEAMTEYGGGTVVTVN
Shark V_{NAR} C4
(SEQ ID NO: 10)
ARVDQTPRSVTKETGESLTINCVLRDTNYALGSTY
WYRKKSGSTNEESISKGGRYVETVNSGSKSFSLRI
NDLTVEDSGTYRCKQ
SASLWIPNLLRWVPVISYGGGTVVTVN
Shark V_{NAR} A1
(SEQ ID NO: 11)
ARVDQTPRSVTKETGESLTINCVLRDTNYALGSTY
WYRKKSGSTNEESISKGGRYVETVNSGSKSFSLRI
NDLTVEDSGTYRCKT
SSKPLLVSHNVWSAWTEYGGGTVVTVN
Shark V_{NAR} D12
(SEQ ID NO: 12)
ARVDQTPRSVTKETGESLTINCVLRDTNYALGSTY
WYRKKSGSTNEESISKGGRYVETVNSGSKSFSLRI
NDLTVEDSGTYRCKH
QSSWRRQAPRVMEMQTLYGGGTVVTVN

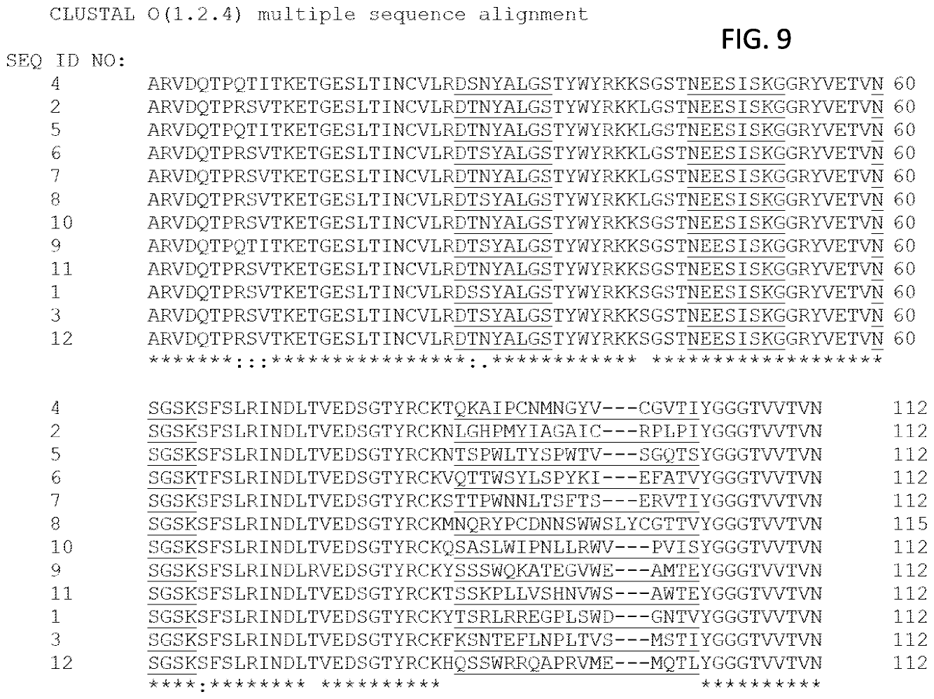

FIG. 9 provides an amino acid sequence alignment of the 12 V_{NAR} antibodies. In the figure, the position is of the CDR1, HV2, HV4 and CDR3, using shark V_{NAR} annotation, are underlined. Based on this alignment, the following consensus CDR1, HV2 and HV4 sequences were determined:

CDR1 =
(SEQ ID NO: 14)
DX₁X₂YALGST,
where X₁ = S or T and X₂ = N or S
HV2 =
(SEQ ID NO: 15)
NEESISKG
HV4 =
(SEQ ID NO: 16)
NSGSK

TABLE 1
Positions of the CDRs and HV regions
using shark V_{NAR} annotation

| | SEQ ID | | | | |
|---|---|---|---|---|---|
| V_{NAR} | NO: | CDR1 | HV2 | HV4 | CDR3 |
| B2 | 1 | 26-33 | 45-52 | 60-64 | 86-102 |
| F5 | 2 | 26-33 | 45-52 | 60-64 | 86-102 |
| A11 | 3 | 26-33 | 45-52 | 60-64 | 86-102 |
| A3 | 4 | 26-33 | 45-52 | 60-64 | 86-102 |
| A9 | 5 | 26-33 | 45-52 | 60-64 | 86-102 |
| A2 | 6 | 26-33 | 45-52 | 60-64 | 86-102 |
| A10 | 7 | 26-33 | 45-52 | 60-64 | 86-102 |
| A7 | 8 | 26-33 | 45-52 | 60-64 | 86-105 |
| A6 | 9 | 26-33 | 45-52 | 60-64 | 86-102 |
| C4 | 10 | 26-33 | 45-52 | 60-64 | 86-102 |
| A1 | 11 | 26-33 | 45-52 | 60-64 | 86-102 |
| D12 | 12 | 26-33 | 45-52 | 60-64 | 86-102 |

TABLE 2
Positions of the CDRs using IMGT and Kabat annotation

| | SEQ ID | | | | |
|---|---|---|---|---|---|
| V_{NAR} | NO: | Annotation | CDR1 | CDR2 | CDR3 |
| B2 | 1 | IMGT | 26-33 | 45-49 | 84-102 |
| B2 | 1 | Kabat | 22-35 | 45-52 | 86-102 |
| F5 | 2 | IMGT | 26-33 | 45-49 | 84-102 |
| F5 | 2 | Kabat | 22-35 | 45-52 | 86-102 |
| A11 | 3 | IMGT | 26-33 | 45-49 | 84-102 |
| A11 | 3 | Kabat | 22-35 | 45-52 | 86-102 |
| A3 | 4 | IMGT | 26-33 | 45-49 | 84-102 |
| A3 | 4 | Kabat | 22-35 | 45-52 | 86-102 |
| A9 | 5 | IMGT | 26-33 | 45-49 | 84-102 |
| A9 | 5 | Kabat | 22-35 | 45-52 | 86-102 |
| A2 | 6 | IMGT | 26-33 | 45-49 | 84-102 |
| A2 | 6 | Kabat | 22-35 | 45-52 | 86-102 |
| A10 | 7 | IMGT | 26-33 | 45-49 | 84-102 |
| A10 | 7 | Kabat | 22-35 | 45-52 | 86-102 |
| A7 | 8 | IMGT | 26-33 | 45-49 | 84-105 |
| A7 | 8 | Kabat | 22-35 | 45-52 | 86-105 |
| A6 | 9 | IMGT | 26-33 | 45-49 | 84-102 |
| A6 | 9 | Kabat | 22-35 | 45-52 | 86-102 |
| C4 | 10 | IMGT | 26-33 | 45-49 | 84-102 |
| C4 | 10 | Kabat | 22-35 | 45-52 | 86-102 |
| A1 | 11 | IMGT | 26-33 | 45-49 | 84-102 |
| A1 | 11 | Kabat | 22-35 | 45-52 | 86-102 |
| D12 | 12 | IMGT | 26-33 | 45-49 | 84-102 |
| D12 | 12 | Kabat | 22-35 | 45-52 | 86-102 |

Provided herein are polypeptides that bind (for example, specifically bind) PD-L1, such as human, mouse and/or canine PD-L1. In some embodiments, the polypeptide (for example, single-domain monoclonal antibody) includes at least a portion of the amino acid sequence set forth herein as any one of SEQ ID NOs: 1-12, such as one or more (such as one, two or three) CDR sequences and/or one or two hypervariable regions from any one of antibodies B2, F5, A11, A3, A9, A2, A10, A7, A6, C4, A1 or D12 (SEQ ID NOs: 1-12, respectively), as determined using any CDR numbering scheme (such as IMGT, Kabat, Paratome or Chothia, or any combination thereof; or using the CDR/HV annotation described in Stanfield et al. 2004 and/or Fennell et al. 2010 for shark V_{NAR}).

In some embodiments, the polypeptide includes the CDR1 and CDR3 sequences of B2 (SEQ ID NO: 1). In some examples, the CDR1 and CDR3 sequences respectively include residues 26-33 and 86-102, residues 26-33 and 84-102, or residues 22-35 and 86-102 of SEQ ID NO: 1. In some examples, the polypeptide further includes the CDR2 sequence of SEQ ID NO: 1, such as residues 45-49 or residues 45-52 of SEQ ID NO: 1. In specific examples, the polypeptide further includes a HV2 region having the sequence of SEQ ID NO: 15 and/or a HV4 region having the sequence of SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 1. In particular non-limiting examples, the amino acid sequence of the polypeptide includes or consists of SEQ ID NO: 1.

In some embodiments, the polypeptide includes the CDR1 and CDR3 sequences of F5 (SEQ ID NO: 2). In some examples, the CDR1 and CDR3 sequences respectively include residues 26-33 and 86-102, residues 26-33 and 84-102, or residues 22-35 and 86-102 of SEQ ID NO: 2. In some examples, the polypeptide further includes the CDR2 sequence of SEQ ID NO: 2, such as residues 45-49 or residues 45-52 of SEQ ID NO: 2. In specific examples, the polypeptide further includes a HV2 region having the sequence of SEQ ID NO: 15 and/or a HV4 region having the sequence of SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 2. In particular non-limiting examples, the amino acid sequence of the polypeptide includes or consists of SEQ ID NO: 2.

In some embodiments, the polypeptide includes the CDR1 and CDR3 sequences of A11 (SEQ ID NO: 3). In some examples, the CDR1 and CDR3 sequences respectively include residues 26-33 and 86-102, residues 26-33 and 84-102, or residues 22-35 and 86-102 of SEQ ID NO: 3. In some examples, the polypeptide further includes the CDR2 sequence of SEQ ID NO: 3, such as residues 45-49 or residues 45-52 of SEQ ID NO: 3. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 3. In particular non-limiting examples, the amino acid sequence of the polypeptide includes or consists of SEQ ID NO: 3.

In some embodiments, the polypeptide includes the CDR1 and CDR3 sequences of A3 (SEQ ID NO: 4). In some examples, the CDR1 and CDR3 sequences respectively comprise residues 26-33 and 86-102, residues 26-33 and 84-102, or residues 22-35 and 86-102 of SEQ ID NO: 4. In some examples, the polypeptide further includes the CDR2 sequence of SEQ ID NO: 4, such as residues 45-49 or residues 45-52 of SEQ ID NO: 4. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 4. In particular non-limiting examples, the amino acid sequence of the polypeptide includes or consists of SEQ ID NO: 4.

In some embodiments, the polypeptide includes the CDR1 and CDR3 sequences of A9 (SEQ ID NO: 5). In some examples, the CDR1 and CDR3 sequences respectively include residues 26-33 and 86-102, residues 26-33 and 84-102, or residues 22-35 and 86-102 of SEQ ID NO: 5. In some examples, the polypeptide further includes the CDR2 sequence of SEQ ID NO: 5, such as residues 45-49 or residues 45-52 of SEQ ID NO: 5. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 5. In particular non-limiting examples, the amino acid sequence of the polypeptide includes or consists of SEQ ID NO: 5.

In some embodiments, the polypeptide includes the CDR1 and CDR3 sequences of A2 (SEQ ID NO: 6). In some example, the CDR1 and CDR3 sequences respectively include residues 26-33 and 86-102, residues 26-33 and 84-102, or residues 22-35 and 86-102 of SEQ ID NO: 6. In some examples, the polypeptide further includes the CDR2 sequence of SEQ ID NO: 6, such as residues 45-49 or residues 45-52 of SEQ ID NO: 6. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 6. In particular non-limiting examples, the amino acid sequence of the polypeptide includes or consists of SEQ ID NO: 6.

In some embodiments, the polypeptide includes the CDR1 and CDR3 sequences of A10 (SEQ ID NO: 7). In some examples, the CDR1 and CDR3 sequences respectively include residues 26-33 and 86-102, residues 26-33 and 84-102, or residues 22-35 and 86-102 of SEQ ID NO: 7. In some examples, the polypeptide further includes the CDR2 sequence of SEQ ID NO: 7, such as residues 45-49 or residues 45-52 of SEQ ID NO: 7. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 7. In particular non-limiting examples, the amino acid sequence of the polypeptide includes or consists of SEQ ID NO: 7.

In some embodiments, the polypeptide includes the CDR1 and CDR3 sequences of A7 (SEQ ID NO: 8). In some examples, the CDR1 and CDR3 sequences respectively include residues 26-33 and 86-105, residues 26-33 and 84-105, or residues 22-35 and 86-105 of SEQ ID NO: 8. In some examples, the polypeptide further includes the CDR2 sequence of SEQ ID NO: 8, such as residues 45-49 or residues 45-52 of SEQ ID NO: 8. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 8. In particular non-limiting examples, the amino acid sequence of the polypeptide includes or consists of SEQ ID NO: 8.

In some embodiments, the polypeptide includes the CDR1 and CDR3 sequences of A6 (SEQ ID NO: 9). In some examples, the CDR1 and CDR3 sequences respectively include residues 26-33 and 86-102, residues 26-33 and 84-102, or residues 22-35 and 86-102 of SEQ ID NO: 9. In some examples, the polypeptide further includes the CDR2 sequence of SEQ ID NO: 9, such as residues 45-49 or residues 45-52 of SEQ ID NO: 9. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 9. In particular non-limiting examples, the amino acid sequence of the polypeptide includes or consists of SEQ ID NO: 9.

In some embodiments, the polypeptide includes the CDR1 and CDR3 sequences of C4 (SEQ ID NO: 10). In some examples, the CDR1 and CDR3 sequences respectively include residues 26-33 and 86-102, residues 26-33 and 84-102, or residues 22-35 and 86-102 of SEQ ID NO: 10. In some examples, the polypeptide further includes the CDR2 sequence of SEQ ID NO: 10, such as residues 45-49 or residues 45-52 of SEQ ID NO: 10. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 10. In particular non-limiting examples, the amino acid sequence of the polypeptide includes or consists of SEQ ID NO: 10.

In some embodiments, the polypeptide includes the CDR1 and CDR3 sequences of A1 (SEQ ID NO: 11). In some examples, the CDR1 and CDR3 sequences respectively include residues 26-33 and 86-102, residues 26-33 and 84-102, or residues 22-35 and 86-102 of SEQ ID NO: 11. In some examples, the polypeptide further includes the CDR2 sequence of SEQ ID NO: 11, such as residues 45-49 or residues 45-52 of SEQ ID NO: 11. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 11. In particular non-limiting examples, the amino acid sequence of the polypeptide includes or consists of SEQ ID NO: 11.

In some embodiments, the polypeptide includes the CDR1 and CDR3 sequence of D12 (SEQ ID NO: 12). In some examples, the CDR1 and CDR3 sequences respectively include residues 26-33 and 86-102, residues 26-33 and 84-102, or residues 22-35 and 86-102 of SEQ ID NO: 12. In some examples, the polypeptide further includes the CDR2 sequence of SEQ ID NO: 12, such as residues 45-49 or residues 45-52 of SEQ ID NO: 12. In specific examples, the polypeptide further includes a HV2 region comprising SEQ ID NO: 15 and/or a HV4 region comprising SEQ ID NO: 16. In some examples, the amino acid sequence of the polypeptide is at least 80%, at least 85%, at least 90%, at least 95%, at least 96%, at least 97%, at least 98% or at least 99% identical to SEQ ID NO: 12. In particular non-limiting examples, the amino acid sequence of the polypeptide includes or consists of SEQ ID NO: 12.

In some embodiments, the polypeptide includes a CDR1, HV2 and CDR3, and the CDR1, HV2 and CDR3 sequences respectively include SEQ ID NO: 14, SEQ ID NO: 15 and residues 86-102 of SEQ ID NO: 1; SEQ ID NO: 14, SEQ ID NO: 15 and residues 86-102 of SEQ ID NO: 2; SEQ ID NO: 14, SEQ ID NO: 15 and residues 86-102 of SEQ ID NO: 3; SEQ ID NO: 14, SEQ ID NO: 15 and residues 86-102 of SEQ ID NO: 4; SEQ ID NO: 14, SEQ ID NO: 15 and residues 86-102 of SEQ ID NO: 5; SEQ ID NO: 14, SEQ ID NO: 15 and residues 86-102 of SEQ ID NO: 6; SEQ ID NO: 14, SEQ ID NO: 15 and residues 86-102 of SEQ ID NO: 7; SEQ ID NO: 14, SEQ ID NO: 15 and residues 86-105 of SEQ ID NO: 8; SEQ ID NO: 14, SEQ ID NO: 15 and residues 86-102 of SEQ ID NO: 9; SEQ ID NO: 14, SEQ ID NO: 15 and residues 86-102 of SEQ ID NO: 10; SEQ ID NO: 14, SEQ ID NO: 15 and residues 86-102 of SEQ ID NO: 11; or SEQ ID NO: 14, SEQ ID NO: 15 and residues 86-102 of SEQ ID NO: 12. In some examples, the polypeptide further includes an HV4 region having the sequence of SEQ ID NO: 16. In some examples, the amino acid sequence of the polypeptide is at least 80%, at least 85%, at least 90%, at least 95%, at least 96%, at least 97%, at least 98% or at least 99% identical to any one of SEQ ID NOs: 1-12. In particular non-limiting examples, the amino acid sequence of the polypeptide includes or consists of any one of SEQ ID NOs: 1-12.

In some embodiments, the polypeptide is a single-domain monoclonal antibody. In some examples, the single-domain monoclonal antibody is a shark V_{NAR} single-domain antibody. In some examples, the single-domain monoclonal antibody is a humanized single-domain monoclonal antibody or a chimeric single-domain monoclonal antibody. In other examples, the polypeptide is a recombinant fibronectin or albumin.

Also provided are fusion proteins that include a PD-L1-specific polypeptide (for example, antibody) disclosed herein and a heterologous protein. In some embodiments, the heterologous protein is an Fc protein or a leucine zipper. A single-domain antibody can be fused to an Fc region to generate a bivalent antibody (e.g., V_{NAR}-Fc). In some examples, the Fc protein is a human Fc protein, such as the human IgG1 Fc. In particular non-limiting examples, the fusion protein includes a single-domain antibody disclosed herein, a hinge region and an Fc domain (such as the human IgG1 Fc domain). In one specific example, the fusion protein further includes a linker, such as a protein linker, such as an Ala-Ala-Ala linker located between the single-domain monoclonal antibody and the hinge region.

Also provided herein are chimeric antigen receptors (CARs) that include a polypeptide (such as a single-domain monoclonal antibody) disclosed herein. In some embodiments, the CAR further includes a hinge region, a transmembrane domain, a costimulatory signaling moiety, a signaling domain, or any combination thereof. In specific non-limiting examples, the hinge region includes a CD8α hinge region, the transmembrane domain includes a CD8α transmembrane domain, the costimulatory signaling moiety includes a 4-1BB signaling moiety and/or the signaling domain includes a CD3ζ signaling domain.

Also provided herein are PD-L1-specific polypeptides (for example, antibodies) modified to enable their use with a universal CAR system. In some embodiments, the PD-L1-specific polypeptide is fused to one component of a specific binding pair. In some examples, the antibody is fused to a leucine zipper or biotin.

Further provided are cells expressing a PD-L1-specific CAR. In some examples, the cell is an immune cell, such as a T lymphocyte, for example a CTL, a natural killer cell, a macrophage or an induced pluripotent stem cell. In some examples, the immune cells are allogeneic cells, such as allogeneic cells obtained from a healthy donor. In some examples, the immune cell further expresses a CAR that specifically binds glypican-3 (GPC3). In specific non-limiting examples, the T cells are genetically modified to express the CAR and optionally to disrupt expression of the endogenous TCR. CARs and CAR-expressing cells are further described in section IV.

Also provided herein are immunoconjugates that include a polypeptide (for example, single-domain antibody) disclosed herein and an effector molecule. In some embodiments, the effector molecule is a toxin, such as, but not limited to, Pseudomonas exotoxin or a variant thereof, such as PE38. In other embodiments, the effector molecule is a detectable label, such as, but not limited to, a fluorophore, an enzyme or a radioisotope. In other embodiments, the effector molecule is a photon absorber, such as IR700. Immunoconjugates comprising a photon absorber can be used for photoimmunotherapy or in vivo diagnostic imaging. Immunoconjugates are further described in section V.

Further provided herein are antibody-drug conjugates (ADCs) that include a drug conjugated to a polypeptide (for example, single-domain antibody) disclosed herein. In some embodiments, the drug is a small molecule, for example an anti-cancer agent, anti-microtubule agent, an anti-mitotic agent and/or a cytotoxic agent. ADCs are further described in section VI.

Also provided herein are multi-specific antibodies that include a polypeptide (for example, single-domain antibody) disclosed herein and at least one additional monoclonal antibody or antigen-binding fragment thereof. In some embodiments, the multi-specific antibody is a bispecific antibody. In other embodiments, the multi-specific antibody is a trispecific antibody. Multi-specific antibodies are further described in section VII.

Further provided herein are antibody-nanoparticle conjugates that include a nanoparticle conjugated to a polypeptide (for example, single-domain antibody) disclosed herein. In some embodiments, the nanoparticle includes a polymeric nanoparticle, nanosphere, nanocapsule, liposome, dendrimer, polymeric micelle, or niosome. In some embodiments, the nanoparticle includes a cytotoxic agent. Antibody-nanoparticle conjugates are further described in section VIII.

Further provided herein are nucleic acid molecules that encode a polypeptide, an antibody, fusion protein, CAR, immunoconjugate, or multiple-specific antibody disclosed herein. In some embodiments, the nucleic acid molecule is operably linked to a promoter. Vectors that include the disclosed nucleic acid molecules are also provided. In some examples, the vector is an expression vector. In other examples, the vector is a viral vector. Isolated cells that include a nucleic acid molecule or vector disclosed herein are further provided. In some examples, the isolated cell is a prokaryotic cell, such as an E. coli cell. In other examples, the isolated cell is a mammalian cell, such as a human cell. Nucleic acid molecules are further described in section IX.

Compositions that include a pharmaceutically acceptable carrier and a polypeptide (for example, single-domain monoclonal antibody), fusion protein, CAR, isolated cell (such as a CAR expressing cell, for example a CAR T cell, a CAR NK cell or a CAR macrophage), immunoconjugate, ADC, multi-specific antibody, antibody-nanoparticle conjugate, isolated nucleic acid molecule or vector disclosed herein are further provided by the present disclosure. Compositions are further described in section X.

Also provided are methods of detecting PD-L1 in a sample, such as a sample obtained from a subject. In some embodiments, the method includes contacting the sample with a polypeptide (for example, antibody) disclosed herein and detecting binding of the polypeptide to the sample. Further provided are methods of diagnosing a subject as having a PD-L1-positive cancer. In some embodiments, the method includes contacting a sample obtained from the subject with a polypeptide disclosed herein and detecting binding of the polypeptide to the sample, thereby diagnosing the subject as having a PD-L1-positive cancer. In some examples of these methods, the polypeptide is directly labeled. In other examples, the method includes contacting the polypeptide with a detection antibody, and detecting the binding of the detection antibody to the polypeptide, thereby detecting the PD-L1 in the sample or diagnosing the subject as having a PD-L1-positive cancer. In some examples, the sample is obtained from a subject suspected of having a PD-L1 cancer. Diagnostic and detection methods are further described in section XII.

Further provided are methods of treating a PD-L1-positive cancer in a subject. In some embodiments, the method includes administering to the subject a therapeutically effective amount of a polypeptide (for example, single-domain monoclonal antibody), fusion protein (such as a V_{NAR}-Fc), CAR, isolated cell (such as a CAR expressing immune cell, for example a CAR T cell, a CAR NK cell or a CAR macrophage), immunoconjugate, ADC, multi-specific antibody, antibody-nanoparticle conjugate, isolated nucleic acid molecule or vector disclosed herein, thereby treating the PD-L1-positive cancer. In some examples, the PD-L1-positive cancer is a solid tumor, such as, but not limited to, a liver cancer, a breast cancer, pancreatic cancer, melanoma, non-small cell lung cancer (NSCLC), renal cell carcinoma, a bladder cancer, head and neck squamous cell carcinoma (HNSCC), a gastric cancer, urothelial carcinoma, or Merkel cell carcinoma. In specific examples, the liver cancer is HCC or the breast cancer is TNBC. Therapeutic methods are further described in section XI.

### IV. Chimeric Antigen Receptors (CARs)

The disclosed polypeptides, such as shark V_{NAR}, can also be used to produce CARs (also known as chimeric T cell receptors, artificial T cell receptors or chimeric immunoreceptors) and/or immune cells, such as T lymphocytes (such as CTLs), natural killer (NK) cells or macrophages, engineered to express CARs. Induced pluripotent stem cells (iPSCs) can also be used to express CARs. Generally, CARs include a binding moiety, an extracellular hinge and spacer element, a transmembrane region and an endodomain that performs signaling functions (Cartellieri et al., J Biomed Biotechnol 2010:956304, 2010; Dai et al., J Natl Cancer Inst 108(7):djv439, 2016). In many instances, the binding moiety is an antigen binding fragment of a monoclonal antibody, such as a scFv, or a single-domain antibody (for example, a camel or shark single-domain antibody). The spacer/hinge region typically includes sequences from IgG subclasses, such as IgG1, IgG4, IgD and CD8 domains. The transmembrane domain can be derived from a variety of different T cell proteins, such as CD3ζ, CD4, CD8 or CD28. Several different endodomains have been used to generate CARs. For example, the endodomain can consist of a signaling chain having an iTAM, such as CD3ζ or FcεRIγ. In some instances, the endodomain further includes the intracellular portion of at least one additional co-stimulatory domain, such as CD28, 4-1BB (CD137, TNFRSF9), OX-40 (CD134), ICOS, CD27 and/or DAP10.

Immune cells, such as T cells, NK cells, or macrophages, of iPSCs expressing CARs can be used to target a specific cell type, such as a PD-L1-positive tumor cell. Thus, the antibodies disclosed herein can be used to engineer immune cells or iPSCs that express a CAR containing the PD-L1-specific monoclonal antibody, thereby targeting the engineered cells to PD-L1-positive tumor cells.

Multispecific (such as bispecific) or bicistronic CARs are also contemplated by the present disclosure. In some embodiments, the multispecific or bispecific CAR includes a V_{NAR} specific for PD-L1 and a monoclonal antibody specific for a different antigen (or a different epitope of PD-L1). Similarly, a bicistronic CAR includes two CAR molecules expressed from the same construct where one CAR molecule is a PD-L1-targeted CAR and the second CAR targets a second antigen, such as GPC3 (for example using the hYP7 antibody), GPC2 or mesothelin. See, for example, Qin et al., Blood 130:810, 2017; and WO/2018/213337.

Accordingly, provided herein are CARs that include a PD-L1-specific antibody, such as any one of the V_{NAR} disclosed herein. Also provided are isolated nucleic acid molecules and vectors encoding the CARs (including bispecific and bicistronic CARs), and host cells, such as T cells, NK cells, macrophages or iPSCs expressing the CARs, bispecific CAR or bicistronic CARs. T cells, NK cells, macrophages or iPSCs expressing CARs comprised of a PD-L1-specific monoclonal antibody can be used for the treatment of a PD-L1-positive cancer. In some embodiments herein, the CAR is a bispecific CAR. In other embodiments herein, the CAR is a bicistronic CAR. In some embodiments, the bispecific or bicistronic CAR includes a monoclonal antibody (such as a single-domain antibody) or antigen-binding fragment thereof that specifically binds GPC3. In specific examples, the monoclonal antibody or antigen-binding fragment that specifically binds GPC3 comprises the CDR sequences of antibody hYP7 (see, WO 2019/094482, which is herein incorporated by reference in its entirety).

In some embodiments, the CAR includes a signal peptide sequence, for example, N-terminal to the antigen binding domain. The signal peptide sequence can be any suitable signal peptide sequence, such as a signal sequence from granulocyte-macrophage colony-stimulating factor receptor (GMCSFR), immunoglobulin light chain kappa, or IL-2. While the signal peptide sequence may facilitate expression of the CAR on the surface of the cell, the presence of the signal peptide sequence in an expressed CAR is not necessary in order for the CAR to function. Upon expression of the CAR on the cell surface, the signal peptide sequence may be cleaved off of the CAR. Accordingly, in some embodiments, the CAR lacks a signal peptide sequence.

In some embodiments, the CARs disclosed herein are expressed from a construct (such as from a lentivirus vector) that also expresses a truncated version of human EGFR (huEGFRt). The CAR and huEGFRt are separated by a self-cleaving peptide sequence (such as T2A) such that upon expression in a transduced cell, the CAR is cleaved from huEGFRt (see, e.g., WO 2019/094482, which is herein incorporated by reference).

The human epidermal growth factor receptor is comprised of four extracellular domains, a transmembrane domain and three intracellular domains. The EGFR domains are found in the following N-terminal to C-terminal order: Domain I-Domain II-Domain III-Domain IV-transmembrane (TM) domain-juxtamembrane domain-tyrosine kinase domain-C-terminal tail. Domain I and Domain III are leucine-rich domains that participate in ligand binding. Domain H and Domain IV are cysteine-rich domains and do not make contact with EGFR ligands. Domain H mediates formation of homo- or hetero-dimers with analogous domains from other EGFR family members, and Domain IV can form disulfide bonds with Domain H. The EGFR TM domain makes a single pass through the cell membrane and may play a role in protein dimerization. The intracellular domain includes the juxtamembrane domain, tyrosine kinase domain and C-terminal tail, which mediate EGFR signal transduction (Wee and Wang, Cancers 9(52), doi:10.3390/cancers9050052; Ferguson, Annu Rev Biophys 37:353-373, 2008; Wang et al., Blood 118(5):1255-1263, 2011).

A truncated version of human EGFR, referred to as “huEGFRt” includes only Domain I, Domain IV and the TM domain. Thus, huEGFRt lacks Domain I, Domain H, and all three intracellular domains. huEGFRt is not capable of binding EGF and lacks signaling activity. However, this molecule retains the capacity to bind particular EGFR-specific monoclonal antibodies, such as FDA-approved cetuximab (PCT Publication No. WO 2011/056894, which is herein incorporated by reference).

Transduction of immune cells, such as T cells, NK cells or macrophages, with a construct (such as a lentivirus vector) encoding both huEGFRt and a PD-L1-specific CAR disclosed herein allows for selection of transduced cells using labelled EGFR monoclonal antibody cetuximab (ERBITUX™). For example, cetuximab can be labeled with biotin, and transduced cells can be selected using anti-biotin magnetic beads, which are commercially available (such as from Miltenyi Biotec). Co-expression of huEGFRt also allows for in vivo tracking of adoptively transferred CAR-expressing immune cells. Furthermore, binding of cetuximab to immune cells expressing huEGFRt induces cytotoxicity of ADCC effector cells, thereby providing a mechanism to eliminate transduced immune cells in vivo (Wang et al., Blood 118(5):1255-1263, 2011), such as at the conclusion of therapy.

Also provided herein are PD-L1-specific monoclonal antibodies (such as a nanobody disclosed herein) modified to enable their use with a universal CAR system. Universal CAR systems have been developed in order to increase CAR flexibility and expand their use to additional antigens. Currently, for each patient who receives CAR immune cell therapy, autologous immune cells (such as T cells) must be cultured, expanded, and modified to express an antigen-specific CAR. This process is lengthy and expensive, limiting its use. Universal CARs are based on a system in which the signaling components of the CAR are split from the antigen-binding portion of the molecule, but come together using a “lock-key” system. For example, biotin-binding immune receptor (BBIR) CARs are comprised of an intracellular T cell signaling domain fused to an extracellular domain comprising avidin. Biotinylated antigen-specific (such as PD-L1-specific) monoclonal antibodies can then bind the BBIR to direct immune cells to antigen-expressing cells. Another example is the split, universal and programmable (SUPRA) CAR system. In the SUPRA system, the CAR includes the intracellular signaling domains fused to an extracellular leucine zipper, which is paired with an antigen-specific monoclonal antibody fused to a cognate leucine zipper. For a review of universal CAR systems, see, for example, Zhao et al., J Hematol Oncol 11(1):132, 2018; and Cho et al., Cell 173:1426-1438, 2018. In some embodiments herein, the PD-L1-specific monoclonal antibody is fused to one component of a specific binding pair. In some examples, the monoclonal antibody is fused to a leucine zipper or biotin.

Another type of universal CAR can be generated using a sortase enzyme. A sortase is a prokaryotic enzyme that modifies surface proteins by recognizing and cleaving a carboxyl-terminal sorting signal. Sortase catalyzes transpeptidation between a sortase recognition motif and a sortase acceptor motif. Thus, antigen-specific CARs can be generated by contacting an antigen-specific antibody fused to a sortase recognition motif with a portion of a CAR molecule that includes the intracellular signaling domain(s), a transmembrane region and an extracellular portion comprising a sortase acceptor motif. In the presence of the sortase enzyme, the two components become covalently attached to form a complete antigen-specific CAR. Accordingly, in some embodiments herein, a PD-L1-specific monoclonal antibody is modified to include a sortase recognition motif (see, for example, PCT Publication No. WO 2016/014553).

In some embodiments, the PD-L1 CAR is expressed in allogeneic immune cells, such as allogeneic T cells, NK cells or macrophages from a healthy donor(s). In some examples, the allogeneic immune cells are genetically engineered to express the PD-L1-targeted CAR, for example by disrupting expression of the endogenous T cell receptor by insertion of the CAR (see, for example, MacLeod et al., Mol Ther 25(4): 949-961, 2017). Gene editing can be performed using any appropriate gene editing system, such as CRISPR/Cas9, zinc finger nucleases or transcription activator-like effector nucleases (TALEN).

### V. Immunoconjugates

The disclosed single-domain monoclonal antibodies can be conjugated to a therapeutic agent or effector molecule. Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill will appreciate that therapeutic agents can include various drugs, such as vinblastine, daunomycin and the like, cytotoxins such as native or modified Pseudomonas exotoxin or diphtheria toxin, encapsulating agents (such as liposomes) that contain pharmacological compositions, radioactive agents such as ¹²⁵I, ³²P, ¹⁴C, ³H and ³⁵S, photon absorbers such as IR700, and other labels, target moieties and ligands.

The choice of a particular therapeutic agent depends on the particular target molecule or cell, and the desired biological effect. Thus, for example, the therapeutic agent can be a cytotoxin that is used to bring about the death of a particular target cell (such as a PD-L1-expressing cell). Conversely, where it is desired to invoke a non-lethal biological response, the therapeutic agent can be conjugated to a non-lethal pharmacological agent or a liposome containing a non-lethal pharmacological agent.

With the therapeutic agents and antibodies described herein, one of skill can readily construct a variety of clones containing functionally equivalent nucleic acids, such as nucleic acids which differ in sequence but which encode the same effector moiety or antibody sequence. Thus, the present disclosure provides nucleic acids encoding antibodies and conjugates and fusion proteins thereof.

Effector molecules can be linked to an antibody of interest using any number of known means. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule. Suitable linkers include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, labels (such as enzymes or fluorescent molecules), drugs, toxins, and other agents to antibodies, a skilled person will be able to determine a suitable method for attaching a given agent to an antibody or other polypeptide.

The antibodies disclosed herein can be derivatized or linked to another molecule (such as another peptide or protein). In general, the antibodies or portion thereof is derivatized such that the binding to the target antigen is not affected adversely by the derivatization or labeling. For example, the antibody can be functionally linked (by chemical coupling, genetic fusion, noncovalent association or otherwise) to one or more other molecular entities, such as another antibody (for example, a bispecific antibody or a diabody), a detection agent, a photon absorber, a pharmaceutical agent, and/or a protein or peptide that can mediate association of the antibody or antibody portion with another molecule (such as a streptavidin core region or a polyhistidine tag).

One type of derivatized antibody is produced by cross-linking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

The antibody can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging (NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-naphthalenesulfonyl chloride, phycoerythrin, lanthanide phosphors and the like. Bioluminescent markers are also of use, such as luciferase, green fluorescent protein (GFP) and yellow fluorescent protein (YFP). An antibody can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

An antibody may be labeled with a magnetic agent, such as gadolinium. Antibodies can also be labeled with lanthanides (such as europium and dysprosium), and manganese. Paramagnetic particles, such as superparamagnetic iron oxide, are also of use as labels. An antibody may also be labeled with a predetermined polypeptide epitope recognized by a secondary reporter (such as leucine zipper pair sequences, binding sites for secondary antibodies, metal binding domains, epitope tags). In some embodiments, labels are attached by spacer arms of various lengths to reduce potential steric hindrance.

An antibody can also be labeled with a radiolabeled amino acid. The radiolabel may be used for both diagnostic and therapeutic purposes. For instance, the radiolabel may be used to detect expression of a target antigen by x-ray, emission spectra, or other diagnostic techniques. Examples of labels for polypeptides include, but are not limited to, the following radioisotopes or radionucleotides: ³H, ¹⁴C, ¹⁵N, ³⁵s, ⁹⁰Y, ⁹⁹Tc, ¹¹¹In, ¹²⁵I, ¹³¹I.

An antibody disclosed herein can also be conjugated to a photon absorber. In some embodiments, the photon absorber is a phthalocyanine dye, such as, but not limited to, IRDye® 700DX (also known as “IR700”). Antibody-photoabsorber conjugates can be used for photoimmunotherapy (for example to kill PD-L1-positive tumor cells).

An antibody can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

Toxins can be employed with the monoclonal antibodies described herein to produce immunotoxins. Exemplary toxins include ricin, abrin, diphtheria toxin and subunits thereof, as well as botulinum toxins A through F. These toxins are readily available from commercial sources (for example, Sigma Chemical Company, St. Louis, MO). Contemplated toxins also include variants of the toxins described herein (see, for example, see, U.S. Pat. Nos. 5,079,163 and 4,689,401). In one embodiment, the toxin is Pseudomonas exotoxin (PE) (U.S. Pat. No. 5,602,095). As used herein “Pseudomonas exotoxin” refers to a full-length native (naturally occurring) PE or a PE that has been modified. Such modifications can include, but are not limited to, elimination of domain Ia, various amino acid deletions in domains Ib, II and III, single amino acid substitutions and the addition of one or more sequences at the carboxyl terminus (for example, see Siegall et al., J. Biol. Chem. 264:14256-14261, 1989).

PE employed with the monoclonal antibodies described herein can include the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell. Cytotoxic fragments of PE include PE40, PE38, and PE35. For additional description of PE and variants thereof, see for example, U.S. Pat. Nos. 4,892,827; 5,512,658; 5,602,095; 5,608,039; 5,821,238; and 5,854,044; U.S. Patent Application Publication No. 2015/0099707; PCT Publication Nos. WO 99/51643 and WO 2014/052064; Pai et al., Proc. Natl. Acad. Sci. USA 88:3358-3362, 1991; Kondo et al., J. Biol. Chem. 263:9470-9475, 1988; Pastan et al., Biochim. Biophys. Acta 1333:C1-C6, 1997.

Also contemplated herein are protease-resistant PE variants and PE variants with reduced immunogenicity, such as, but not limited to PE-LR, PE-6X, PE-8X, PE-LR/6X and PE-LR/8X (see, for example, Weldon et al., Blood 113(16):3792-3800, 2009; Onda et al., Proc Natl Acad Sci USA 105(32):11311-11316, 2008; and PCT Publication Nos. WO 2007/016150, WO 2009/032954 and WO 2011/032022, which are herein incorporated by reference).

In some examples, the PE is a variant that is resistant to lysosomal degradation, such as PE-LR (Weldon et al., Blood 113(16):3792-3800, 2009; PCT Publication No. WO 2009/032954). In other examples, the PE is a variant designated PE-LR/6X (PCT Publication No. WO 2011/032022). In other examples, the PE variant is PE with reducing immunogenicity. In yet other examples, the PE is a variant designated PE-LR/8M (PCT Publication No. WO 2011/032022).

Modification of PE may occur in any previously described variant, including cytotoxic fragments of PE (for example, PE38, PE-LR and PE-LR/8M). Modified PEs may include any substitution(s), such as for one or more amino acid residues within one or more T-cell epitopes and/or B cell epitopes of PE, or deletion of one or more T-cell and/or B-cell epitopes (see, for example, U.S. Patent Application Publication No. 2015/0099707).

Contemplated forms of PE also include deimmunized forms of PE, for example versions with domain II deleted (for example, PE24). Deimmunized forms of PE are described in, for example, PCT Publication Nos. WO 2005/052006, WO 2007/016150, WO 2007/014743, WO 2007/031741, WO 2009/32954, WO 2011/32022, WO 2012/154530, and WO 2012/170617.

The antibodies described herein can also be used to target any number of different diagnostic or therapeutic compounds to cells expressing PD-L1 on their surface (e.g., PD-L1-positive tumor cells). Thus, an antibody of the present disclosure can be attached directly or via a linker to a drug that is to be delivered directly to cells expressing PD-L1. This can be done for therapeutic, diagnostic or research purposes. Therapeutic agents include such compounds as nucleic acids, proteins, peptides, amino acids or derivatives, glycoproteins, radioisotopes, photon absorbers, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides.

Alternatively, the molecule linked to an antibody can be an encapsulation system, such as a nanoparticle, liposome or micelle that contains a therapeutic composition such as a drug, a nucleic acid (for example, an antisense nucleic acid), or another therapeutic moiety that is preferably shielded from direct exposure to the circulatory system. Means of preparing liposomes attached to antibodies are known (see, for example, U.S. Pat. No. 4,957,735; Connor et al., Pharm. Ther. 28:341-365, 1985).

Antibodies described herein can also be covalently or non-covalently linked to a detectable label. Detectable labels suitable for such use include any composition detectable by spectroscopic, photochemical, biochemical, immunochemical, electrical, optical or chemical means. Useful labels include magnetic beads, fluorescent dyes (for example, fluorescein isothiocyanate, Texas red, rhodamine, green fluorescent protein, and the like), radiolabels (for example, ³H, ¹²⁵I, ³⁵S, ¹⁴C, or ³²P), enzymes (such as horseradish peroxidase, alkaline phosphatase and others commonly used in an ELISA), and colorimetric labels such as colloidal gold or colored glass or plastic (such as polystyrene, polypropylene, latex, and the like) beads.

Means of detecting such labels are known. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### VI. Antibody-Drug Conjugates (ADCs)

ADCs are compounds comprised of an antigen-specific antibody (such as a single-domain antibody or antigen-binding fragment of an immunoglobulin provided herein that binds PD-L1) and a drug, for example a cytotoxic agent (such as an anti-microtubule agent or cross-linking agent). Because ADCs are capable of specifically targeting cells expressing a particular antigen, the drug can be much more potent than agents used for standard systemic therapy. For example, the most common cytotoxic drugs currently used with ADCs have an IC₅₀ that is 100- to 1000-fold more potent than conventional chemotherapeutic agents. Common cytotoxic drugs include anti-microtubule agents, such as maytansinoids and auristatins (such as auristatin E and auristatin F). Other cytotoxins for use with ADCs include pyrrolobenzodiazepines (PBDs), which covalently bind the minor groove of DNA to form interstrand crosslinks. In many instances, ADCs comprise a 1:2 to 1:4 ratio of antibody to drug (Bander, Clinical Advances in Hematology & Oncology 10(8; suppl 10):3-7, 2012).

The antibody and drug can be linked by a cleavable or non-cleavable linker. However, in some instances, it is desirable to have a linker that is stable in the circulation to prevent systemic release of the cytotoxic drug that could result in significant off-target toxicity. Non-cleavable linkers prevent release of the cytotoxic agent before the ADC is internalized by the target cell. Once in the lysosome, digestion of the antibody by lysosomal proteases results in the release of the cytotoxic agent (Bander, Clinical Advances in Hematology & Oncology 10(8; suppl 10):3-7, 2012).

One method for site-specific and stable conjugation of a drug to a monoclonal antibody (or a nanobody-Fc fusion protein) is via glycan engineering. Monoclonal antibodies have one conserved N-linked oligosaccharide chain at the Asn297 residue in the CH2 domain of each heavy chain (Qasba et al., Biotechnol Prog 24:520-526, 2008). Using a mutant β1,4-galactosyltransferase enzyme (Y289L-Gal-T1; U.S. Patent Application Publication Nos. 2007/0258986 and 2006/0084162, herein incorporated by reference), 2-keto-galactose is transferred to free GlcNAc residues on the antibody heavy chain to provide a chemical handle for conjugation.

The oligosaccharide chain attached to monoclonal antibodies can be classified into three groups based on the terminal galactose residues—fully galactosylated (two galactose residues; IgG-G2), one galactose residue (IgG-G1) or completely degalactosylated (IgG-G0). Treatment of a monoclonal antibody with β1,4-galactosidase converts the antibody to the IgG-G0 glycoform. The mutant β1,4-galactosyltransferase enzyme is capable of transferring 2-keto-galactose or 2-azido-galactose from their respective UDP derivatives to the GlcNAc residues on the IgG-G1 and IgG-G0 glycoforms. The chemical handle on the transferred sugar enables conjugation of a variety of molecules to the monoclonal antibody via the glycan residues (Qasba et al., Biotechnol Prog 24:520-526, 2008).

Provided herein are ADCs that include a drug (such as a cytotoxic agent) conjugated to a monoclonal antibody that binds (such as specifically binds) PD-L1. In some embodiments, the drug is a small molecule. In some examples, the drug is a cross-linking agent, an anti-microtubule agent and/or anti-mitotic agent, or any cytotoxic agent suitable for mediating killing of tumor cells. Exemplary cytotoxic agents include, but are not limited to, a PBD, an auristatin, a maytansinoid, dolastatin, calicheamicin, nemorubicin and its derivatives, PNU-159682, anthracycline, vinca alkaloid, taxane, trichothecene, CC1065, camptothecin, elinafide, a combretastatin, a dolastatin, a duocarmycin, an enediyne, a geldanamycin, an indolino-benzodiazepine dimer, a puromycin, a tubulysin, a hemiasterlin, a spliceostatin, or a pladienolide, as well as stereoisomers, isosteres, analogs, and derivatives thereof that have cytotoxic activity.

In some embodiments, the ADC includes a pyrrolobenzodiazepine (PBD). The natural product anthramycin (a PBD) was first reported in 1965 (Leimgruber et al., J Am Chem Soc, 87:5793-5795, 1965; Leimgruber et al., J Am Chem Soc, 87:5791-5793, 1965). Since then, a number of PBDs, both naturally-occurring and synthetic analogues, have been reported (Gerratana, Med Res Rev 32(2):254-293, 2012; and U.S. Pat. Nos. 6,884,799; 7,049,311; 7,067,511; 7,265,105; 7,511,032; 7,528,126; and 7,557,099). As one example, PBD dimers recognize and bind to specific DNA sequences, and have been shown to be useful as cytotoxic agents. PBD dimers have been conjugated to antibodies and the resulting ADC shown to have anti-cancer properties (see, for example, US 2010/0203007). Exemplary linkage sites on the PBD dimer include the five-membered pyrrolo ring, the tether between the PBD units, and the N10-C11 imine group (see WO 2009/016516; US 2009/304710; US 2010/047257; US 2009/036431; US 2011/0256157; and WO 2011/130598).

In some embodiments, the ADC includes an antibody conjugated to one or more maytansinoid molecules. Maytansinoids are derivatives of maytansine, and are mitotic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinoids are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

In some embodiments, the ADC includes an antibody conjugated to a dolastatin or auristatin, or an analog or derivative thereof (see U.S. Pat. Nos. 5,635,483; 5,780,588; 5,767,237; and 6,124,431). Auristatins are derivatives of the marine mollusk compound dolastatin-10. Dolastatins and auristatins have been shown to interfere with microtubule dynamics, GTP hydrolysis, and nuclear and cellular division (Woyke et al., Antimicrob Agents and Chemother 45(12):3580-3584, 2001) and have anticancer (U.S. Pat. No. 5,663,149) and antifungal activity (Pettit et al., Antimicrob Agents Chemother 42:2961-2965, 1998). Exemplary dolastatins and auristatins include, but are not limited to, dolastatin 10, auristatin E, auristatin F, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB), and other auristatins (see, for example, U.S. Publication No. 2013/0129753).

In some embodiments, the ADC includes an antibody conjugated to one or more calicheamicin molecules. The calicheamicin family of antibiotics, and analogues thereof, are capable of producing double-stranded DNA breaks at sub-picomolar concentrations (Hinman et al., Cancer Res 53:3336-3342, 1993; Lode et al., Cancer Res 58:2925-2928, 1998). Exemplary methods for preparing ADCs with a calicheamicin drug moiety are described in U.S. Pat. Nos. 5,712,374; 5,714,586; 5,739,116; and 5,767,285.

In some embodiments, the ADC includes an anthracycline. Anthracyclines are antibiotic compounds that exhibit cytotoxic activity. It is believed that anthracyclines can operate to kill cells by a number of different mechanisms, including intercalation of the drug molecules into the DNA of the cell thereby inhibiting DNA-dependent nucleic acid synthesis; inducing production of free radicals which then react with cellular macromolecules to cause damage to the cells; and/or interactions of the drug molecules with the cell membrane. Non-limiting exemplary anthracyclines include doxorubicin, epirubicin, idarubicin, daunomycin, daunorubicin, doxorubicin, epirubicin, nemorubicin, valrubicin and mitoxantrone, and derivatives thereof. For example, PNU-159682 is a potent metabolite (or derivative) of nemorubicin (Quintieri et al., Clin Cancer Res 11(4):1608-1617, 2005). Nemorubicin is a semisynthetic analog of doxorubicin with a 2-methoxymorpholino group on the glycoside amino of doxorubicin (Grandi et al., Cancer Treat Rev 17:133, 1990; Ripamonti et al., Br J Cancer 65:703-707, 1992).

In some embodiments, the ADC can further include a linker. In some examples, the linker is a bifunctional or multifunctional moiety that can be used to link one or more drug moieties to an antibody to form an ADC. In some embodiments, ADCs are prepared using a linker having reactive functionalities for covalently attaching to the drug and to the antibody. For example, a cysteine thiol of an antibody can form a bond with a reactive functional group of a linker or a drug-linker intermediate to make an ADC.

In some examples, a linker has a functionality that is capable of reacting with a free cysteine present on an antibody to form a covalent bond. Exemplary linkers with such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates.

In some examples, a linker has a functionality that is capable of reacting with an electrophilic group present on an antibody. Examples of such electrophilic groups include, but are not limited to, aldehyde and ketone carbonyl groups. In some cases, a heteroatom of the reactive functionality of the linker can react with an electrophilic group on an antibody and form a covalent bond to an antibody unit. Non-limiting examples include hydrazide, oxime, amino, hydrazine, thiosemicarbazone, hydrazine carboxylate and arylhydrazide.

In some examples, the linker is a cleavable linker, which facilitates release of the drug. Examples of cleavable linkers include acid-labile linkers (for example, comprising hydrazone), protease-sensitive linkers (for example, peptidase-sensitive), photolabile linkers, and disulfide-containing linkers (Chari et al., Cancer Res 52:127-131, 1992; U.S. Pat. No. 5,208,020).

The ADCs disclosed herein can be used for the treatment of a PD-L1-positive tumor alone or in combination with another therapeutic agent and/or in combination with any standard therapy for the treatment of a PD-L1-positive cancer.

### VII. Multi-Specific Antibodies

Multi-specific antibodies are recombinant proteins comprised of two or more monoclonal antibodies (such as single-domain antibodies) or antigen-binding fragments of two or more different monoclonal antibodies. For example, bispecific antibodies are comprised of two different monoclonal antibodies or antigen-binding fragments thereof. Thus, bispecific antibodies bind two different antigens and trispecific antibodies bind three different antigens.

Provided herein are multi-specific, such as trispecific or bispecific, monoclonal antibodies comprising a first PD-L1-specific monoclonal antibody. In some embodiments, the multi-specific monoclonal antibody further comprises a second antibody that specifically binds a different epitope of PD-L1 (such as atezolizumab, avelumab, durvalumab, cosibelimab, KN035 (envafolimab), BMS-936559, BMS935559, MEDI-4736, MPDL-3280A, or MEDI-4737) or a different cell-surface antigen. In some embodiments, the multi-specific monoclonal antibody further comprises a second antibody that specifically binds PD-1 (such as nivolumab, JTX-4014 by Jounce Therapeutics, nivolumab, pembrolizumab, pidilizumab, cemiplimab, spartalizumab (PDR001), camrelizumab (SHR1210), sintilimab (IBI308), tislelizumab (BGB-A317), toripalimab (JS 001, dostarlimab (TSR-042, WBP-285), INCMGA00012 (MGA012), AMP-224, or AMP-514). In some embodiments, the multi-specific monoclonal antibody further comprises a second antibody that specifically binds CTLA-4 (such as ipilimumab or tremelimumab). Also provided are isolated nucleic acid molecules and vectors encoding the multi-specific antibodies, and host cells comprising the nucleic acid molecules or vectors. Multi-specific antibodies comprising a PD-L1-specific antibody can be used for the treatment of a PD-L1-positive cancer. Thus, provided herein are methods of treating a subject with a PD-L1-positive cancer by administering to the subject a therapeutically effective amount of the PD-L1-targeting multi-specific antibody.

### VIII. Antibody-Nanoparticle Conjugates

The monoclonal antibodies disclosed herein can be conjugated to a variety of different types of nanoparticles to deliver cytotoxic agents directly to PD-L1-expressing cells via binding of the antibody to PD-L1 expressed on the surface of cells. The use of nanoparticles reduces off-target side effects and can also improve drug bioavailability and reduce the dose of a drug required to achieve a therapeutic effect. Nanoparticle formulations can be tailored to suit the drug that is to be carried or encapsulated within the nanoparticle. For example, hydrophobic molecules can be incorporated inside the core of a nanoparticle, while hydrophilic drugs can be carried within an aqueous core protected by a polymeric or lipid shell. Examples of nanoparticles include, but at not limited to, nanospheres, nanocapsules, liposomes, dendrimers, polymeric micelles, niosomes, and polymeric nanoparticles (Fay and Scott, Immunotherapy 3(3):381-394, 2011).

Liposomes are common types of nanoparticles used for drug delivery. An antibody conjugated to a liposome is often referred to as an “immunoliposome.” The liposomal component of an immunoliposome is typically a lipid vesicle of one or more concentric phospholipid bilayers. In some cases, the phospholipids are composed of a hydrophilic head group and two hydrophobic chains to enable encapsulation of both hydrophobic and hydrophilic drugs. Conventional liposomes are rapidly removed from the circulation via macrophages of the reticuloendothelial system (RES). To generate long-circulating liposomes, the composition, size and charge of the liposome can be modulated. The surface of the liposome may also be modified, such as with a glycolipid or sialic acid. For example, the inclusion of polyethylene glycol (PEG) significantly increases circulation half-life. Liposomes for use as drug delivery agents, including for preparation of immunoliposomes, have been described (see, for example, Paszko and Senge, Curr Med Chem 19(31)5239-5277, 2012; Immordino et al., Int J Nanomedicine 1(3):297-315, 2006; U.S. Patent Application Publication Nos. 2011/0268655; 2010/00329981).

Niosomes are non-ionic surfactant-based vesicles having a structure similar to liposomes. The membranes of niosomes are composed only of nonionic surfactants, such as polyglyceryl-alkyl ethers or N-palmitoylglucosamine. Niosomes range from small, unilamellar to large, multilamellar particles. These nanoparticles are monodisperse, water-soluble, chemically stable, have low toxicity, are biodegradable and non-immunogenic, and increase bioavailability of encapsulated drugs.

Dendrimers include a range of branched polymer complexes. These nanoparticles are water-soluble, biocompatible and are sufficiently non-immunogenic for human use. Generally, dendrimers consist of an initiator core, surrounded by a layer of a selected polymer that is grafted to the core, forming a branched macromolecular complex. Dendrimers are typically produced using polymers such as poly(amidoamine) or poly(L-lysine). Dendrimers have been used for a variety of therapeutic and diagnostic applications, including for the delivery of DNA, RNA, bioimaging contrast agents, chemotherapeutic agents and other drugs.

Polymeric micelles are composed of aggregates of amphiphilic co-polymers (consisting of both hydrophilic and hydrophobic monomer units) assembled into hydrophobic cores, surrounded by a corona of hydrophilic polymeric chains exposed to the aqueous environment. In many cases, the polymers used to prepare polymeric micelles are heterobifunctional copolymers composed of a hydrophilic block of PEG, poly(vinyl pyrrolidone) and hydrophobic poly(L-lactide) or poly(L-lysine) that forms the particle core. Polymeric micelles can be used to carry drugs that have poor solubility. These nanoparticles have been used to encapsulate a number of drugs, including doxorubicin and camptothecin. Cationic micelles have also been developed to carry DNA or RNA molecules.

Polymeric nanoparticles include both nanospheres and nanocapsules. Nanospheres consist of a solid matrix of polymer, while nanocapsules contain an aqueous core. The formulation selected typically depends on the solubility of the therapeutic agent to be carried/encapsulated; poorly water-soluble drugs are more readily encapsulated within nanospheres, while water-soluble and labile drugs, such as DNA and proteins, are more readily encapsulated within nanocapsules. The polymers used to produce these nanoparticles include, for example, poly(acrylamide), poly(ester), poly(alkylcyanoacrylates), poly(lactic acid) (PLA), poly(glycolic acids) (PGA), and poly(D,L-lactic-co-glycolic acid) (PLGA).

Antibodies can be conjugated to a suitable nanoparticle according to standard known methods. For example, conjugation can be either covalent or non-covalent. In some embodiments in which the nanoparticle is a liposome, the antibody is attached to a sterically stabilized, long circulation liposome via a PEG chain. Coupling of antibodies or antibody fragments to a liposome can also involve thioester bonds, for example by reaction of thiols and maleimide groups. Cross-linking agents can be used to create sulfhydryl groups for attachment of antibodies to nanoparticles (Paszko and Senge, Curr Med Chem 19(31)5239-5277, 2012).

### IX. Nucleic Acid Molecules

Nucleic acid molecules (for example, DNA, cDNA, mRNA, or RNA molecules) encoding the amino acid sequences of the disclosed polypeptides, antibodies, fusion proteins, and conjugates that specifically bind to PD-L1, are provided. Nucleic acid molecules encoding these molecules can readily be produced using the amino acid sequences provided herein (such as the CDR sequences and the variable domain sequences), sequences available (such as framework or constant region sequences), and the genetic code. In some embodiments, the nucleic acid molecules can be expressed in a host cell (such as a mammalian cell or a bacterial cell) to produce a disclosed polypeptide, antibody, fusion protein or antibody conjugate (e.g., CAR, immunotoxin, multi-specific antibody).

In some embodiments, the nucleotide sequence of the nucleic acid molecule encoding a polypeptide (such as a V_{NAR} single-domain antibody) disclosed herein is at least 80%, at least 85%, at least 90%, at least 95%, at least 96%, at least 97%, at least 98%, or at least 99% identical to any one of SEQ ID NOs: 17-28. In some examples, the nucleotide sequence of the nucleic acid molecule encoding a disclosed polypeptide comprises or consists of any one of SEQ ID NOs: 17-28.

Shark V_{NAR} B2
(SEQ ID NO: 17)
GCTCGAGTGGACCAAACACCGAGATCAGTAACAAAGGAGACGGGCGAATCACTGACCATCAA
CTGTGTCCTACGAGATAGTAGCTATGCATTGGGCAGCACGTACTGGTATCGAAAAAAATCGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GTACACGTCGCGCCTCCGGCGGGAGGGGCCCTTGTCGTGGGACGGGAACACGGTGTACGGAGG
TGGCACTGTCGTGACTGTGAAT
Shark V_{NAR} F5
(SEQ ID NO: 18)
GCTCGAGTGGACCAAACACCGAGATCAGTAACAAAGGAGACGGGCGAATCACTGACCATCAA
CTGTGTCCTACGAGATACTAACTATGCATTGGGCAGCACGTACTGGTATCGAAAAAAATTGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GAACCTCGGGCACCCGATGTACATCGCGGGCGCCATCTGCCGGCCCTTGCCGATCTACGGAGG
TGGCACTGTCGTGACTGTGAAT
Shark V_{NAR} A11
(SEQ ID NO: 19)
GCTCGAGTGGACCAAACACCGAGATCAGTAACAAAGGAGACGGGCGAATCACTGACCATCAA
CTGTGTCCTACGAGATACTAGCTATGCATTGGGCAGCACGTACTGGTATCGAAAAAAATCGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GTTCAAGAGCAACACCGAGTTCCTCAACCCCTTGACGGTCTCGATGAGCACGATCTACGGAGG
TGGCACTGTCGTGACTGTGAAT
Shark V_{NAR} A3
(SEQ ID NO: 20)
GCTCGAGTGGACCAAACACCGCAAACAATAACAAAGGAGACGGGCGAATCACTGACCATCAA
CTGTGTCCTACGAGATAGTAACTATGCATTGGGCAGCACGTACTGGTATCGAAAAAAATCGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GACGCAGAAGGCCATCCCCTGCAACATGAACGGGTACGTGTGCGGGGTGACGATCTACGGAGG
TGGCACTGTCGTGACTGTGAAT
Shark V_{NAR} A9
(SEQ ID NO: 21)
GCTCGAGTGGACCAAACACCGCAAACAATAACAAAGGAGACGGGCGAATCACTGACCATCAA
CTGTGTCCTACGAGATACTAACTATGCATTGGGCAGCACGTACTGGTATCGAAAAAAATTGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GAACACGTCCCCGTGGCTGACGTACAGCCCCTGGACCGTCAGCGGGCAGACGTCGTACGGAGG
TGGCACTGTCGTGACTGTGAAT
Shark V_{NAR} A2
(SEQ ID NO: 22)
GCTCGAGTGGACCAAACACCGAGATCAGTAACAAAGGAGACGGGCGAATCACTGACCATCAA
CTGTGTCCTACGAGATACTAGCTATGCATTGGGCAGCACGTACTGGTATCGAAAAAAATTGGG
CTCAACAAACGAGGAGAGCATATCGAAAGGTGGACGATATGTTGAAACAGTTAACAGCGGCTC
AAAGACCTTTTCTTTGAGAATTAATGATCTAACAGTTGAAGACAGTGGCACGTATCGATGCAA
GGTGCAGACCACCTGGTCCTACTTGAGCCCGTACAAGATCGAGTTCGCGACCGTCTACGGAGG
TGGCACTGTCGTGACTGTGAAT
Shark V_{NAR} A10
(SEQ ID NO: 23)
GCTCGAGTGGACCAAACACCGAGATCAGTAACAAAGGAGACGGGCGAATCACTGACCATCAA
CTGTGTCCTACGAGATACTAACTATGCATTGGGCAGCACGTACTGGTATCGAAAAAAATTGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GTCGACCACGCCCTGGAACAACCTCACGTCGTTCACGTCCGAGCGGGTGACCATCTACGGAGG
TGGCACTGTCGTGACTGTGAAT
Shark V_{NAR} A7
(SEQ ID NO: 24)
GCTCGAGTGGACCAAACACCGAGATCAGTAACAAAGGAGACGGGCGAATCACTGACCATCAA
CTGTGTCCTACGAGATACTAGCTATGCATTGGGCAGCACGTACTGGTATCGAAAAAAATTGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GATGAACCAGCGCTACCCCTGCGACAACAACTCCTGGTGGTCCCTGTACTGCGGGACCACGGT
CTACGGAGGTGGCACTGTCGTGACTGTGAAT
Shark V_{NAR} A6
(SEQ ID NO: 25)
GCTCGAGTGGACCAAACACCGCAAACAATAACAAAGGAGACGGGCGAATCACTGACCATCAA
CTGTGTCCTACGAGATACTAGCTATGCATTGGGCAGCACGTACTGGTATCGAAAAAAATCGGG
CTCAACAAACGAGGAGAGCATATCGAAAGGTGGACGATATGTTGAAACAGTTAACAGCGGAT
CAAAGTCCTTTTCTTTGAGAATTAATGATCTACGAGTTGAAGACAGTGGCACGTATCGATGCAA
GTACTCGAGCTCCTGGCAGAAGGCCACCGAGGGCGTCTGGGAGGCGATGACGGAGTACGGAG
GTGGCACTGTCGTGACTGTGAAT
Shark V_{NAR} C4
(SEQ ID NO: 26)
GCTCGAGTGGACCAAACACCGAGATCAGTAACAAAGGAGACGGGCGAATCACTGACCATCAA
CTGTGTCCTACGAGATACTAACTATGCATTGGGCAGCACGTACTGGTATCGAAAAAAATCGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GCAGTCGGCCTCGCTCTGGATCCCGAACCTCCTCCGCTGGGTGCCCGTGATCTCGTACGGAGGT
GGCACTGTCGTGACTGTGAAT
Shark V_{NAR} A1
(SEQ ID NO: 27)
GCTCGAGTGGACCAAACACCGAGATCAGTAACAAAGGAGACGGGCGAATCACTGACCATCAA
CTGTGTTCTACGAGATACTAACTATGCATTGGGCAGCACGTACTGGTATCGAAAAAAATCGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GACGTCGTCGAAGCCCCTCCTCGTCTCCCACAACGTGTGGTCGGCCTGGACGGAGTACGGAGG
TGGCACTGTCGTGACTGTGAAT
Shark V_{NAR} D12
(SEQ ID NO: 28)
GCTCGAGTGGACCAAACACCGAGATCAGTAACAAAGGAGACGGGCGAATCACTGACCATCAA
CTGTGTCCTACGAGATACTAACTATGCATTGGGCAGCACGTACTGGTATCGAAAAAAATCGGG
CTCAACAAACGAGGAGAGCATATCGAAAGGTGGACGATATGTTGAAACAGTTAACAGCGGAT
CAAAGTCCTTTTCTTTGAGAATTAATGATCTAACAGTTGAAGACAGTGGCACGTATCGATGCAA
GCACCAGTCGTCCTGGAGGCGCCAGGCCCCGCGCGTGATGGAGATGCAGACGTTGTACGGAGG
TGGCACTGTCGTGACTGTGAAT

The genetic code can be used to construct a variety of functionally equivalent nucleic acid sequences, such as nucleic acids that differ in their sequence but which encode the same antibody sequence, or encode a conjugate or fusion protein including the nanobody sequence.

Nucleic acid molecules encoding the polypeptides, antibodies, fusion proteins, and conjugates that specifically bind to PD-L1 can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as in bacterial, plant, yeast, insect and mammalian cells. The antibodies and conjugates can be expressed as individual proteins including the single-domain antibody (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011).

One or more DNA sequences encoding the polypeptides, antibodies, fusion proteins, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells, for example mammalian cells, such as the COS, CHO, HeLa and myeloma cell lines, can be used to express the disclosed antibodies and antigen binding fragments. Methods of stable transfer, meaning that the foreign DNA is continuously maintained in the host, may be used.

The expression of nucleic acids encoding the antibodies and conjugates described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding an antibody described herein without diminishing its biological activity. Some modifications can be made to facilitate the cloning, expression, or incorporation of the antibody into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the polypeptides, antibodies, fusion proteins, and conjugates can be purified according to standard procedures, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The polypeptides, antibodies, fusion proteins, and conjugates need not be 100% pure. Once purified, partially or to homogeneity as desired, if to be used prophylactically, the antibodies should be substantially free of endotoxin.

Methods for expression of polypeptides, antibodies, fusion proteins, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### X. Compositions and Administration

Compositions are provided that include one or more of the disclosed polypeptides (such as monoclonal antibodies) that bind (for example specifically bind) PD-L1 in a carrier. Compositions comprising fusion proteins (such as nanobody-Fc fusion proteins), ADCs, CARs (and immune cells expressing CARs), multi-specific (such as bispecific or trispecific) antibodies, antibody-nanoparticle conjugates, immunoliposomes and immunoconjugates are also provided, as are nucleic acid molecule and vectors encoding the antibodies or antibody conjugates. The compositions can be prepared in unit dosage form for administration to a subject. The amount and timing of administration are at the discretion of the treating clinician to achieve the desired outcome. The polypeptide, antibody, fusion protein, ADC, CAR, CAR-expressing cell, multi-specific antibody, antibody-nanoparticle conjugate, immunoliposome or immunoconjugate can be formulated for systemic or local administration.

The compositions for administration can include a solution of the polypeptide, antibody, fusion protein, ADC, CAR, CAR-expressing cell (such as a T cell, NK cell, macrophage or iPSC), multi-specific (such as bispecific or trispecific) antibody, antibody-nanoparticle conjugate, immunoliposome or immunoconjugate in a pharmaceutically acceptable carrier, such as an aqueous carrier. A variety of aqueous carriers can be used, for example, buffered saline and the like. These solutions are sterile and generally free of undesirable matter. These compositions may be sterilized by conventional sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical pharmaceutical composition for intravenous administration includes about 0.1 to 10 mg of polypeptide, such as an antibody (or fusion protein, ADC, CAR, multi-specific antibody, antibody-nanoparticle conjugate, or immunoconjugate), per subject per day. Dosages from 0.1 up to about 100 mg per subject per day may be used, particularly if the agent is administered to a secluded site and not into the circulatory or lymph system, such as into a body cavity or into a lumen of an organ. In some embodiments, the composition can be a liquid formulation including one or more antibodies in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml. Actual methods for preparing administrable compositions will be known or apparent to a skilled person and are described in more detail in such publications as Remington: The Science and Practice of Pharmacy, The University of the Sciences in Philadelphia, Editor, Lippincott, Williams, & Wilkins, Philadelphia, PA, 21^{st} Edition (2005).

The polypeptides and monoclonal antibodies disclosed herein can also be administered by other routes, including via inhalation or oral.

Polypeptides and antibodies (or antibody conjugates, or nucleic acid molecules encoding such molecules) may be provided in lyophilized form and rehydrated with sterile water before administration, although they are also provided in sterile solutions of known concentration. The antibody solution can be added to an infusion bag containing 0.9% sodium chloride, USP, and in some cases administered at a dosage of from 0.5 to 15 mg/kg of body weight. Considerable experience is available in the administration of antibody drugs, which have been marketed in the U.S. since the approval of RITUXAN™ in 1997. Polypeptides, antibodies, Fc fusion proteins, ADCs, CARs (or CAR-expressing cells), multi-specific (such as bispecific or trispecific) antibodies, antibody-nanoparticle conjugates, immunoliposomes or immunoconjugates can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include, for example, microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the polypeptide/antibody-based compositions disclosed herein. Various degradable and nondegradable polymeric matrices for use in controlled drug delivery are known (Langer, Accounts Chem. Res. 26:537-542, 1993). For example, the block copolymer, poloxamer 407, exists as a viscous yet mobile liquid at low temperatures but forms a semisolid gel at body temperature. It is an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### XI. Therapeutic Methods

The antibodies, compositions, CARs (and CAR-expressing immune cells or iPSCs), ADCs, multi-specific (such as bispecific or trispecific) antibodies, antibody-nanoparticle conjugates, immunoliposomes and immunoconjugates disclosed herein can be administered to slow or inhibit the growth of tumor cells or inhibit the metastasis of tumor cells, such as a PD-L1-positive solid tumor. In these applications, a therapeutically effective amount of a composition is administered to a subject in an amount sufficient to inhibit growth, replication or metastasis of cancer cells, or to inhibit a sign or a symptom of the cancer. Suitable subjects may include those diagnosed with a solid tumor that expresses PD-L1, such as, but not limited to, liver cancer, breast cancer, pancreatic cancer, melanoma, non-small cell lung cancer (NSCLC), renal cell carcinoma, bladder cancer, head and neck squamous cell carcinoma (HNSCC), gastric cancer, urothelial carcinoma, and Merkel cell carcinoma.

Provided herein is a method of treating a PD-L1-positive cancer in a subject by administering to the subject a therapeutically effective amount of a PD-L1-specific polypeptide (such as a single-domain antibody), immunoconjugate, CAR (or an immune cell expressing a CAR), ADC, multi-specific (such as bispecific or trispecific) antibody, antibody-nanoparticle conjugate, immunoliposome or composition disclosed herein. Also provided herein is a method of inhibiting tumor growth or metastasis of a PD-L1-positive cancer in a subject by administering to the subject a therapeutically effective amount of a PD-L1-specific polypeptide (such as a single-domain antibody), immunoconjugate, CAR (such as an immune cell expressing a CAR), ADC, multi-specific (such as bispecific or trispecific) antibody, antibody-nanoparticle conjugate, immunoliposome or composition disclosed herein. In some embodiments, the PD-L1-positive cancer is a solid tumor, such as liver cancer (e.g., HCC), breast cancer (e.g., TNBC), pancreatic cancer, melanoma, NSCLC, renal cell carcinoma, bladder cancer, HNSCC, gastric cancer, urothelial carcinoma, or Merkel cell carcinoma.

The tumor does not need to be completely eliminated or inhibited for the method to be effective. For example, the method can decrease tumor size (e.g., volume) or metastasis by a particular amount, for example by at least 10%, at least 20%, at least 30%, at least 40%, at least 50%, at least 60%, at least 70%, at least 80%, at least 90%, at least 95%, at least 98%, at least 99%, or even 100% as compared to the absence of the treatment.

A therapeutically effective amount of a PD-L1-specific polypeptide, monoclonal antibody, ADC, CAR (for example an immune cell or iPSC expressing a CAR), multi-specific (such as bispecific or trispecific) antibody, immunoconjugate, immunoliposome or composition disclosed herein will depend upon the severity of the disease, the type of disease, and the general state of the patient's health. A therapeutically effective amount of the antibody-based composition is that which provides either subjective relief of a symptom(s) or an objectively identifiable improvement as noted by the clinician or other qualified observer. Polypeptides, such as antibodies and conjugates thereof, can be administered, for example, by intravenous infusion. Doses of the antibody or conjugate thereof can vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some embodiments, the dose of the antibody or conjugate can be from about 0.5 mg/kg to about 5 mg/kg, such as a dose of about 1 mg/kg, about 2 mg/kg, about 3 mg/kg, about 4 mg/kg or about 5 mg/kg. The antibody or conjugate is administered according to a dosing schedule determined by a medical practitioner. In some examples, the antibody or conjugate is administered weekly, every two weeks, every three weeks or every four weeks.

In some embodiments, a subject is administered DNA or RNA encoding a disclosed antibody to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, or antigen binding fragments thereof, can be placed under the control of a promoter to increase expression. The methods include liposomal delivery of the nucleic acids. Such methods can be applied to the production of an antibody, or antigen binding fragments thereof.

In several embodiments, a subject (such as a human subject with a PD-L1-positive tumor) is administered an effective amount of a viral vector that includes one or more nucleic acid molecules encoding a disclosed antibody. The viral vector is designed for expression of the nucleic acid molecules encoding a disclosed polypeptide (e.g., antibody), and administration of the effective amount of the viral vector to the subject leads to expression of an effective amount of the antibody in the subject. Non-limiting examples of viral vectors that can be used to express a disclosed antibody or antigen binding fragment in a subject include those provided in Johnson et al., Nat. Med., 15(8):901-906, 2009 and Gardner et al., Nature, 519(7541):87-91, 2015, each of which is incorporated by reference herein in its entirety.

In one embodiment, a nucleic acid encoding a disclosed polypeptide, antibody, or conjugate thereof, is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including a disclosed polypeptide, antibody or antibody conjugate, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit growth or metastasis of a PD-L1-positive cancer without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized.

The PD-L1-specific polypeptide, antibody, antibody conjugate, or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In some embodiments, the composition is administered by inhalation, such as by using an inhaler. In one embodiment, the polypeptide, antibody, antigen binding fragment, or nucleic acid molecule encoding such molecules, or a composition including such molecules, is administered by a single subcutaneous, intravenous, intra-arterial, intraperitoneal, intramuscular, intradermal or intrathecal injection once a day. The polypeptide, antibody, antigen binding fragment, bispecific antibody, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the polypeptide, antibody, antibody conjugate, or nucleic acid molecule encoding such molecules, or a composition including such molecules, over a pre-determined period. The osmotic pump or mini-pump can be implanted subcutaneously, or near a target site.

In one example, a PD-L1-specific polypeptide provided herein is conjugated to IR700, and photoimmunotherapy is used to treat a PD-L1-positive cancer. For example, such a method can include administering to the subject with a PD-L1-positive cancer a therapeutically effective amount of one or more PD-L1-specific antibody-IR700 conjugates, wherein the PD-L1-specific antibody specifically binds to PD-L1-expressing cells. Following administration of the conjugate, irradiation is performed at a wavelength of 660 to 740 nm (such as 660 to 710 nm, for example, 680 nm) and at a dose of at least 1 J cm⁻², thereby treating the PD-L1-positive cancer in the subject. In some examples, the PD-L1-positive tumor is irradiated at a wavelength of 660 to 740 nm (such as 660 to 710 nm, for example, 680 nm) at a dose of at least 1 J cm⁻² (such as at least 1 J cm⁻², at least 4 J cm⁻², at least 10 J cm⁻², at least 50 J cm⁻², or at least 100 J cm⁻²) thereby treating the cancer in the subject. In some examples, multiple rounds of treatment are performed, such as 2, 3, 4, 5, 6, 7, 8, 9 or 10 treatment cycles. In particular examples, a therapeutically effective dose of a PD-L1-specific antibody-IR700 conjugates is at least 0.5 milligram per 60 kilogram (mg/kg), at least 5 mg/60 kg, at least 10 mg/60 kg, at least 20 mg/60 kg, at least 30 mg/60 kg, at least 50 mg/60 kg, for example 0.5 to 50 mg/60 kg, such as a dose of 1 mg/60 kg, 2 mg/60 kg, 5 mg/60 kg, 20 mg/60 kg, or 50 mg/60 kg, for example when administered intravenously. In another example, a therapeutically effective dose of a PD-L1-specific antibody-IR700 conjugates is at least 10 μg/kg, such as at least 100 μg/kg, at least 500 μg/kg, or at least 500 μg/kg, for example 10 μg/kg to 1000 μg/kg, such as a dose of 100 μg/kg, 250 μg/kg, about 500 μg/kg, 750 μg/kg, or 1000 μg/kg, for example when administered i.p. In one example, a therapeutically effective dose of an PD-L1-specific antibody-IR700 conjugates is at least 1 μg/ml, such as at least 500 μg/ml, such as between 20 μg/ml to 100 μg/ml, such as 10 μg/ml, 20 μg/ml, 30 μg/ml, 40 μg/ml, 50 μg/ml, 60 μg/ml, 70 μg/ml, 80 μg/ml, 90 μg/ml or 100 μg/ml administered in a topical solution.

In some embodiments, the treatment methods further include administration of other anti-cancer agents or therapeutic treatments. Any suitable anti-cancer agent can be administered in combination with the compositions disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and other antibodies that specifically target cancer cells.

Non-limiting examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

Non-limiting examples of antimetabolites include folic acid analogs (such as methotrexate), pyrimidine analogs (such as 5-FU or cytarabine), and purine analogs, such as mercaptopurine or thioguanine.

Non-limiting examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitomycin C), and enzymes (such as L-asparaginase).

Non-limiting examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocortical suppressants (such as mitotane and aminoglutethimide).

Non-limiting examples of hormones and antagonists include adrenocorticosteroids (such as prednisone), progestins (such as hydroxyprogesterone caproate, medroxyprogesterone acetate, and megestrol acetate), estrogens (such as diethylstilbestrol and ethinyl estradiol), antiestrogens (such as tamoxifen), and androgens (such as testosterone propionate and fluoxymesterone). Examples of the most commonly used chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

Exemplary biologics that can be used in combination with the disclosed methods include one or more monoclonal antibodies (mAbs) used to treat cancer, such as mAbs specific for EGFR (e.g., cetuximab), VEGF (e.g., bevacizumab), PD-1 (e.g., nivolumab, JTX-4014 by Jounce Therapeutics, nivolumab, pembrolizumab, pidilizumab, cemiplimab, spartalizumab (PDR001), camrelizumab (SHR1210), sintilimab (IBI308), tislelizumab (BGB-A317), toripalimab (JS 001, dostarlimab (TSR-042, WBP-285), INCMGA00012 (MGA012), AMP-224, or AMP-514), PD-L1 (e.g., atezolizumab, avelumab, durvalumab, cosibelimab, KN035 (envafolimab), BMS-936559, BMS935559, MEDI-4736, MPDL-3280A, or MEDI-4737), CD25 (e.g., daclizumab or basiliximab), CD20 (e.g., Tositumomab (Bexxar®); Rituximab (Rituxan, Mabthera); Ibritumomab tiuxetan (Zevalin, for example in combination with yttrium-90 or indium-111 therapy); Ofatumumab (Arzerra®), veltuzumab, obinutuzumab, ublituximab, ocaratuzumab), CD22 (e.g., narnatumab, inotuzumab ozogamicin, moxetumomab pasudotox) or CTLA4 (e.g., ipilimumab, tremelimumab). In some examples, the additional therapeutic agent administered is an anti-cancer monoclonal antibody, for example one or more of: 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof. In a specific example, the disclosed methods are used in combination with a therapeutic PD-1 mAb, such as one or more of nivolumab, JTX-4014 by Jounce Therapeutics, nivolumab, pembrolizumab, pidilizumab, cemiplimab, spartalizumab (PDR001), camrelizumab (SHR1210), sintilimab (IBI308), tislelizumab (BGB-A317), toripalimab (JS 001, dostarlimab (TSR-042, WBP-285), INCMGA00012 (MGA012), AMP-224, and AMP-514),

In some examples, the methods further include surgical treatment, for example surgical resection of the cancer or a portion of it. In some examples, the methods further include administration of radiotherapy, for example administration of radioactive material or energy (such as external beam therapy) to the tumor site to help eradicate the tumor or shrink it prior to surgical resection.

### XII. Methods for Diagnosis and Detection

Methods are also provided for the detection of the presence of PD-L1 in vitro or in vivo. For example, the disclosed polypeptides, such as nanobodies, can be used for in vivo imaging to detect a PD-L1-positive cancer. To use the disclosed polypeptides (such as antibodies or nanobodies) as diagnostic reagents in vivo, the polypeptides are labelled with a detectable moiety, such as a radioisotope, fluorescent label, or positron emitting radionuclides. As one example, the nanobodies disclosed herein can be conjugated to a positron emitting radionuclide for use in positron emission tomography (PET); this diagnostic process is often referred to as immunoPET. While full length antibodies can make good immunoPET agents, their biological half-life necessitates waiting several days prior to imaging, which increases associated non-target radiation doses. Smaller, single domain antibodies/nanobodies, such as those disclosed herein, have biological half-lives amenable to same day imaging.

To use the disclosed polypeptides (such as antibodies or nanobodies) as diagnostic reagents in vitro, the polypeptides can be directly or indirectly labelled with a detectable moiety (e.g., by using a labeled secondary antibody that binds to the PD-L1 antibody), such as a radioisotope, enzyme, or fluorescent label. In some examples, the presence of PD-L1 is detected in a biological sample from a subject and can be used to identify a subject with a PD-L1-positive cancer. The sample can be any sample, including, but not limited to, blood, serum, urine, semen, sputum, saliva, mucus, nasal wash, nasopharyngeal samples, oropharyngeal samples, tissue, cells, tissue biopsy, fine needle aspirate, surgical specimen, feces, cerebral spinal fluid (CSF), and bronchoalveolar lavage (BAL) fluid. Biological samples also include sections of tissues, for example, frozen sections taken for histological purposes. The method of detection can include contacting a cell or sample, with an antibody or antibody conjugate (e.g., a conjugate including a detectable marker) that specifically binds to PD-L1 under conditions sufficient to form an immune complex, and detecting the immune complex (e.g., by detecting a detectable marker conjugated to the antibody or antigen binding fragment).

Provided herein is a method of determining if a subject has a PD-L1-positive cancer by contacting a sample from the subject with a PD-L1-specific polypeptide (such as a single-domain antibody) disclosed herein; and detecting binding of the polypeptide to the sample. An increase in binding of the polypeptide to the sample as compared to binding of the polypeptide to a control sample identifies the subject as having a PD-L1-positive cancer.

In another embodiment, provided is a method of confirming a diagnosis of a PD-L1-positive cancer in a subject by contacting a sample from a subject diagnosed with a PD-L1-positive cancer with a PD-L1-specific polypeptide (such as a single-domain antibody) disclosed herein; and detecting binding of the polypeptide to the sample. An increase in binding of the polypeptide to the sample as compared to binding of the polypeptide to a control sample confirms the diagnosis of a PD-L1-positive cancer in the subject.

In one embodiment, the polypeptide, antibody or antigen binding fragment is directly labeled with a detectable marker. In another embodiment, the polypeptide/antibody that binds PD-L1 (the primary antibody) is unlabeled and a secondary antibody or other molecule that can bind the primary antibody is utilized for detection. The secondary antibody that is chosen is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

In an alternative embodiment, PD-L1 can be assayed in a biological sample by a competition immunoassay utilizing PD-L1 standards labeled with a detectable substance and an unlabeled antibody that specifically binds PD-L1. In this assay, the biological sample, the labeled PD-L1 standards and the antibody that specifically binds PD-L1 are combined and the amount of labeled PD-L1 standard bound to the unlabeled antibody is determined. The amount of PD-L1 in the biological sample is inversely proportional to the amount of labeled PD-L1 standard bound to the antibody that specifically binds PD-L1.

The immunoassays and methods disclosed herein can be used for a number of purposes. In one embodiment, the antibody that specifically binds PD-L1 may be used to detect the production of PD-L1 in cells in cell culture. In another embodiment, the antibody can be used to detect the amount of PD-L1 in a biological sample, such as a sample obtained from a subject having or suspected or having a PD-L1-positive cancer.

In one embodiment, a kit is provided for detecting PD-L1 in a biological sample, such as a tissue biopsy, fine needle aspirate, core biopsy, blood, serum, urine, semen, CSF, nasopharyngeal, oropharyngeal, sputum, or saliva sample. Kits for detecting PD-L1-positive cells can include a polypeptide (such as an antibody) that specifically binds PD-L1, such as any of the nanobodies disclosed herein. In a further embodiment, the polypeptide is labeled (for example, with a fluorescent, radioactive, or an enzymatic label). In some examples, the polypeptide/antibody is present on a solid support, such as a bead or multi-well plate. In some examples, the kit further includes a detectably labeled secondary antibody that permits detection of the antibody that specifically binds PD-L1.

In one embodiment, a kit includes instructional materials disclosing means of use of an antibody that binds PD-L1. The instructional materials may be written, in an electronic form or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. The kits may additionally include materials to obtain a sample, such as a swab, syringe, needle, and the like. Such kits and appropriate contents are well known.

In one embodiment, the diagnostic kit comprises an immunoassay. Although the details of the immunoassays may vary with the particular format employed, the method of detecting PD-L1 in a biological sample generally includes the steps of contacting the biological sample with an antibody which specifically reacts, under immunologically reactive conditions, to PD-L1. The antibody is allowed to specifically bind under immunologically reactive conditions to form an immune complex, and the presence of the immune complex (bound antibody) is detected directly or indirectly.

The polypeptide (such as V_{NAR} antibodies) disclosed herein can also be utilized in immunoassays, such as, but not limited to radioimmunoassays (RIAs), ELISA, lateral flow assay (LFA), or immunohistochemical assays. The polypeptides can also be used for fluorescence activated cell sorting (FACS), such as for identifying/detecting PD-L1-positive cells. FACS employs a plurality of color channels, low angle and obtuse light-scattering detection channels, and impedance channels, among other more sophisticated levels of detection, to separate or sort cells (see U.S. Pat. No. 5,061,620). Any of the polypeptides (such as single-domain antibodies) that bind PD-L1, as disclosed herein, can be used in these assays. Thus, the polypeptides can be used in a conventional immunoassay, including, without limitation, ELISA, RIA, LFA, FACS, tissue immunohistochemistry, Western blot or immunoprecipitation. The disclosed nanobodies can also be used in nanotechnology methods, such as microfluidic immunoassays, which can be used to capture PD-L1, or exosomes containing PD-L1. Suitable samples for use with a microfluidic immunoassay or other nanotechnology method, include but are not limited to, saliva, blood, and fecal samples. Microfluidic immunoassays are described in U.S. Patent Application No. 2017/0370921, 2018/0036727, 2018/0149647, 2018/0031549, 2015/0158026 and 2015/0198593; and in Lin et al., JALA June 2010, pages 254-274; Lin et al., Anal Chem 92: 9454-9458, 2020; and Herr et al., Proc Natl Acad Sci USA 104(13): 5268-5273, 2007, all of which are herein incorporated by reference).

The following examples are provided to illustrate certain particular features and/or embodiments. These examples should not be construed to limit the disclosure to the particular features or embodiments described.

### EXAMPLES

The following Examples describe a semi-synthetic shark V_{NAR} phage library based on randomization of CDR3, which was successfully used to isolate several anti-PD-L1 specific nanobodies. PD-L1-targeted nanobody-based CAR T cells were demonstrated to be effective in treating GPC3-positive solid tumors in animal models of triple-negative breast cancer and liver cancer.

### Example 1: Materials and Methods

This example describes the materials and experimental methods for the studies described in Examples 2-9.

### Cell Culture

Human breast cancer cell line MDA-MB-231, human ovarian cancer (OC) cell lines IGROV-1, OVCAR8 and NCI-ADR-RES, human pancreatic cancer (PDAC) cell lines KLM1, Panc-1, and SU8686), and lung cancer cell lines L55, EKVX, and H522 were purchased from American Type Culture Collection (ATCC). The MDA-MB-231 cell line was transduced with a lentiviral vector encoding a GFP-firefly-luciferase (GFP-Luc). The PD-L1 knockout (KO) MDA-MB-231 cell line was constructed using the clustered regularly interspaced short palindromic repeats (CRISPR)/CRISPR-associated protein 9 (CRISPR-Cas9) method. The construct was generated following the design principle as described previously (Li et al., Hepatology 2019; 70(4):1231-1245). Briefly, two single-guide RNAs (sgRNAs) targeted to the endogenous PD-L1 promotor (predicted from the EPD database) were designed and used to subclone into a LentiCRISPRv2 vector according to the manufacturer's instructions and sorted to generate single clones. Hep3B GFP-Luc was established in a previous study. MDA-MB-231 and Hep3B cells were cultured in DMEM supplemented with 10% FBS, 1% L-glutamine, and 1% penicillin-streptomycin and other aforementioned cell lines were cultured in RPMI. Cells were maintained in a humidified atmosphere containing 5% CO₂ at 37° C. PBMCs were isolated from peripheral blood of healthy donors using Ficoll (GE Healthcare) according to the manufacturer's instructions.

### Construction of a Synthetic 18AA CDR3 Nurse Shark V_{NAR} Phage Library

A new synthetic 18AA CDR3 nurse shark V_{NAR} phage library was constructed on the basis of a previous naïve shark library (Li et al., Proc Natl Acad Sci USA 2017; 114(32):E6623-E6631; English et al., Antib Ther 2020; 3(1):1-9; Feng et al., Antib Ther 2019; 2(1):1-11). For the V_{NAR} DNA cassettes, a non-canonical cysteine in CDR1 was mutated to tyrosine (C29Y) using naïve shark library V_{NAR} pComb3x plasmid as the template. A pair of randomized 18AA CDR3 were designed to amplify the CDR3 loop in the C29Y mutated template plasmid using the PCR method. Additionally, 20 μl of the linear PCR product was circularized by intra-molecular self-ligation in 1 ml of ligation buffer using T4 DNA ligase (New England Biolabs, Ipswich, MA). Finally, the ligation products were purified by removing the enzymes and transformed into 500 μl of electroporation competent TG1 cells (Lucigen, Middleton, WI) to make the library.

### Phage Panning Method

The phage panning protocol has been described previously (Feng et al., Antib Ther 2019; 2(1):1-11; Ho et al., J Biol Chem 2005; 280(1):607-617). The mouse PD-L1 protein from R&D systems was used for four rounds of panning. Briefly, Nunc 96-well Maxisorp plate (Thermo Scientific) was coated with 100 μg/ml PD-L1 in PBS overnight at 4° C. The plate was blocked with 2% bovine serum albumin in PBS for 1 hour at room temperature. Then 10¹⁰-10¹¹ cfu of pre-blocked phage supernatant in blocking buffer was added to each well for 1 hour at room temperature to allow binding. The bound phages were eluted with 100 μl pH 2.0 elution buffer at room temperature after four washes with PBS containing 0.05% Tween-20. The eluate was neutralized with 30 μl of 1 M Tris-HCl buffer (pH 8.5) and was used to infect freshly prepared E. coli TG1 cells. After four rounds of panning, single colonies were picked and identified by using phage ELISA.

### ELISA

The phage ELISA was performed as previously described (Feng et al., Antib Ther 2019; 2(1):1-11). Briefly, a Nunc 96-well Maxisorp plate was coated with 5 μg/ml antigenic proteins, including mouse PD-L1-His, mouse PD-L1-hFc, human PD-L1-His, human PD-L1-hFc, and the irrelevant antigen human IgG and PBS in 50 μl PBS per well overnight at 4° C. The plate was blocked with 2% BSA in PBS for 1 hour at room temperature. Pre-blocked phage supernatant was then added into the plate. The binding activity was determined with a horseradish peroxidase (HRP)-conjugated mouse anti-M13 antibody (GE Healthcare). To detect whether shark binder B2 is specific to human PD-L1, and not B7-H3, the antigenic proteins human PD-L1 and human B7-H3 were coated into the 96-well Maxisorp plate. PD-L1 specific binder B2 and B7H3 specific binder B3H1 were used to measure the binding affinity and specificity to PD-L1 and B7H3.

### Antibody Production and Purification

The soluble antibody protein was produced and purified as previous described (Feng et al., Antib Ther 2019; 2(1):1-11). Briefly, the coding sequences of PD-L1 specific V_{NAR} binders in the pComb3x phagemids were transformed into HB2151 E. coli cells. The formed colonies were pooled for culture in 2 L 2YT media containing 2% glucose, 100 μg/ml ampicillin at 37° C. until the OD600 reached 0.8-1. Culture media was then replaced with 2YT media containing 1 mM IPTG (Sigma), 100 μg/ml ampicillin, and shaken at 30-C overnight for soluble protein production. Bacteria pellet was spun down and lysed with polymyxin B (Sigma) for 1 h at 37° C. to release the soluble protein. The supernatant was harvested after lysis, and purified using HisTrap column (GE Healthcare) using AKTA.

### Peptide Scanning of Anti-PD-L1 V_{NAR}-Recognized Epitope

To predict the binding epitope of anti-PD-L1 nanobodies, a total of 24 peptides were designed based on the hPD-L1 ECD amino acid sequence. Each synthesized peptide is 18 amino acids in length with 9 amino acids of overlap, which allows the minimum antigenic region of hPD-L1 ECD recognized by each V_{NAR} to be narrowed down by step-by-step peptide mapping onto a 9-mer peptide epitope. The ELISA was performed as previously described. In brief, the 24 peptides were coated into individual wells of a 96-well Maxisorp plate. Five μg/ml B2-His-flag, A11-His-flag, or F5-His-flag were then added into the plate followed a horseradish peroxidase (HRP)-conjugated anti-flag antibody. The binding activity was determined by ODA450. Experiments were performed in triplicate and repeated three times with similar results.

### Shark PD-L1-Target V_{NAR}-Based CAR T Lentiviral Construction and Generation

Shark PD-L1-target V_{NAR}-based CAR T lentiviral vector was constructed following the design principle of the CAR construct published previously (Li et al., Gastroenterology 2020; 158(8):2250-2265), replacing the hYP7 scFv with PD-L1-target V_{NAR} fragment (B2 and A11) as the PD-L1 recognition molecule. The constructs have lentiviral expressing vector pWPT (Addgene #12255) as the backbone, and were engineered with expression cassettes encoding CD8α hinge and transmembrane regions, 4-1BB and CD3ζ signaling domains, the self-cleaving T2A ribosomal skipping sequence, and a truncated human epidermal growth factor receptor (hEGFRt). The CAR expressing lentivirus were produced as described previously (Li et al., Gastroenterology 2020; 158(8):2250-2265).

### CAR T Cell Production

Active T cells from healthy donor PBMCs were obtained upon stimulation with anti-CD3/anti-CD28 antibody-coated beads (Invitrogen) at a bead to cell ratio of 2:1 for 24 hours in complete RPMI media with IL-2. Active T cells were transduced with PD-L1-target V_{NAR} CAR lentivirus. CAR T cells were expanded for 7 days and subsequently used for in vitro and in vivo assays.

### Flow Cytometry

Surface PD-L1 expression was detected by anti-PD-L1 monoclonal antibody (Biolegend) and goat-anti-human IgG-phycoerythrin (PE) (Jackson ImmunoResearch). The transduction efficiency of PD-L1-targeted CAR T cells was detected by surface hEGFRt expressing using flow cytometry. The cells were incubated with anti-EGFR human monoclonal antibody cetuximab (Erbitux) and goat-anti-human IgG-PE (Jackson ImmunoResearch) to measure the efficiency of binding on transduced CAR T cells.

### In Vitro Cytolysis of CAR T Cells and Activation Assays

Cytotoxicity of CAR T cells was determined by a luciferase-based assay. In brief, the luciferase-expressing MDA-MB-231 and Hep3B tumor cells were used to establish a cytolytic assay. The cytolysis of PD-L1-target CAR T cells was detected by co-culture with MDA-MB-231 GFP-Luc and Hep3B GFP-Luc at various E:T ratios for 24 hours or 96 hours followed by measurement of the luciferase activity using the luciferase assay system (Promega) on Victor (PerkinElmer). The supernatants were collected from each co-culture and used for TNF-α, IL-2, and IFN-γ detection using ELISA Kit (BD biosciences). In the killing blocking assay of CAR T cells, varying concentration of soluble B2 nanobody was added into the setup cytotoxicity system of tumor cells by incubation for 24 hours and 48 hours.

### Western Blot

Cells were lysed with ice-cold lysis buffer (Cell Signaling Technology), and total protein was isolated by centrifugation at 10,000 g for 10 minutes at 4° C. Protein concentration was measured using a Bicinchoninic acid assay (Pierce) in accordance with the manufacturer's specifications. Twenty μg of each cell lysate were loaded onto a 4-20% SDS-PAGE gel for electrophoresis. Both anti-PDL1 antibody and the anti-GAPDH antibody were obtained from Cell Signaling Technology.

### Animal Studies

Five-week-old female NOD/SCID/IL-2Rgc^{null} (NSG) mice (NCI Frederick) were housed and treated under a protocol approved by the Institutional Animal Care and Use Committee at the NIH. For the orthotopic MDA-MB-231 model, mice were inoculated with 3 million MDA-MB-231-GFP-Luc cells suspended in the mixture of PBS:Matrigel (BD Biosciences) at 1:1 in the inguinal mammary fat pad. Mice with established tumors were randomly allocated into two groups and intravenously (i.v.) infused once with 5 million CAR (B2) T cells or relevant control CAR (CD19) T cells. The peritoneal Hep3B xenograft tumor model was established as previously described (Li et al., Gastroenterology 2020; 158(8):2250-2265). In short, 3 million Hep3B-GFP-Luc cells suspended into PBS were intraperitoneally (i.p.) injected into mice. After 12 days post tumor inoculation, 10 successfully implanted mice were randomly allocated into two groups followed by i.p. infusion once of 5 million CAR (B2) T cells or CAR (CD19) T cells. Tumors were measured by total bioluminescent intensity using a Xenogen IVIS Lumina (PerkinElmer) weekly and tumor size of orthotopic MDA-MB-231 was calculated using the formula ½ (length×width²) for digital caliper measurements. Spleens of sacrificed mice were dissociated using a Miltenyi Biotec tumor dissociation kit and the obtained immune cells were cultured in vitro. The isolated T cells were then stained for CAR expression using flow cytometry and ex vivo killing was detected.

### Example 2: Construction of a Semi-Synthetic Nurse Shark V_{NAR} Library

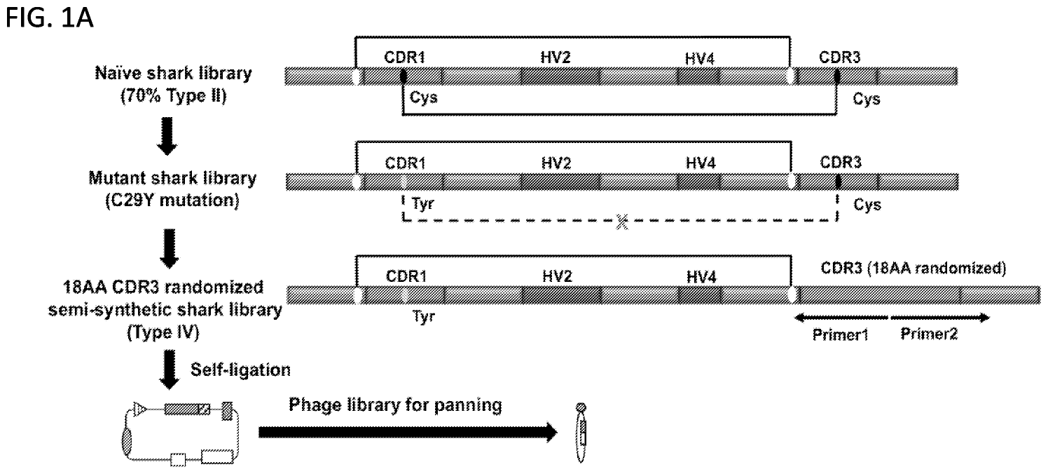

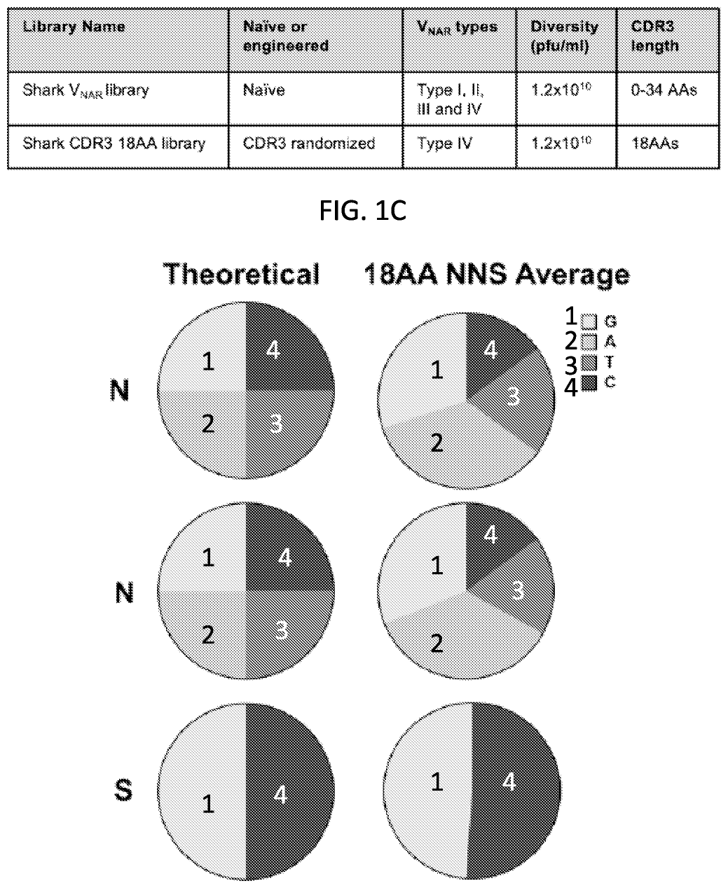

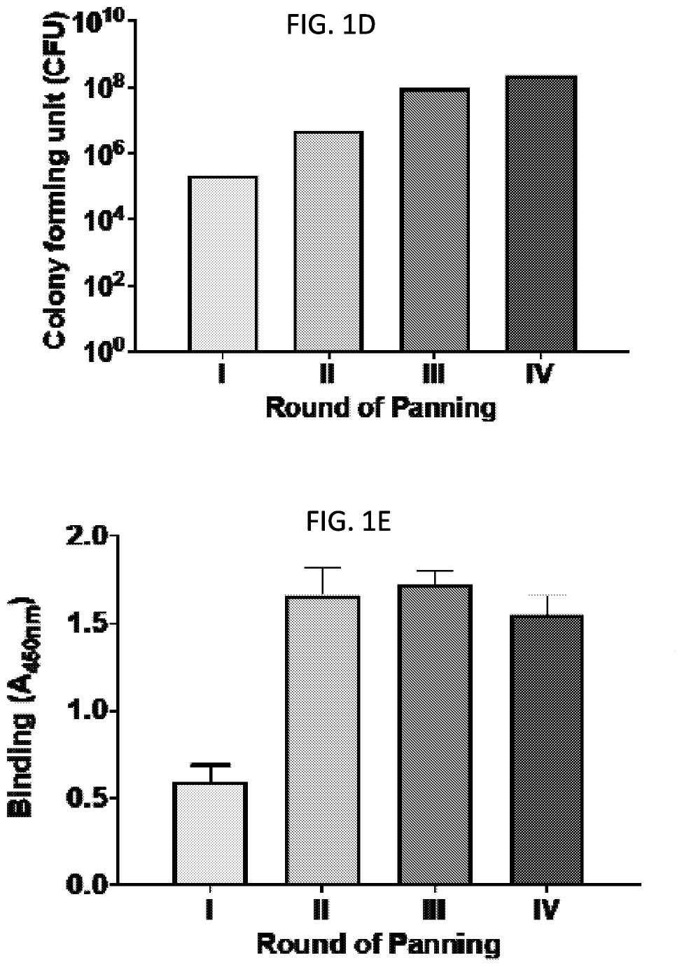

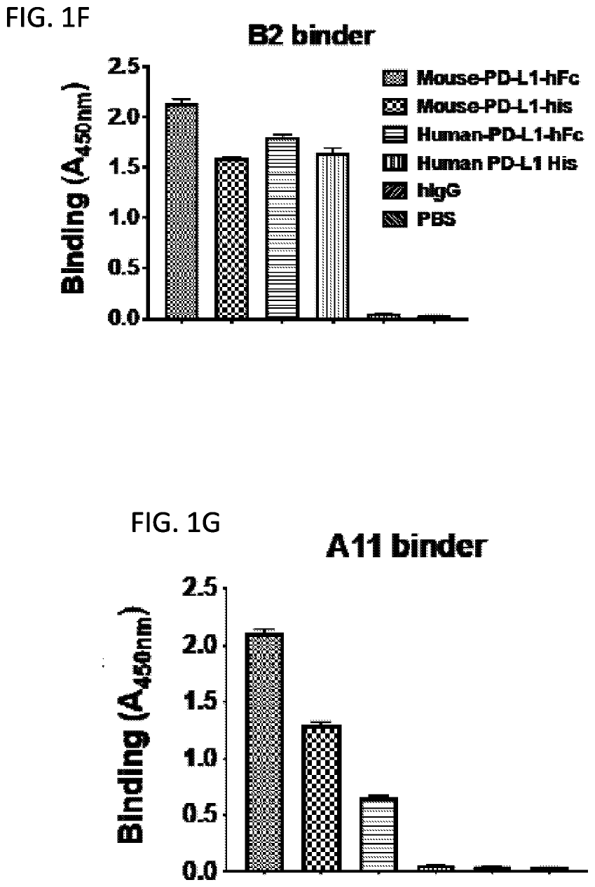

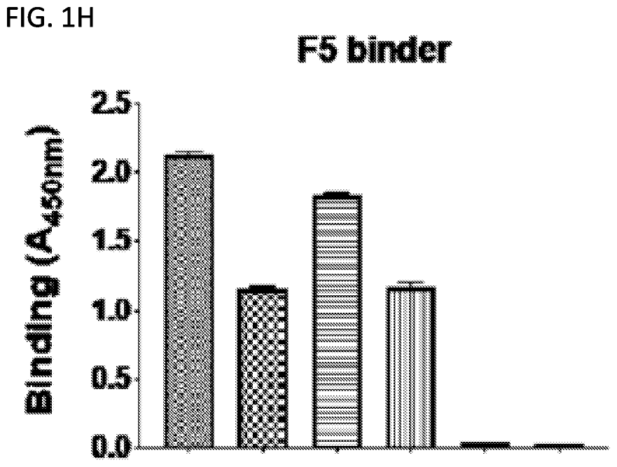

A naïve nurse shark V_{NAR} library was previously constructed from 6 naïve adult nurse sharks (Ginglymostoma cirratum) with a size of 1.2×10 pfu/ml (Li et al., Proc Natl Acad Sci USA 2017; 114(32):E6623-E6631; English et al., Antib Ther 2020; 3(1):1-9; Feng et al., Antib Ther 2019; 2(1):1-11). In the present study, to improve the abundance and utility of the shark V_{NAR} library, a semi-synthetic randomized CDR3 18AA shark V_{NAR} library (referred to ‘18AA CDR3 shark library’) was generated. As illustrated in FIG. 1A, 70% of V_{NAR}, in the naïve shark library are Type II containing two canonical cysteines located at amino acid 21 and 82 to form a disulfide bond and at least one extra cysteine in CDR1 and CDR3 to from an interloop disulfide bond. These atypical disulfide bonds are believed to be essential for stabilization of IgNAR proteins and can affect the structure of the antigen-binding surface. The C29Y mutation and randomized CDR3 loop region changed all V_{NAR}, to type IV instead of four classical types (Type I, II, III, and IV) and maintained their diversity at 1.2×10¹⁰ pfu/ml compared with the naïve shark V_{NAR} library (FIGS. 1A and 1B). To assess the randomness of sequences modification, the average nucleotide ratio at each randomization NNS was estimated based on sequencing analysis, and it was found that the CDR3 nucleotides were completely randomized with desired ATGC bases ratios (FIG. 1C).

### Example 3: Isolation of V_{NAR} Binders Specific to Mouse PD-L1

To identify the anti-PD-L1 shark V_{NAR} binders that could function in the murine tumor environment, mouse PD-L1 protein was used as an antigen to screen the new semi-synthetic shark library. After four rounds of panning, approximately 1,000-fold enrichment of eluted phage colonies was obtained (FIG. 1D). An enhanced binding to PD-L1 was also observed after two rounds of phage panning (FIG. 1E). At the end of the fourth round of panning, 46 individual clones were identified to bind mouse PD-L1 (mPD-L1) protein by monoclonal phage ELISA, and 12 unique binders (B2, F5, A11, A3, A9, A2, A10, A7, A6, C4, A1 and D12, respectively set forth herein as SEQ ID NOs: 1-12) were confirmed by subsequent sequencing. Three PD-L1-specific binders, B2, A11, and F5, showed cross-activity to both mouse and human PD-L1 (hPD-L1) protein with the His-tag or the hFc-tag formats, as shown by monoclonal phage ELISA (FIG. 1F-H).

### Example 4: Activity of PD-L1-Specific Single Domain V_{NARs}

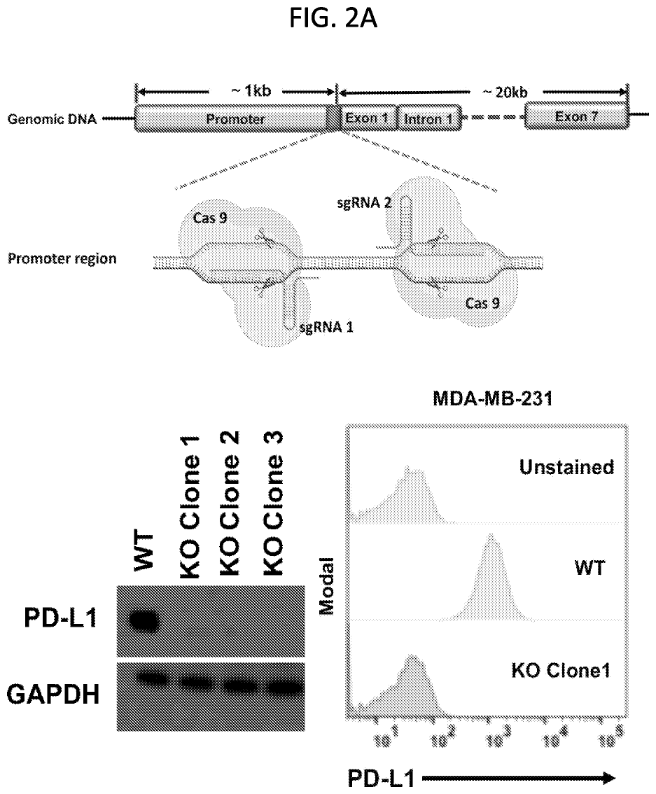

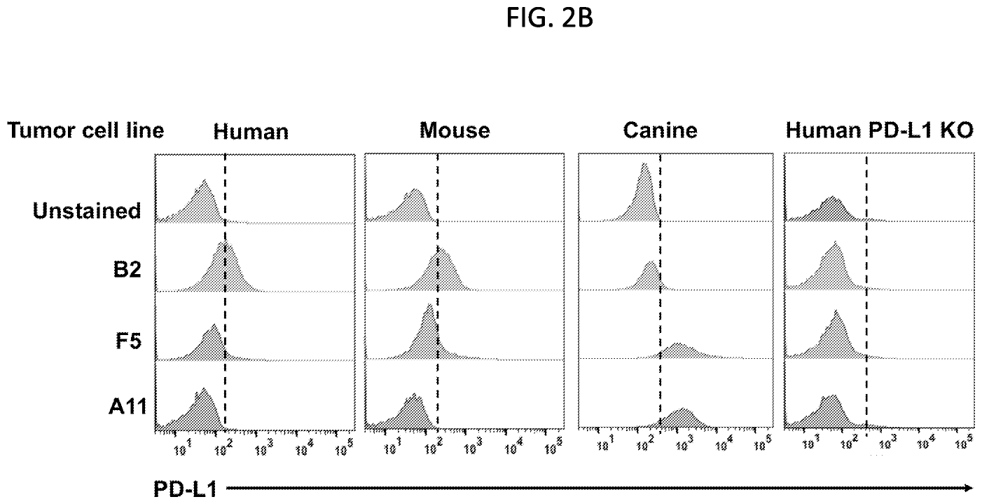

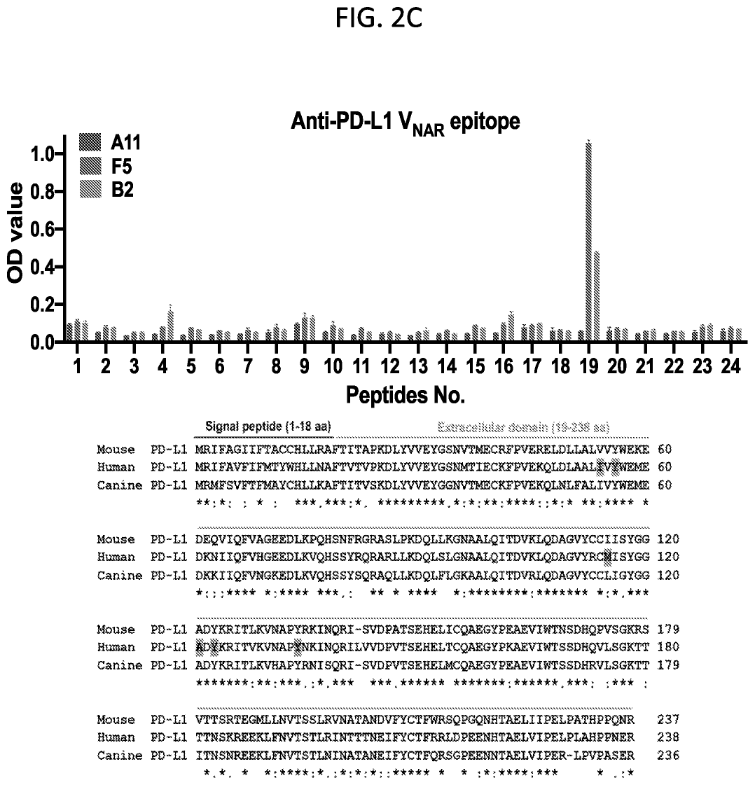

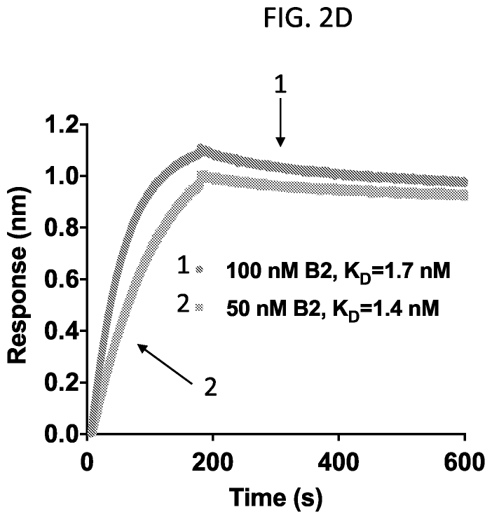

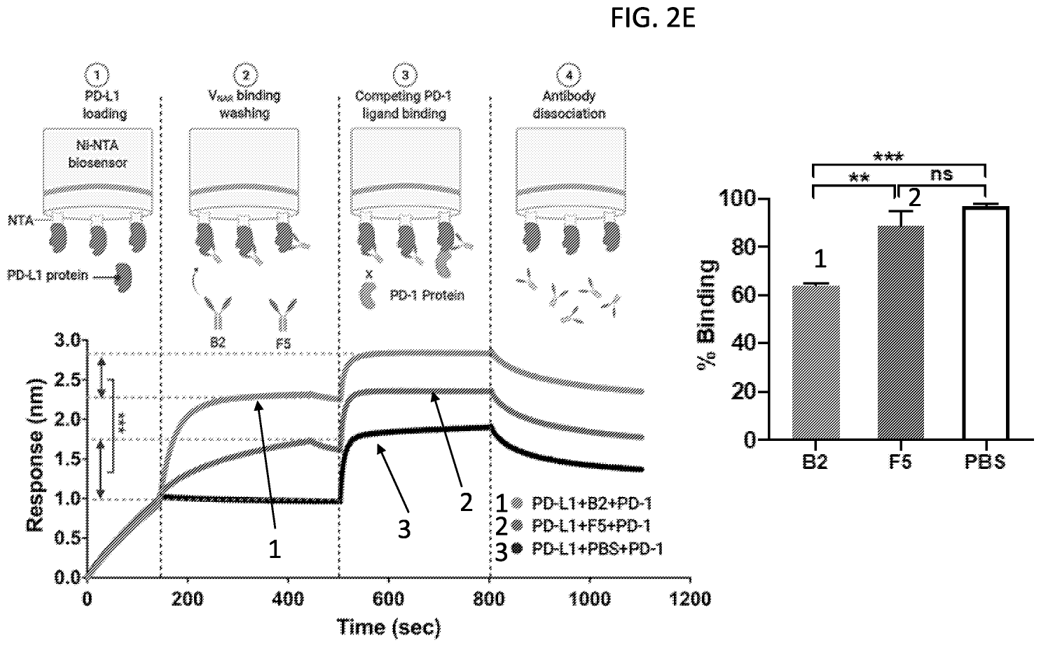

To determine the antigen specificity of shark V_{NARs}, a PD-L1 knockout (KO) single clones were established using CRISPR-Cas9 technology in a human TNBC cell line, MDA-MB-231. To enhance the PD-L1 KO efficiency, two single guide RNAs (sgRNAs) were designed to target the promoter of the endogenous PD-L1 gene (FIG. 2A). All three individual cell clones were confirmed by loss of PD-L1 expression (FIG. 2A), and clone 1 was used further in the present study. To determine the cross-species reactivity of anti-PD-L1 shark V_{NAR}s against native PD-L1, three PD-L1-positive tumor cell lines, including a human breast cancer cell line, a mouse melanoma cell line, and a canine melanoma cell line, were used to evaluate the binding ability of B2, A11, and F5. As shown in FIG. 2B, B2 and F5 bind human antigens and cross-react with mouse and canine antigens. B2 showed a higher binding ability to human and mouse antigens than F5. A11 binds canine antigen but not human or mouse antigen. In contrast, no binding was shown on PD-L1 KO cells, indicating that the binding ability of shark V_{NAR}s is antigen specific.

V_{NAR}-hFc fusion proteins were also produced and incubated with hPD-L1-His protein on the biolayer interferometry (BLI) Octet platform to determine the binding kinetics. The K_{D} value of B2 was 1.7 nM and 1.4 nM at a concentration of 100 nm and 50 nM, respectively (FIG. 2C), whereas F5 failed to yield an accurate K_{D} value because it showed slight non-specific binding to the nickel-charged tris-nitrilotriacetic acid (Ni-NTA) sensor on Octet. To examine whether B2 could functionally block the interaction between human PD-1 (hPD-1) and hPD-L1, a blocking assay was developed based on BLI technology. As shown in FIG. 2D, B2 partially blocked the interaction of hPD-1 with hPD-L1 compared with the PBS control. In contrast, F5 showed positive binding to hPD-L1 but could not block the hPD-1/hPD-L1 interaction. A sandwich ELISA was conducted to validate the functional blocking capacity of B2. It showed that the V_{NAR} did partially block the interaction of hPD-1 with hPD-L1 (FIG. 2E). In addition, V_{NAR} B2 specifically binds to hPD-L1 but not human B7-H3, another B7-CD28 family member (FIG. 2F).

To further explore the binding epitope of anti-PD-L1 nanobodies, a peptide array was synthesized based on the sequence of the hPD-L1 extracellular domain (ECD) that consisted of a total of 24 peptides. As shown in FIG. 2G, nanobodies F5 and B2 strongly bound to peptide #19 (TTNSKREEKLFNVTSTLR; SEQ ID NO: 13), whereas A11 didn't bind to any peptides.

These results demonstrate the identification of functionally cross-species anti-PD-L1 shark V_{NAR} with high affinity.

### Example 5: In Vitro Activity of Anti-PD-L1 Nanobody-Based CAR T Cells on MDA-MB-231 Tumor Cells

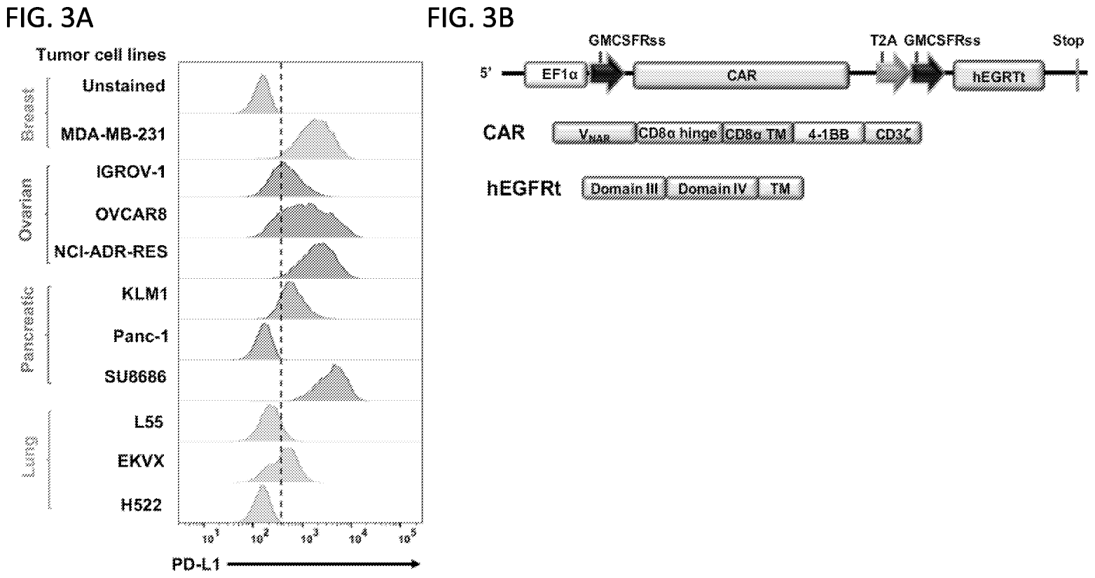

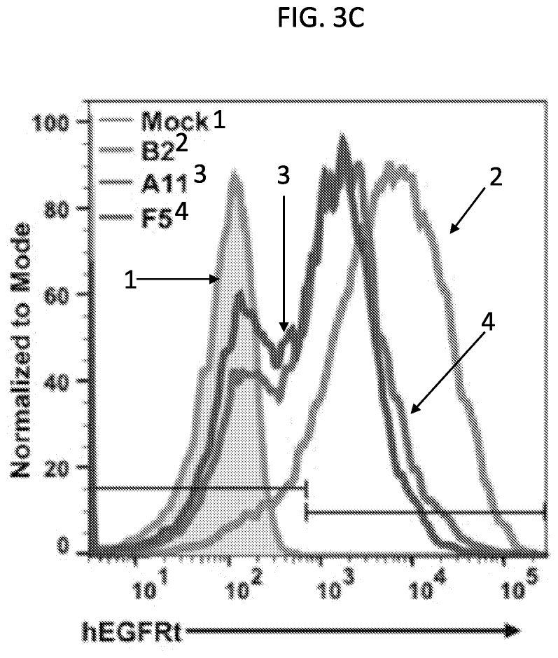

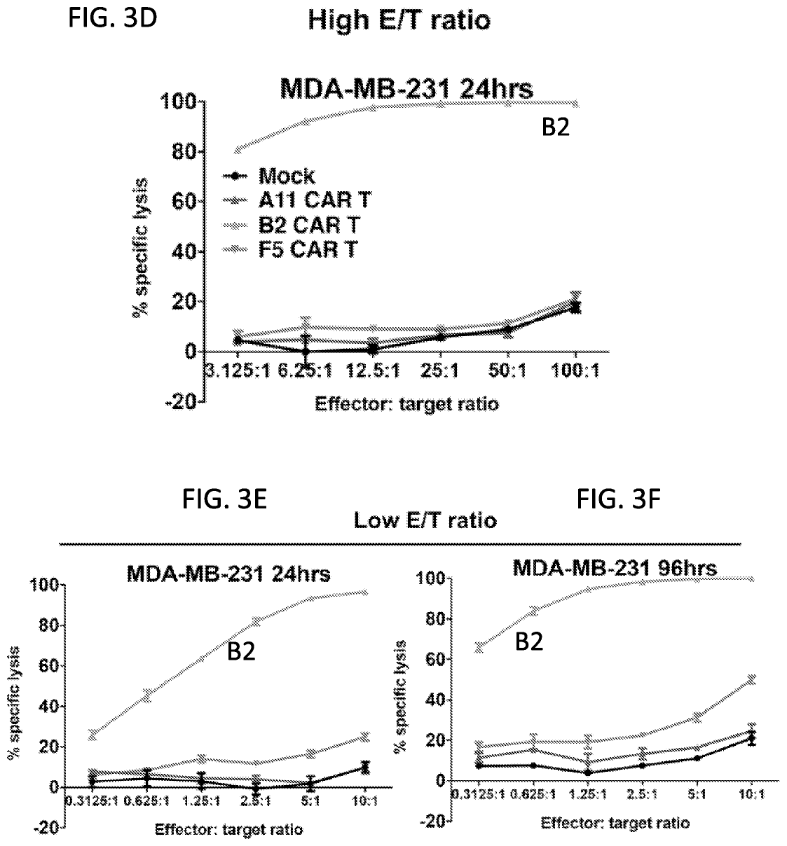

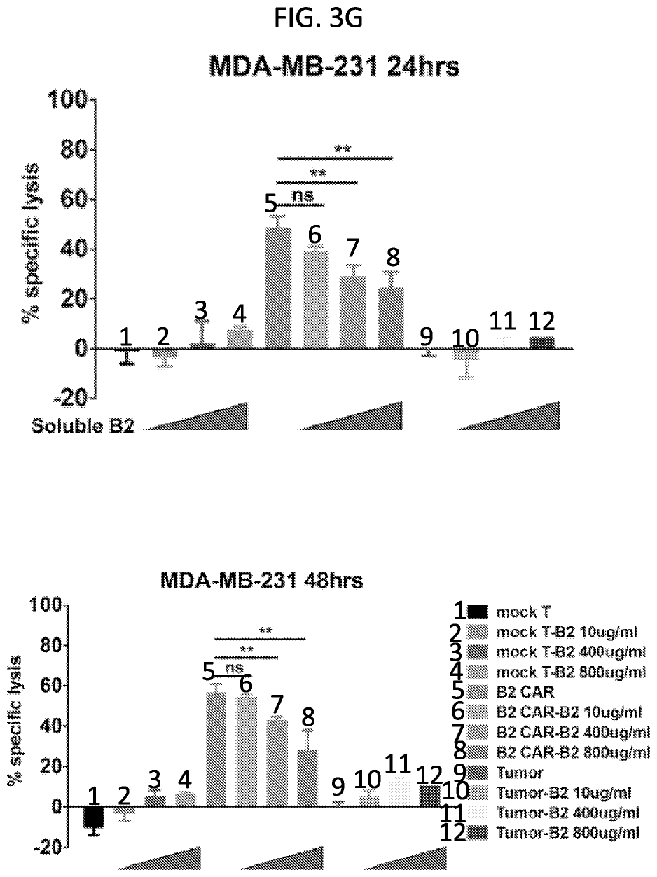

Using flow cytometry, it was determined that PD-L1 was overexpressed in all four tested human tumor types, including breast cancer cell line MDA-MB-231, three ovarian cancer cell lines (IGROV-1, OVCAR8, and NCI-ADR-RES), two out of three pancreatic cancer cell lines (KLM1 and SU8686), and the lung cancer cell line EKVX, indicating that PD-L1 could serve as a pan-cancer target (FIG. 3A). MDA-MB-231 is a highly aggressive, invasive, and poorly differentiated TNBC cell line with limited treatment options. The anti-PD-L1 nanobody-based CAR T cells were generated according to the design of the previously described scFv-based GPC3-targeted CAR T cells in liver cancer (Li et al., Gastroenterology 2020; 158(8):2250-2265). Briefly, the GPC3-specific scFv was replaced with one of three V_{NAR} fragments (anti-PD-L1 binders B2, A11 or F5) as the recognition region, along with 4-1BB, and CD3ζ signaling domains and a truncated human EGFR cassette to gauge transduction efficiency and to switch CAR off (FIG. 3B). T cells obtained from healthy donor PBMCs were activated with anti-CD3/CD28 beads followed by CAR lentivirus transduction and incubated with IL-2. The transduction efficiency of CAR (B2) T cells and CAR (F5) T cells is about 90%, while CAR (A11) T cells had a transduction efficiency of 64% (FIG. 3C). To examine whether anti-PD-L1 nanobody-based CAR T cells could recognize and lyse the tumor cells, a luciferase-based cytolytic assay was established. Three individual nanobody-based CAR T cells were incubated with MDA-MB-231 cells at a high Effector:Target (E:T) ratio (maximum 100:1) for 24 hours, or at a low E:T ratio (minimum 0.3125:1) for 24 hours or 96 hours in an E:T ratio-dependent manner. As shown in FIG. 3D, MDA-MB-231 cells were effectively lysed by CAR (B2) T cells in a 2-fold dose-dependent manner at both high and low E:T ratios. Moreover, the long incubation time of 96 hours could efficiently increase the cytotoxicity of CAR (B2) T cells compared with 24-hour incubation at a low E:T ratio. In contrast, minimal cell lysis was found in MDA-MB-231 cells when incubated with CAR (A11) T, CAR (F5) T, and mock T cells. A significantly higher level of TNF-α, IL-2, and IFN-γ was released from CAR (B2) T cells when co-cultured with MDA-MB-231 at different E:T ratios, while minimum cytokine production was observed from CAR (A11) T and CAR (F5) T (FIG. 3E). These results suggested that PD-L1-targeted CAR (B2) T cells were able to efficiently lyse MDA-MB-231 tumor cells in vitro.

### Example 6: Killing of PD-L1-Specific Shark Nanobody-Based CAR T Cells is Antigen Specific

To investigate whether the cytolytic activity of CAR (B2) T cells is antigen dependent, CAR (B2) cells were incubated with either the MDA-MB-231 WT or MDA-MB-231 PD-L1 KO cell line (KO clone 1) at various E:T ratios for 24 hours. As shown in FIG. 3F, CAR (B2) T cells were not capable of killing PD-L1 KO cells. Furthermore, a corresponding soluble B2 V_{NAR} nanobody was included in the MDA-MB-231 co-culture setup to detect whether it could affect the cytotoxicity of CAR(B2) T cells via blocking the recognition site on tumor cells competitively. As shown in FIG. 3G, inclusion of a B2 nanobody significantly inhibited the cytolytic activity of CAR (B2) T cells after 24 or 48 hours of incubation with tumor cells. In contrast, no specific lysis of tumor cells was found with either incubation with mock T cells or tumor cells alone in the presence of B2 nanobody. Taken together, the cytotoxicity of CAR (B2) T cells is PD-L1 specific.

### Example 7: CAR (B2) T Cells Kill Inducible PD-L1+ Hepatocellular Carcinoma (HCC) Cells In Vitro and In Vivo

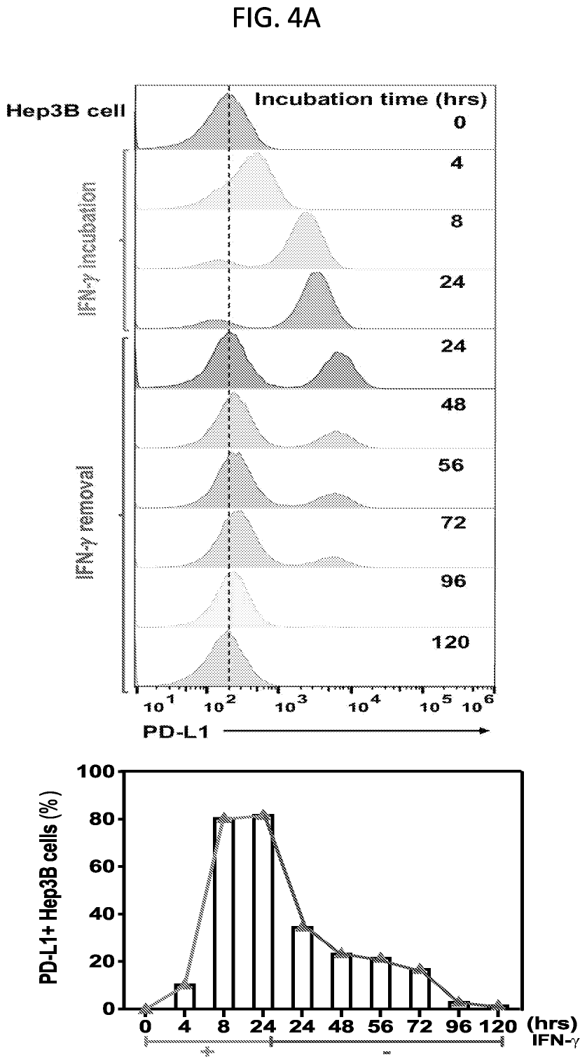

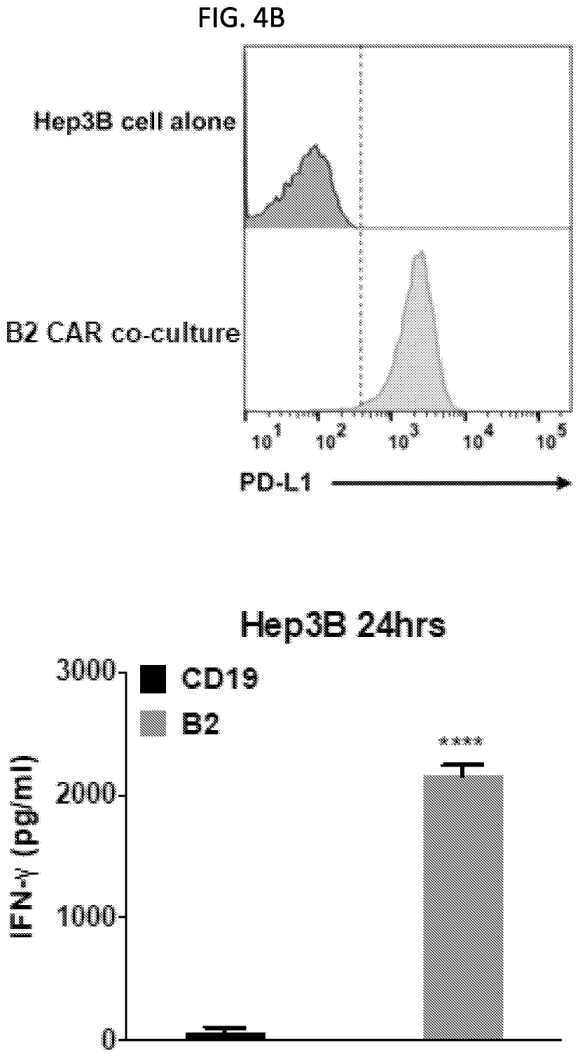

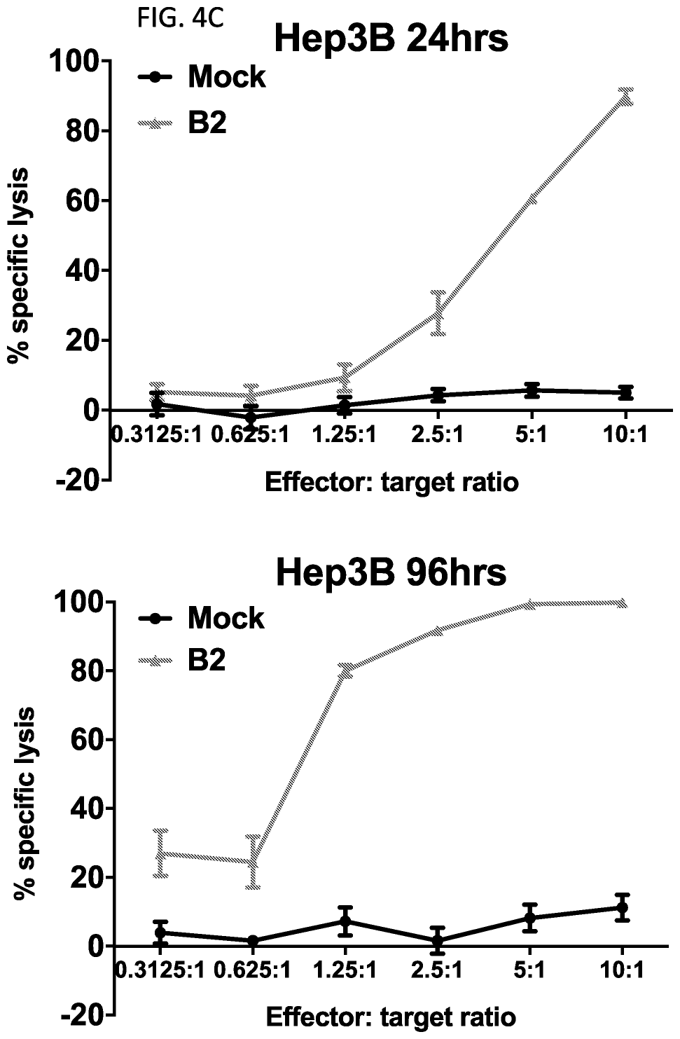

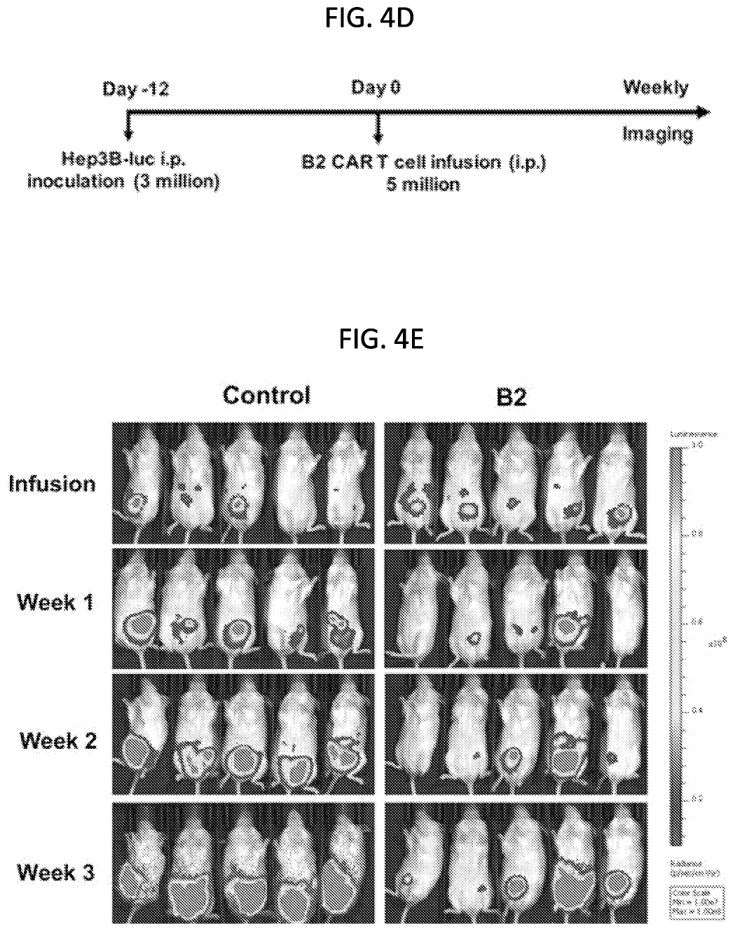

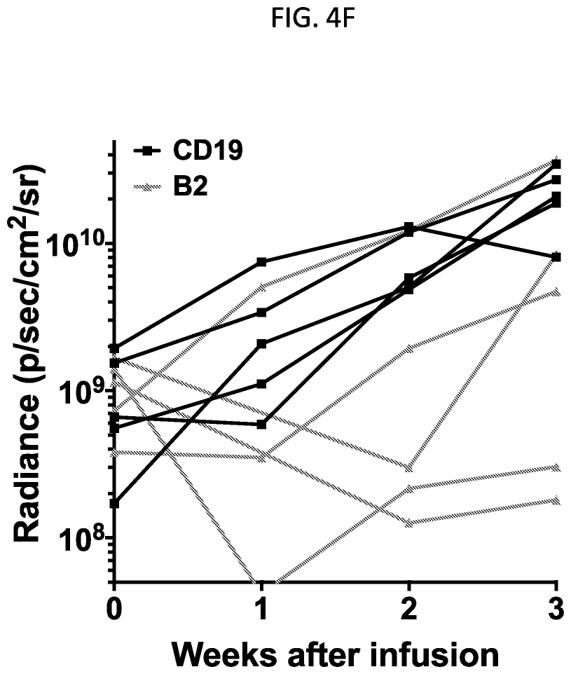

Hep3B, a hepatocellular carcinoma (HCC) cell line, does not have constitutive PD-L1 expression. However, significantly increased expression of PD-L1 was observed on Hep3B cells upon IFN-γ incubation (4 hours), which reached a peak at 8 hours and gradually decreased over time after IFN-γ removal but remained for up to 96 hours (FIG. 4A). Following 24 hours of incubation of Hep3B with CAR (B2) T cells at an E:T ratio of 0.5:1, inducible PD-L1 expression was found in Hep3B tumor cells due to the release of massive IFN-γ from CAR-T cells (FIG. 4B). To test whether anti-PD-L1 CAR (B2) T cells could kill inducible-PD-L1 tumor cells, CAR (B2) T cells were incubated with Hep3B GFP-Luc tumor cells at various E:T ratios for 24 hours and 96 hours. As shown in FIG. 4C, Hep3B GFP-Luc cells were effectively lysed by CAR (B2) T cells in a 2-fold dose-dependent manner after 24 hours and 96 hours of incubation. To further evaluate the antitumor effects of CAR (B2) T cells in HCC in vivo, the Hep3B xenograft model was used with intraperitoneal (i.p.) injection of Hep3B GFP-Luc tumor cells into the mouse as previously described (Li et al., Gastroenterology 2020; 158(8):2250-2265). After 12 days of tumor inoculation, mice were i.p. infused with 5 million CAR (CD19) T cells or CAR (B2) T cells (FIG. 4D). Four out of 5 mice treated with CAR (B2) T cells showed a statistically significant decrease in tumor growth compared with the CAR (CD19) T group (FIGS. 4E and 4F). Thus, these in vitro and in vivo results demonstrated that PD-L1-targeted V_{NAR}-based CAR (B2) T cells provide a significant benefit in HCC therapy with a moderate antitumor activity.

### Example 8: Bispecific CAR (hYP7−B2) T Cells Improve Cytotoxicity In Vitro

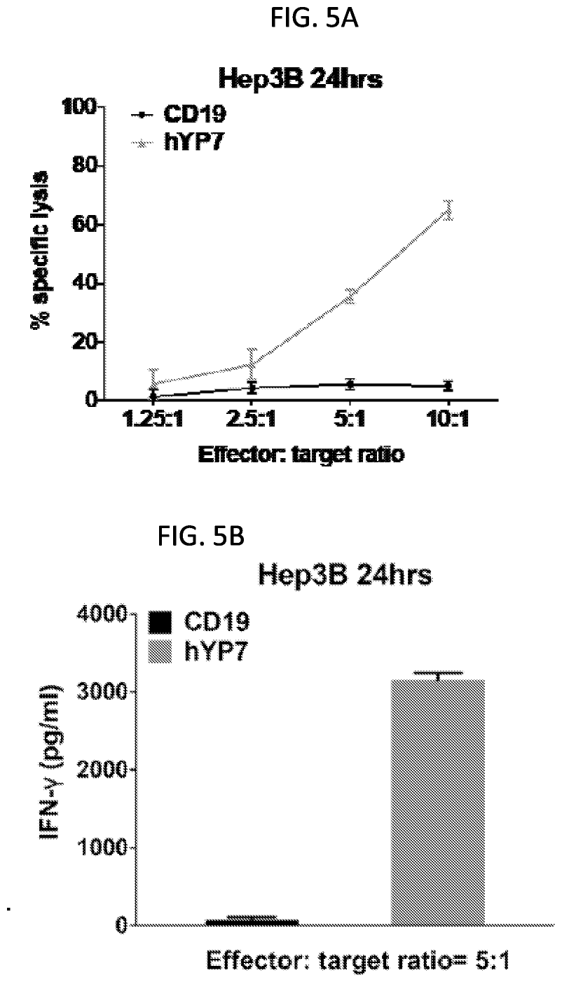

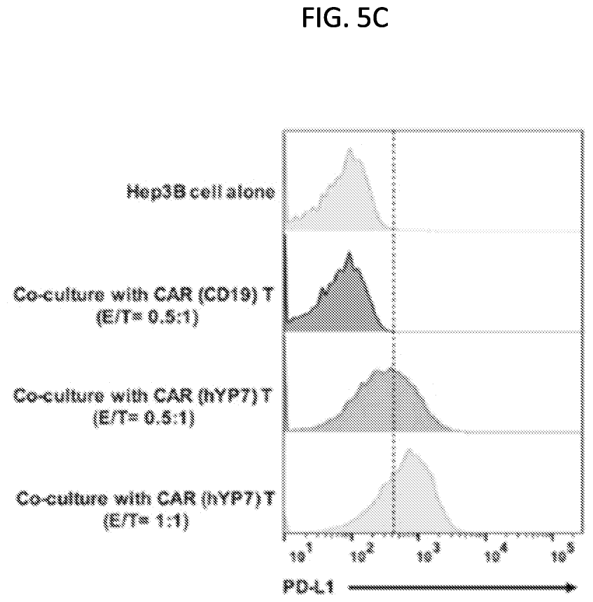

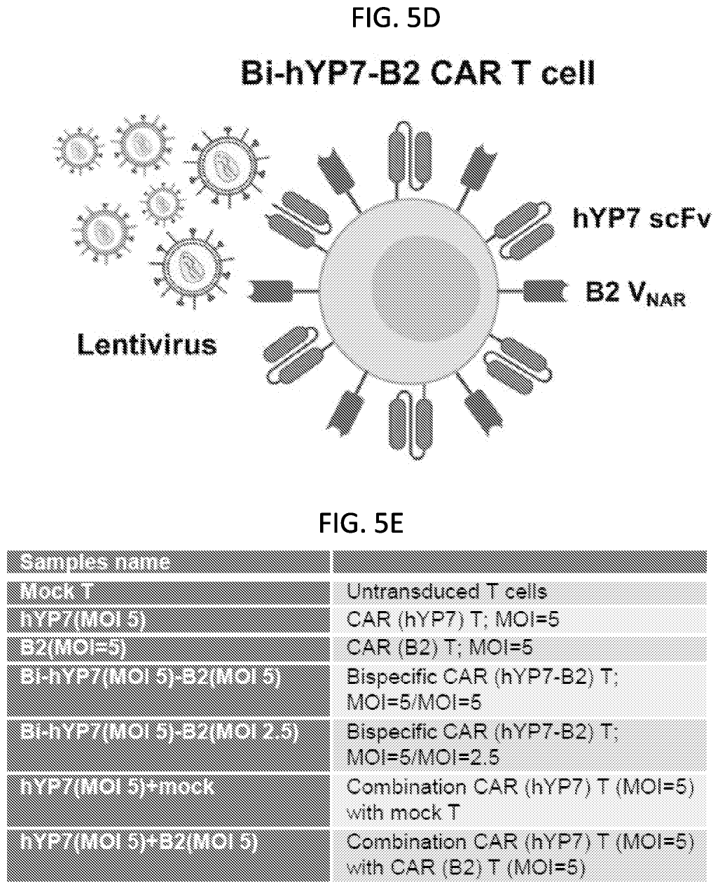

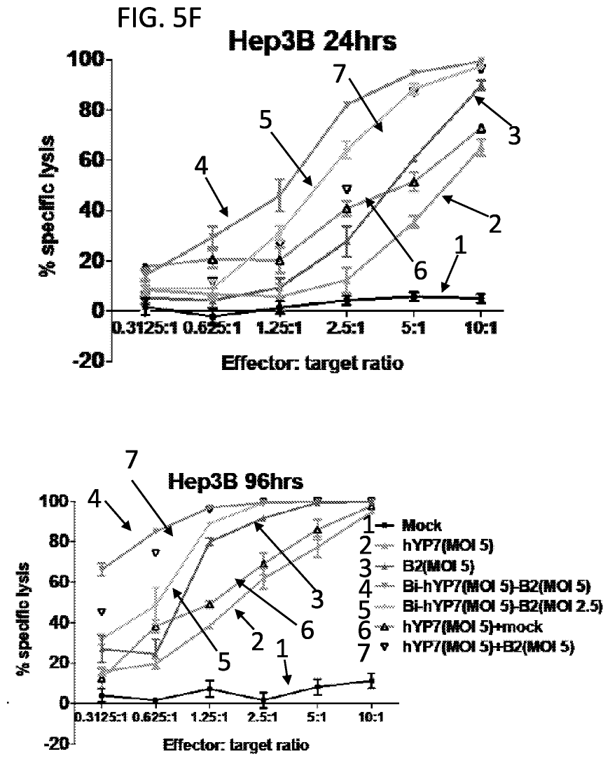

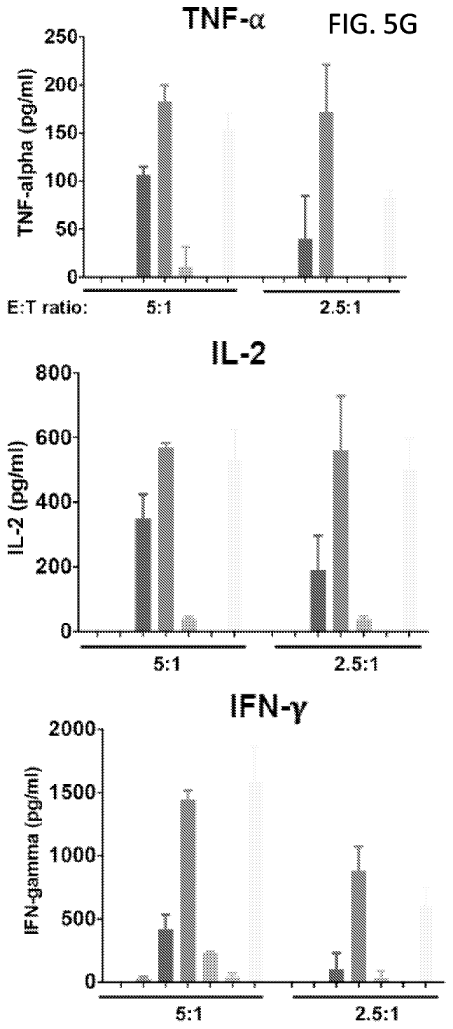

GPC3 has been suggested as an emerging tumor antigen in HCC. As shown in FIG. 5A, GPC3-targeted CAR (hYP7) T cells specifically lysed Hep3B tumor cells, which is consistent with the previous findings (Li et al., Gastroenterology 2020; 158(8):2250-2265). A significantly higher level of IFN-γ was released from CAR (hYP7) T cells compared with CAR (CD19) T cells at E:T ratio of 5:1 post 24 hours incubation (FIG. 5B). It was observed that PD-L1 expression was induced in Hep3B cells upon interaction with CAR (hYP7) T cells (FIG. 5C). In contrast, no PD-L1 expression was detectable on Hep3B cells alone or after incubation with antigen-mismatched CAR (CD19) T cells. It appears that IFN-γ produced by tumor infiltrating lymphocytes (TILs) strongly induced upregulation of PD-L1 which may allow cancers to evade the host immune system (Sharpe and Pauken, Nat Rev Immunol 2018; 18(3):153-167). Therefore, it was hypothesized that a bispecific anti-GPC3 and anti-PD-L1 CAR molecule would improve the antitumor response of T cells. Bispecific CAR (hYP7−B2) T cells that co-expressed both GPC3-targeted hYP7 scFv and PD-L1-targeted B2 V_{NAR} molecules by co-transcription of CAR (hYP7) lentivirus and CAR (B2) lentivirus (FIG. 5D) were generated. In this study, different strategies were used to compare their activity in vitro, including bispecific CAR T cells (Bi-hYP7−B2), and a combination method of CAR (hYP7) T and CAR (B2) T cells (hYP7+B2) (FIG. 5E). Moreover, CAR (B2) lentivirus was used at an MOI equal to 5 or 2.5 to produce different B2 intensities of bispecific CAR (hYP7−B2) T cells and detected their activity after 24 hour and 96 hour incubations with Hep3B cells. As shown in FIG. 5F, the cytotoxicity of both Bi-hYP7(MOI 5)−B2(MOI 5) CAR T and Bi-hYP7(MOI 5)−B2(MOI 2.5) CAR T was significantly higher than hYP7(MOI 5) CAR T and B2(MOI 5) CAR T cells, especially at the lowest E:T ratio (0.3125:1) after 96 hours incubation, suggesting that the CAR construct engineered with anti-PD-L1 specific B2 was able to improve the efficiency of CAR T cells. Bi-hYP7(MOI 5)−B2(MOI 5) CAR T showed higher cytolytic activity on Hep3B cells than 24 hour and 96 hour incubation with a combination of hYP7(MOI 5) CAR T and B2(MOI 5) CAR T cells (FIG. 5F), indicating that a produced bispecific CAR T construction has more potency than just CAR T cells combination. Furthermore, higher levels of TNF-α, IL-2, and IFN-γ production were observed after 24 hours of incubation with Bi-hYP7(MOI 5)−B2(MOI 5) CAR T than with hYP7(MOI 5) CAR T and B2(MOI 5) CAR T cells at two E:T ratios of 5:1 and 2.5:1 (FIG. 5G). Additionally, cytokine production increased at higher B2 intensity in the bispecific CAR (hYP7−B2) T cells. Therefore, it was concluded that the bispecific CAR (hYP7−B2) T cells demonstrated an enhanced antitumor activity in HCC cells in vitro.

### Example 9: Combined CAR (B2) with CAR (hYP7) T Cells Exhibit a Synergistic Anti-Tumor Effect in Vivo

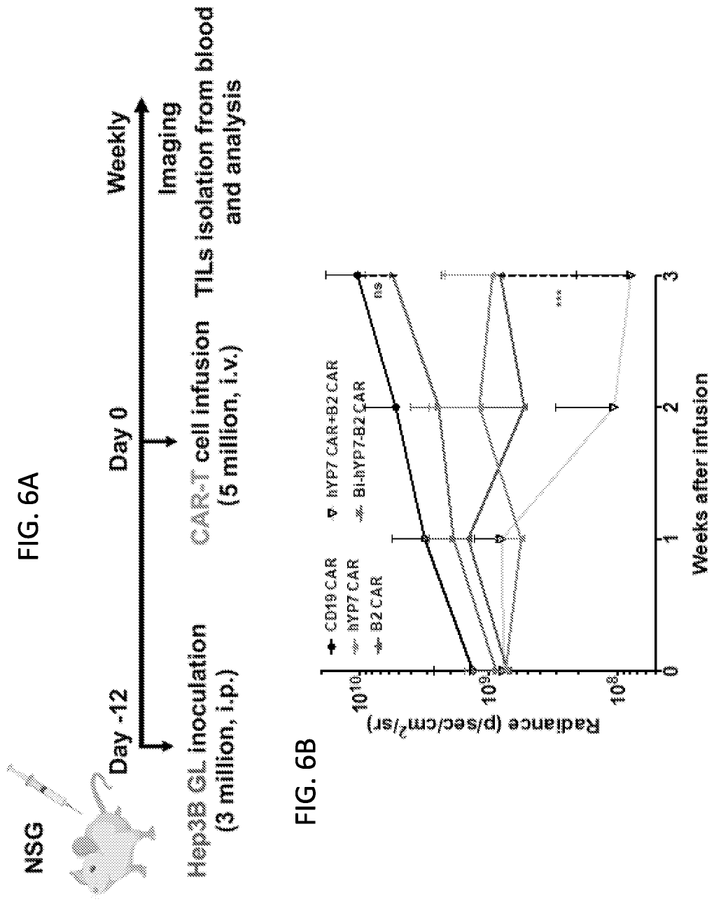

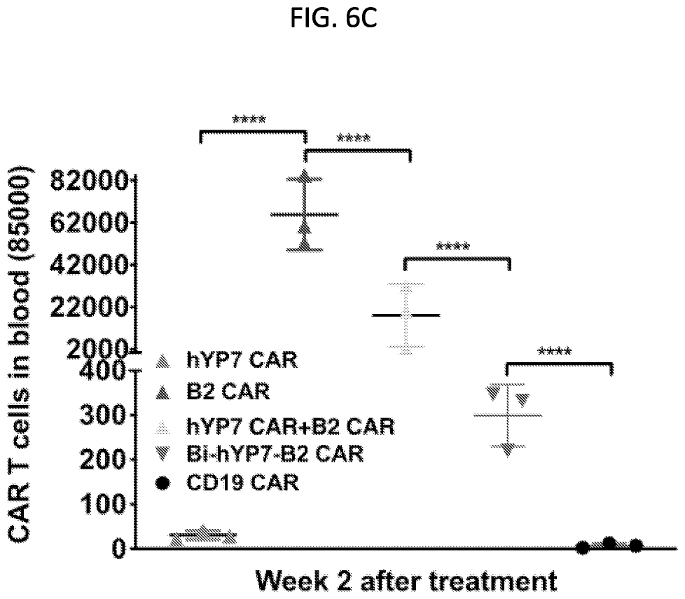

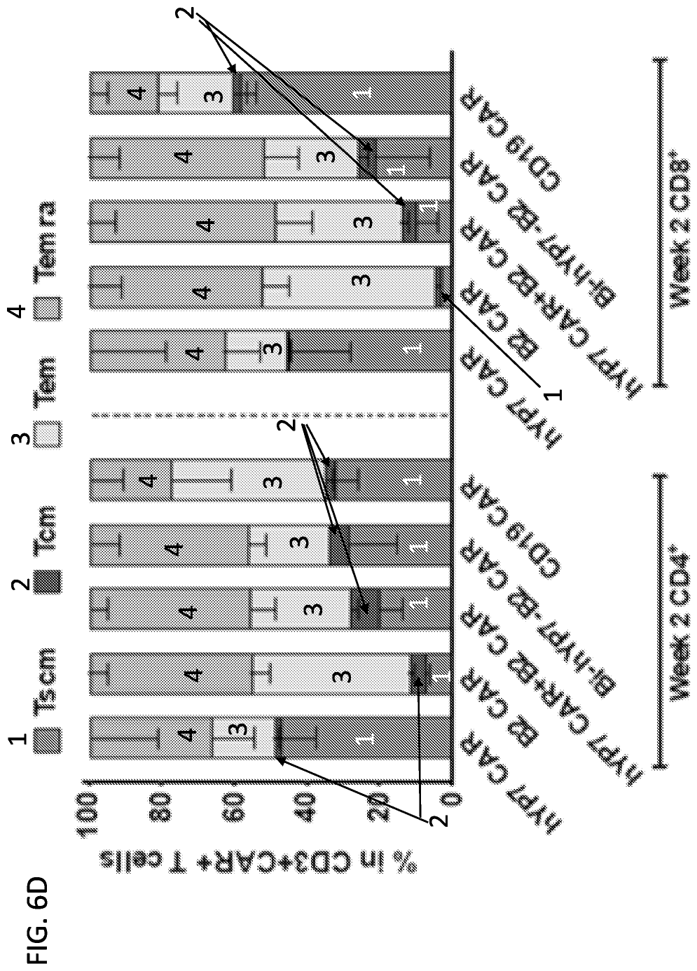

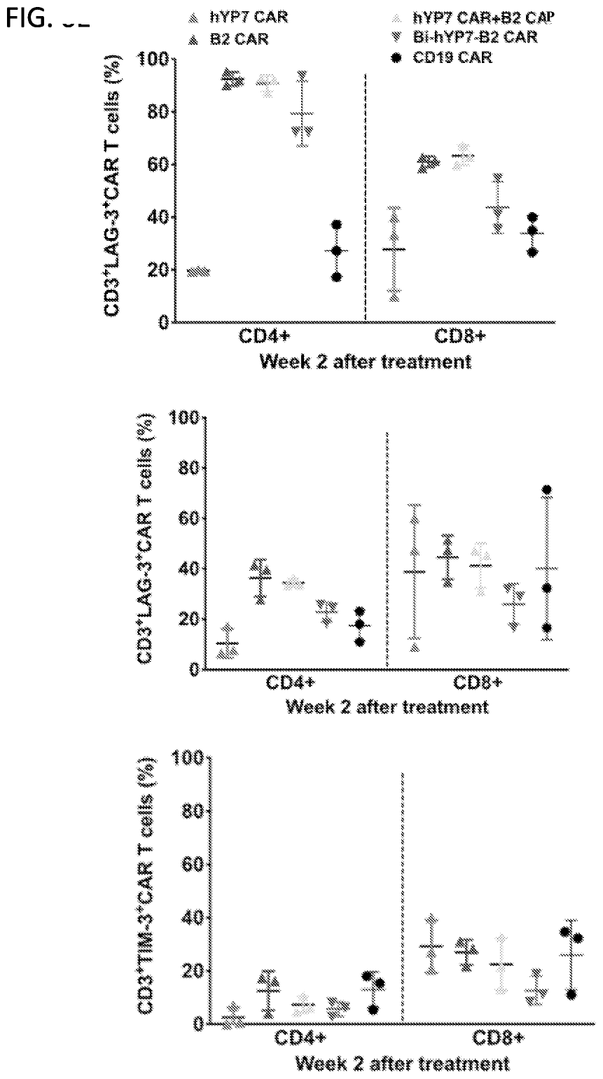

Bi-hYP7−B2 CAR T cells and the combination of hYP7 CAR T+B2 CAR T cells were tested for their ability to reduce tumor size in a liver tumor model. A schematic of the in vivo study is shown in FIG. 6A. A peritoneal Hep3B mouse model was established via i.p. injection of Hep3B GL on Day −12 followed by i.v. infusion of 5 million CAR (hYP7) T cells, CAR (CD19) T cells, CAR (B2) T cells, Bi-hYP7−B2 CAR T cells, or a combination of 2.5 million CAR (hYP7) T cells and 2.5 million CAR (B2) T cells (referred to as “hYP7 CAR+B2 CAR”) at Day 0. In comparison with CAR (CD19) T cells, both CAR (hYP7) T and CAR (B2) T cells individually inhibited tumor growth in xenografts (FIG. 6B). Bi-hYP7−B2 CAR T cells failed to regress tumor burden and treatment with the bispecific CAR was less effective than mono-specific CAR-T cells, whereas the combination group hYP7 CAR+B2 CAR showed a significant synergistic anti-tumor effect in xenografts (FIG. 6B). Mice receiving CAR (B2) T, hYP7 CAR+B2 CAR T, or Bi-hYP7−B2 CAR T cells had a much higher absolute CD3+CAR+ T cell counts in blood compared with those receiving CAR (CD19) T or CAR (hYP7) T cells on week 2 after infusion (FIG. 6C). In both CD4+ and CD8+ T subpopulations, CAR (hYP7) T showed a higher proportion of memory stem cell-like (Tscm) T cells in mice than other CAR T cells, whereas B2-related CAR T cells had higher proportion of effector memory (Tem) T cells than CAR (hYP7) T cells. In vivo, CAR (hYP7) T cells expressed lower levels of PD-1 and LAG-3 than B2-related CAR T cells on week 2 after infusion (FIG. 6E).

### Example 10: Antitumor Activity of CAR (B2) T Cells in the Orthotopic MDA-MB-231 Mouse Model

To evaluate anti-tumor efficacy of CAR (B2) T in TNBC, an orthotopic xenograft mouse model was established via implanting MDA-MB-231 GFP-Luc tumor cells into mouse mammary fat pad. Seventeen days after tumor inoculation, mice were intravenously (i.v.) infused with either 5 million CAR (B2) T cells or antigen-mismatched CAR (CD19) T cells when the tumor median bioluminescence intensity was 1.77×10⁹ (FIG. 7A). Since the bioluminescence signal saturation of the IVIS imager occurred after 4 weeks of CAR (CD19) T infusion, both bioluminescence intensity and tumor volume were used to track the antitumor efficacy of CAR (B2) CAR T cells. Mice were followed up to 8 weeks post CAR T cell infusion except one CAR (CD19) mouse (#1) and two CAR (B2) mice (#2 and #3) were euthanized after week 3 for other analyses. Mice treated with CAR (B2) T cells showed significantly decreased tumor growth compared with CAR (CD19) T cell (FIGS. 7B and 7C), without a marked loss of body weight (FIG. 7D). After 5 weeks of CAR T infusions, tumors metastasized in the CAR (CD19) infusion-treated mice, but not in the CAR (B2) treated mice (FIG. 7B). In addition, no tumor metastases were found in the liver or lungs of mice that were treated with 5 million CAR (B2) T cells (FIG. 7E), indicating powerful antitumor efficacy of CAR (B2) T cells for treating metastatic lesions. Furthermore, it was found that CAR (B2) T cells had a comparable persistence in the spleen of two mice after 3 weeks of infusion (FIG. 7F). These spleen-isolated CAR (B2) T cells still have significant ex vivo cytotoxicity targeting MDA-MB-231 cells compared to MDA-MB-231 PD-L1 KO cells (FIG. 7G), which suggested that these in vivo persistent CAR (B2) T cells remained robust. By the end of week 8, mice were euthanized, and tumors were isolated from 6 mice to analyze antigen expression after CAR-T cell treatment in vivo. PD-L1 expression was normalized by tumor-specific GFP expression and it was found that there was no significant difference in PD-L1 expression between the CAR (CD19) T cell group and CAR (B2) T cell group (FIG. 7H).

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims. We therefore claim all that comes within the scope and spirit of these claims.

1. A polypeptide that specifically binds programmed death-ligand 1 (PD-L1), wherein the polypeptide comprises the complementarity determining region 1 (CDR1) and CDR3 sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12.

2. The polypeptide of claim 1, comprising:
the CDR1 and CDR3 sequences of SEQ ID NO: 1, wherein the CDR1 and CDR3 sequences respectively comprise residues 26-33 and 86-102, residues 26-33 and 84-102, or residues 22-35 and 86-102 of SEQ ID NO: 1;
the CDR1 and CDR3 sequences of SEQ ID NO: 2, wherein the CDR1 and CDR3 sequences respectively comprise residues 26-33 and 86-102, residues 26-33 and 84-102, or residues 22-35 and 86-102 of SEQ ID NO: 2;
the CDR1 and CDR3 sequences of SEQ ID NO: 3, wherein the CDR1 and CDR3 sequences respectively comprise residues 26-33 and 86-102, residues 26-33 and 84-102, or residues 22-35 and 86-102 of SEQ ID NO: 3;
the CDR1 and CDR3 sequences of SEQ ID NO: 4, wherein the CDR1 and CDR3 sequences respectively comprise residues 26-33 and 86-102, residues 26-33 and 84-102, or residues 22-35 and 86-102 of SEQ ID NO: 4;
the CDR1 and CDR3 sequences of SEQ ID NO: 5, wherein the CDR1 and CDR3 sequences respectively comprise residues 26-33 and 86-102, residues 26-33 and 84-102, or residues 22-35 and 86-102 of SEQ ID NO: 5;
the CDR1 and CDR3 sequences of SEQ ID NO: 6, wherein the CDR1 and CDR3 sequences respectively comprise residues 26-33 and 86-102, residues 26-33 and 84-102, or residues 22-35 and 86-102 of SEQ ID NO: 6;
the CDR1 and CDR3 sequences of SEQ ID NO: 7, wherein the CDR1 and CDR3 sequences respectively comprise residues 26-33 and 86-102, residues 26-33 and 84-102, or residues 22-35 and 86-102 of SEQ ID NO: 7;
the CDR1 and CDR3 sequences of SEQ ID NO: 8, wherein the CDR1 and CDR3 sequences respectively comprise residues 26-33 and 86-105, residues 26-33 and 84-105, or residues 22-35 and 86-105 of SEQ ID NO: 8;
the CDR1 and CDR3 sequences of SEQ ID NO: 9, wherein the CDR1 and CDR3 sequences respectively comprise residues 26-33 and 86-102, residues 26-33 and 84-102, or residues 22-35 and 86-102 of SEQ ID NO: 9;
the CDR1 and CDR3 sequences of SEQ ID NO: 10, wherein the CDR1 and CDR3 sequences respectively comprise residues 26-33 and 86-102, residues 26-33 and 84-102, or residues 22-35 and 86-102 of SEQ ID NO: 10;
the CDR1 and CDR3 sequences of SEQ ID NO: 11, wherein the CDR1 and CDR3 sequences respectively comprise residues 26-33 and 86-102, residues 26-33 and 84-102, or residues 22-35 and 86-102 of SEQ ID NO: 11; or
the CDR1 and CDR3 sequences of SEQ ID NO: 12, wherein the CDR1 and CDR3 sequences respectively comprise residues 26-33 and 86-102, residues 26-33 and 84-102, or residues 22-35 and 86-102 of SEQ ID NO: 12.

3. The polypeptide of claim 1, further comprising:
a hypervariable region 2 (HV2), wherein the HV2 sequence comprises SEQ ID NO: 15; and/or
a hypervariable region 4 (HV4), wherein the HV4 sequence comprises SEQ ID NO: 16.

4. A polypeptide that specifically binds programmed death-ligand 1 (PD-L1), wherein the polypeptide comprises a complementarity determining region 1 (CDR1), a hypervariable region 2 (HV2) and a CDR3, wherein the CDR1, HV2 and CDR3 sequences respectively comprise:
SEQ ID NO: 14, SEQ ID NO: 15 and residues 86-102 of SEQ ID NO: 1;
SEQ ID NO: 14, SEQ ID NO: 15 and residues 86-102 of SEQ ID NO: 2;
SEQ ID NO: 14, SEQ ID NO: 15 and residues 86-102 of SEQ ID NO: 3;
SEQ ID NO: 14, SEQ ID NO: 15 and residues 86-102 of SEQ ID NO: 4;
SEQ ID NO: 14, SEQ ID NO: 15 and residues 86-102 of SEQ ID NO: 5;
SEQ ID NO: 14, SEQ ID NO: 15 and residues 86-102 of SEQ ID NO: 6;
SEQ ID NO: 14, SEQ ID NO: 15 and residues 86-102 of SEQ ID NO: 7;
SEQ ID NO: 14, SEQ ID NO: 15 and residues 86-105 of SEQ ID NO: 8;
SEQ ID NO: 14, SEQ ID NO: 15 and residues 86-102 of SEQ ID NO: 9;
SEQ ID NO: 14, SEQ ID NO: 15 and residues 86-102 of SEQ ID NO: 10;
SEQ ID NO: 14, SEQ ID NO: 15 and residues 86-102 of SEQ ID NO: 11; or
SEQ ID NO: 14, SEQ ID NO: 15 and residues 86-102 of SEQ ID NO: 12.

5. (canceled)

6. The polypeptide of claim 1, wherein;
the amino acid sequence of the polypeptide is at least 90% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12; or
the amino acid sequence of the polypeptide comprises or consists of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12.

7. (canceled)

8. The polypeptide of claim 1, wherein the polypeptide is a single-domain antibody.

9. The polypeptide of claim 8, wherein:
The single-domain antibody is a shark variable new antigen receptor (V_{NAR}) antibody;
the single-domain monoclonal antibody is a humanized antibody; or
the single-domain monoclonal antibody is a chimeric antibody.

10-11. (canceled)

12. A fusion protein comprising the polypeptide of claim 1 and a heterologous protein.

13. The fusion protein of claim 12, wherein the heterologous protein comprises an Fc protein.

14. (canceled)

15. A chimeric antigen receptor (CAR) comprising the polypeptide of claim 1.

16. An isolated cell expressing the CAR of claim 15.

17. The isolated cell of claim 16, further expressing a CAR that specifically binds glypican-3 (GPC3).

18. The isolated cell of claim 16, wherein the cell is a T cell, a natural killer (NK) cell, a macrophage or an induced pluripotent stem cell (iPSC).

19. An immunoconjugate comprising the polypeptide of claim 1 and an effector molecule.

20. The immunoconjugate of claim 19, wherein the effector molecule is a toxin, a detectable label or a photon absorber.

21. An antibody-drug conjugate (ADC) comprising a drug conjugated to the polypeptide of claim 1.

22. A multi-specific antibody comprising the polypeptide of claim 1 and at least one additional monoclonal antibody or antigen-binding fragment thereof.

23. The multi-specific antibody of claim 22, which is a bispecific antibody.

24. The multi-specific antibody of claim 22, wherein the at least one additional monoclonal antibody or antigen-binding fragment specifically binds GPC3.

25. An antibody-nanoparticle conjugate, comprising a nanoparticle conjugated to the polypeptide of claim 1.

26. (canceled)

27. An isolated nucleic acid molecule encoding the polypeptide of claim 1.

28. The isolated nucleic acid molecule of claim 27, wherein the nucleic acid molecule encoding the polypeptide comprises or consists of any one of SEQ ID NOs: 17-28, or a degenerate variant thereof.

29. The isolated nucleic acid molecule of claim 27, operably linked to a promoter.

30. A vector comprising the nucleic acid molecule of claim 29.

31. An isolated host cell comprising the vector of claim 30.

32. A composition comprising a pharmaceutically acceptable carrier and the polypeptide of claim 1.

33. A method of detecting expression of PD-L1 in a sample, comprising:
contacting the sample with the polypeptide of claim 1;
detecting binding of the polypeptide to the sample, thereby detecting PD-L1 in the sample.

34-37. (canceled)

38. A method of treating a PD-L1-positive cancer in a subject, or a method of inhibiting tumor growth or metastasis of a PD-L1-positive cancer in a subject, comprising administering to the subject a therapeutically effective amount of the polypeptide of claim 1.

39. (canceled)

40. The method of claim 38, wherein the PD-L1-positive cancer is a solid tumor.

41. The method of claim 40, wherein the solid tumor is a liver cancer, a breast cancer, pancreatic cancer, melanoma, non-small cell lung cancer (NSCLC), renal cell carcinoma, a bladder cancer, head and neck squamous cell carcinoma (HNSCC), a gastric cancer, urothelial carcinoma, or Merkel cell carcinoma.

42-43. (canceled)
